(19) United States
(12) Patent Application Publication
(10) Pub. No.: US 2025/0221943 A1
(43) Pub. Date: Jul. 10, 2025

(54) POLYDOPAMINE CO-POLYMER NANOPARTICLES

(71) Applicant: CAMBRIDGE ENTERPRISE LIMITED, Cambridgeshire (GB)

(72) Inventors: Ljiljana Fruk, Cambridge (GB); Andrea Bistrovic Popov, Cambridge (GB)

(21) Appl. No.: 18/853,544

(22) PCT Filed: Apr. 4, 2023
(86) PCT No.: PCT/EP23/58902

§ 371 Date: Oct. 2, 2024

(30) Foreign Application Priority Data
Apr. 4, 2022 (GB) 2204935.7

(51) Int. Cl. A61K 9/51; A61K 47/64; A61P 35/00; B82Y 5/00

(52) U.S. Cl. CPC A61K 9/5146; A61K 9/5192; A61K 47/645; A61P 35/00; B82Y 5/00

(57) ABSTRACT

The present invention relates to polydopamine co-polymer nanoparticles as defined in the application. The present invention also relates to processes for the preparation of these polydopamine co-polymer nanoparticles, to pharmaceutical compositions comprising them, and to their use in therapy.

### FIELD OF THE INVENTION

The present invention relates to polydopamine co-polymer nanoparticles. The invention also relates to pharmaceutical compositions comprising the polydopamine co-polymer nanoparticles, processes for preparing the polydopamine co-polymer nanoparticles, and to particular applications of these nanoparticles.

### BACKGROUND OF THE INVENTION

Certain solid tumour cancers (such as pancreatic cancer) are characterised by marked intra- and inter-tumoral heterogeneity and a dense tumour microenvironment. Due to poor therapeutic responsiveness and lack of improvement in patient survival in the last 40 years, there is need to gain better understanding of the highly complex tumour biology and develop new strategies to improve treatment efficacy.

Nanocarriers present one of the most promising avenues for drug delivery. Although several nanomaterials have been approved for clinical use, the translation from the lab through preclinical assessment to the clinic remains challenging.

Approved drug loaded polymeric nanoparticles are described by Mitchell et al, Nature Reviews, Drug Discovery, Vol. 20, February 2021.

Pancreatic ductal adenocarcinoma (PDAC), as an example, is the fifth most common cause of cancer death in the UK, with a 5-year survival rate below 8% and without significant improvement in outcome over the last 40 years.^{[1,2]} Due to non-specific symptoms, PDAC is diagnosed at an advanced stage and characterized by early metastasis, a dense, heterogenous microenvironment and complex tumour biology. Current treatment relies mainly on chemotherapy as only 10-20% of patients can undergo surgery. However, pancreatic cancer shows resistance to both chemotherapy and radiotherapy.^{[3,4]} The stromal tissue contributes to the low therapeutic response and accounts for up to 80% of the total tumour volume. The stroma contains cellular components such as pancreatic stellate cells (PSCs), cancer-associated fibroblasts (CAFs), tumour-associated macrophages (TAMs), epithelial cells and pericytes, as well as non-cellular components such as the extracellular matrix (ECM), enzymes, cytokines and growth factors.^{[5-7]} These stromal components contribute to the high density, stiffness and interstitial pressure, acting as a shielding physical barrier to therapeutic delivery.

Nanocarriers have been extensively explored for the purpose of overcoming biological barriers, improving the pharmacokinetic profile of poorly soluble drugs as well as the pharmacological parameters such as clearance rate and peak drug concentration.^{[8]} So far, over 50 nanoformulations, including liposomes, polymers and albumin nanoparticles, have already been approved for clinical use, mostly in cancer therapy.^{[9,10]} Among those, albumin bound paclitaxel (Abraxane)^{[11]} and liposomal irinotecan (Onivyde),^{[12]} are approved for pancreatic cancer treatment. The complex tumour biology of pancreatic cancer has resulted in slower implementation of nanoformulations for treatment, with a marked increase in clinical trials in the past seven years.^{[13,14]} Although nanomaterials have remarkable properties for drug delivery applications, the lack of clinical translation led to the re-evaluation of the nanomedicine field in recent years.^{[15-20]} Poor understanding of disease heterogeneity and an inability to fine-tune the system based on the tumour biology was outlined as one of the most significant challenges.^{[15]} Additionally, aside from determining cytotoxicity and drug loading ability of the carriers during early-stage in vitro assessment, it is crucial to determine their immunocompatibilty, which is often overlooked until the latest stage of preclinical assessment.^{21-23]} Nanocarriers can be designed to be immunostimulatory, immunosuppressive or to evade the immune system altogether, and small changes in nanocarrier size, shape or charge can have a significant effect on their immunological profile and reduce their therapeutic efficiency.^{[24]}

In addition to altering the immune-response, physicochemical—(e.g. size, charge and hydrophobicity) and mechanical—(e.g. softness and rigidity) properties of nanocarriers are crucial for their transport and tissue penetration. Previous studies have shown that smaller particles (5100 nm) show better diffusion within solid tumours, but larger particles (100-150 nm) circulate longer in blood leading to increased accumulation in the target tissue.^{[25-27]} Therefore, it is advantageous to tailor the size of the nanocarriers to fit the application. For most polymer nanocarriers, size control is achieved by changing the chemical composition or surface functionalization, such as polyethylene glycol (PEG) chain length, which results in inconsistent observations of the size impact.^{[28]}

Besides enabling control over charge and size, surface functionalisation is often used to introduce a stealth layer, in order to minimise interactions with the immune system and blood components such as serum proteins and macrophages, which cause rapid clearance. The most commonly used stealth layer is polyethylene glycol (PEG) utilised in several clinically used nanoformulations.^{[29]} Although efficient in evading macrophages and other blood components, PEG can hinder cell internalisation and several studies indicated the presence of anti-PEG antibodies.^{[30-32]} Therefore alternative surface coatings need to be explored.

Pluronic F127 has been used as a templating agent to form porous polydopamine nanoparticles [40,41]. However, the inventors have surprisingly found that the covalent attachment of a polyethylene oxide-polypropylene oxide copolymer, such as Pluronic F127, to the polydopamine copolymer nanoparticles described herein enables nanoparticles of controllable particle size to be formed by simply changing the solvent ratio (ethanol to water) in the polymerisation reaction mixture. The nanoparticles formed have excellent colloidal stability, immunocompatibility, cell uptake, and have a surface that is hydrophilic and can be tuned to have a net neutral charge, which can facilitate penetration through the tumour extracellular matrix [35,36]. As demonstrated in the example section, the nanoparticles can also be functionalised by loading drugs, imaging agents etc.

Transfection in plant cells may allow using plant cells as bio factories for production of recombinant proteins (Qiang Chen, Huafang Lai, “Gene Delivery into Plant Cells for Recombinant Protein Production”, BioMed Research International, vol. 2015, Article ID 932161, 10 pages, 2015.). The use of carbon nanotube-based nanoparticles for transfection in plants has also been described by Demirer et al (Carbon nanotube-mediated DNA delivery without transgene integration in intact plants. Nat Protoc 14, 2954-2971 (2019)).

Ferulic acid has been demonstrated to bind to cellulose walls (Sieminska-Kuczer et al, Food Chemistry 373 (2022)). Further, the use of ferulic acid for drug delivery system has been described by Zhen et al (Adv. Funct. Mater. 2019, 29) and Romeo et al (Adv. Funct. Mater. 2019, 29).

RGD peptides have been described by Kang et al (Polymers 2020, 12(9), 1906) and then 1 for breast cancer (Diaz Bessone, M. I. et al. iRGD-guided tamoxifen polymersomes inhibit estrogen receptor transcriptional activity and decrease the number of breast cancer cells with self-renewing capacity. J. Nanobiotechnology 17, 120 (2019).), lung cancer (Zhang, Q. et al. A Novel Strategy to Improve the Therapeutic Efficacy of Gemcitabine for Non-Small Cell Lung Cancer by the Tumor-Penetrating Peptide iRGD. PLoS One 10, e0129865 (2015)), pancreatic cancer (Lo, J. H. et al. iRGD-guided Tumor-penetrating Nanocomplexes for Therapeutic siRNA Delivery to Pancreatic Cancer. Mol. Cancer Ther. 17, 2377-2388 (2018).) and glioblastoma (Gregory, J. V et al. Systemic brain tumor delivery of synthetic protein nanoparticles for glioblastoma therapy. Nat. Commun. 11, 5687 (2020)).

### SUMMARY OF THE INVENTION

In one aspect, the present invention provides polydopamine co-polymer nanoparticles as defined herein.

In another aspect, the present invention provides a pharmaceutical composition as defined herein which comprises polydopamine co-polymer nanoparticles as defined herein, and one or more pharmaceutically acceptable excipients, wherein the polydopamine co-polymer nanoparticles are loaded with a functional moiety as defined herein (for example a pharmacologically active agent).

In another aspect, the present invention provides a process for preparing polydopamine co-polymer nanoparticles as defined herein, the process comprising polymerising catecholamine (e.g. dopamine) or DOPAC monomer with a monomer of a catecholamine (e.g. dopamine) or DOPAC that is covalently bound to a co-polymer of poly(ethylene oxide) and poly(propylene oxide) to form polydopamine co-polymer nanoparticles comprising polydopamine having a co-polymer of poly(ethylene oxide) and poly(propylene oxide) covalently bound thereto.

In another aspect, the present invention provides polydopamine co-polymer nanoparticles obtainable by, obtained by or directly obtained by a process as defined herein.

In another aspect, the present invention provides polydopamine co-polymer nanoparticles as defined herein, or a pharmaceutical composition as defined herein, wherein the polydopamine co-polymer nanoparticles are loaded with a pharmacologically active agent.

In another aspect, the present invention provides polydopamine co-polymer nanoparticles as defined herein, or a pharmaceutical composition as defined herein, for use in therapy.

In another aspect, the present invention provides polydopamine co-polymer nanoparticles as defined herein, or a pharmaceutical composition as defined herein, wherein the polydopamine co-polymer nanoparticles are loaded with one or more of a pharmacologically active agent, an imaging agent, or a targeting moiety, for use in therapy.

Thus, the polydopamine co-polymer nanoparticles of the present invention are particularly suitable for use as a nanocarrier in drug delivery.

In another aspect, the present invention provides polydopamine co-polymer nanoparticles as defined herein, or a pharmaceutical composition as defined herein, wherein the polydopamine co-polymer nanoparticles are loaded with an anticancer agent, for use in the treatment of cancer.

In another aspect, the present invention provides polydopamine co-polymer nanoparticles as defined herein, or a pharmaceutical composition as defined herein, wherein the polydopamine co-polymer nanoparticles are loaded with an imaging agent, for use in medical imaging or photothermal therapy.

In another aspect, the present invention provides polydopamine co-polymer nanoparticles as defined herein, or a pharmaceutical composition as defined herein, wherein the polydopamine co-polymer nanoparticles are loaded with an imaging agent, for use as:
(i) a photoacoustic imaging agent;
(ii) a photothermal therapeutic agent;
(iii) a magnetic resonance imaging (MRI) agent (i,e, an MRI contrast agent); or
(iv) a positron emission tomography (PET) imaging agent.

In another aspect, the present invention provides the use of polydopamine co-polymer nanoparticles as defined herein, or a pharmaceutical composition as defined herein, wherein the polydopamine co-polymer nanoparticles are loaded with one or more of a pharmacologically active agent, an imaging agent, or a targeting moiety, in the manufacture of a medicament for use in therapy.

In another aspect, the present invention provides the use of polydopamine co-polymer nanoparticles as defined herein, or a pharmaceutical composition as defined herein, wherein the polydopamine co-polymer nanoparticles are loaded with an anticancer agent, in the manufacture of a medicament for use in the treatment of cancer.

In another aspect, the present invention provides the use of polydopamine co-polymer nanoparticles as defined herein, or a pharmaceutical composition as defined herein, wherein the polydopamine co-polymer nanoparticles are loaded with an imaging agent, in the manufacture of a medicament for use in medical imaging or photothermal therapy.

In another aspect, the present invention provides the use of polydopamine co-polymer nanoparticles as defined herein, or a pharmaceutical composition as defined herein, wherein the polydopamine co-polymer nanoparticles are loaded with an imaging agent, in the manufacture of a medicament for use as:
(i) a photoacoustic imaging agent;
(ii) a photothermal therapeutic agent;
(iii) a magnetic resonance imaging (MRI) agent (i,e, an MRI contrast agent); or
(iv) a positron emission tomography (PET) imaging agent.

In another aspect, the present invention provides a method of treating cancer in a patient in need of such treatment, said method comprising administering to said patient a therapeutically effective amount of polydopamine co-polymer nanoparticles as defined herein, or a pharmaceutical composition as defined herein, wherein the polydopamine co-polymer nanoparticles are loaded with an anticancer agent.

In another aspect, the present invention provides a method of:
(i) medical imaging; or
(ii) photothermal therapy;
in a patient in need of such treatment, said method comprising administering to said patient a therapeutically effective amount of polydopamine co-polymer nanoparticles as defined herein, or a pharmaceutical composition as defined herein, wherein the polydopamine co-polymer nanoparticles are loaded with an imaging agent.

In another aspect, the present invention provides a method of:
(i) photoacoustic imaging;
(ii) photothermal therapy;
(iii) magnetic resonance imaging; or
(iv) positron emission tomography (PET) scanning;
in a patient in need of such treatment, said method comprising administering to said patient a therapeutically effective amount of polydopamine co-polymer nanoparticles as defined herein, or a pharmaceutical composition as defined herein, wherein the polydopamine co-polymer nanoparticles are loaded with an imaging agent.

The polydopamine co-polymer nanoparticles described herein may be used in the delivery of nucleic acids to cells, for example when functionalised with a nucleic acid. The nucleic acid may be DNA (e.g. plasmid DNA) or RNA (including sRNA).

In another aspect, the present invention provides polydopamine co-polymer nanoparticles as defined herein, or a pharmaceutical composition as defined herein, wherein the polydopamine co-polymer nanoparticles are functionalised with a nucleic acid.

In another aspect, the present invention provides polydopamine co-polymer nanoparticles as defined herein, or a pharmaceutical composition as defined herein, wherein the polydopamine co-polymer nanoparticles are functionalised with a nucleic acid, for use in the transfection of cells in vitro or in vivo.

In another aspect, the present invention provides polydopamine co-polymer nanoparticles as defined herein, or a pharmaceutical composition as defined herein, wherein the polydopamine co-polymer nanoparticles are functionalised with a nucleic acid, for use in the transfection of cells in plants.

In another aspect, the present invention provides polydopamine co-polymer nanoparticles as defined herein, or a pharmaceutical composition as defined herein, wherein the polydopamine co-polymer nanoparticles are functionalised with a nucleic acid, for use in the transfection of human or animal cells.

In another aspect, the present invention provides polydopamine co-polymer nanoparticles as defined herein, or a pharmaceutical composition as defined herein, wherein the polydopamine co-polymer nanoparticles are functionalised with a nucleic acid, for use in the transfection of cells in therapy.

In another aspect, the present invention provides polydopamine co-polymer nanoparticles as defined herein, or a pharmaceutical composition as defined herein, wherein the polydopamine co-polymer nanoparticles are functionalised with a nucleic acid, for use in the delivery of nucleic acids in therapy.

In another aspect, the present invention provides the use of polydopamine co-polymer nanoparticles as defined herein, or a pharmaceutical composition as defined herein wherein the polydopamine co-polymer nanoparticles are functionalised with a nucleic acid, in the manufacture of a medicament for use in the delivery of nucleic acids in therapy.

In another aspect, the present invention provides a method for the transfection of cells in vitro or in vivo, said method comprising contacting a cell with polydopamine co-polymer nanoparticles as defined herein, wherein the polydopamine co-polymer nanoparticles are functionalised with a nucleic acid.

In another aspect, the present invention provides a method for the transfection of cells in vivo, said method comprising contacting a cell with polydopamine co-polymer nanoparticles as defined herein, wherein the polydopamine co-polymer nanoparticles are functionalised with a nucleic acid.

In another aspect, the present invention provides a method for the transfection of plant cells, said method comprising contacting a cell with polydopamine co-polymer nanoparticles as defined herein, wherein the polydopamine co-polymer nanoparticles are functionalised with a nucleic acid.

In another aspect, the present invention provides a method for the delivery of nucleic acids to a cell, said method comprising contacting a cell with polydopamine co-polymer nanoparticles as defined herein, wherein the polydopamine co-polymer nanoparticles are functionalised with a nucleic acid. Suitably, the cell is an human cell, animal cell or a plant cell.

The polydopamine co-polymer nanoparticles may be further functionalised with a poly(amino acid) which is positively charged at pH 7 and the nucleic acid. The poly(amino acid) may be one or more of poly-L-histidine, poly-L-arginine or poly-L-lysine. More suitably, the poly(amino acid) is selected from poly-L-histidine or poly-L-arginine, or a combination thereof.

The polydopamine co-polymer nanoparticles may be further functionalised with ferulic acid and the nucleic acid.

In another aspect, the present invention provides polydopamine co-polymer nanoparticles as defined herein, or a pharmaceutical composition as defined herein, wherein the polydopamine co-polymer nanoparticles are functionalised with a poly(amino acid) which is positively charged at pH 7, for use in the transfection of cells in vitro or in vivo. The poly(amino acid) may be one or more of poly-L-histidine, poly-L-arginine or poly-L-lysine. The poly(amino acid) may be one or more of poly-L-histidine or poly-L-arginine.

In another aspect, the present invention provides polydopamine co-polymer nanoparticles as defined herein, or a pharmaceutical composition as defined herein, wherein the polydopamine co-polymer nanoparticles are functionalised with a poly(amino acid) which is positively charged at pH 7, for use in the transfection of cells in plants. The poly(amino acid) may be one or more of poly-L-histidine, poly-L-arginine or poly-L-lysine. The poly(amino acid) may be one or more of poly-L-histidine or poly-L-arginine.

In another aspect, the present invention provides polydopamine co-polymer nanoparticles as defined herein, or a pharmaceutical composition as defined herein, wherein the polydopamine co-polymer nanoparticles are functionalised with a poly(amino acid) which is positively charged at pH 7, for use in the transfection of human or animal cells. The poly(amino acid) may be one or more of poly-L-histidine, poly-L-arginine or poly-L-lysine. The poly(amino acid) may be one or more of poly-L-histidine or poly-L-arginine.

In another aspect, the present invention provides polydopamine co-polymer nanoparticles as defined herein, or a pharmaceutical composition as defined herein, wherein the polydopamine co-polymer nanoparticles are functionalised with a poly(amino acid) which is positively charged at pH 7, for use in the transfection of cells in therapy. The poly(amino acid) may be one or more of poly-L-histidine, poly-L-arginine or poly-L-lysine. The poly(amino acid) may be one or more of poly-L-histidine or poly-L-arginine.

In another aspect, the present invention provides polydopamine co-polymer nanoparticles as defined herein, or a pharmaceutical composition as defined herein, wherein the polydopamine co-polymer nanoparticles are functionalised with a poly(amino acid) which is positively charged at pH 7, for use in the delivery of nucleic acids in therapy.

In another aspect, the present invention provides the use of polydopamine co-polymer nanoparticles as defined herein, or a pharmaceutical composition as defined herein, wherein the polydopamine co-polymer nanoparticles are functionalised with a poly(amino acid) which is positively charged at pH 7, in the manufacture of a medicament for use in the delivery of nucleic acids in therapy. The poly(amino acid) may be one or more of poly-L-histidine, poly-L-arginine or poly-L-lysine. The poly(amino acid) may be one or more of poly-L-histidine or poly-L-arginine.

In another aspect, the present invention provides the use of polydopamine co-polymer nanoparticles as defined herein, or a pharmaceutical composition as defined herein, wherein the polydopamine co-polymer nanoparticles are functionalised with a poly(amino acid) which is positively charged at pH 7, for use in the immobilisation of a nucleic acid (e.g DNA such as plasmid DNA, pDNA). The poly(amino acid) may be one or more of poly-L-histidine, poly-L-arginine or poly-L-lysine. The poly(amino acid) may be one or more of poly-L-histidine or poly-L-arginine.

In another aspect, the present invention provides a method for the transfection of cells in vitro or in vivo, said method comprising contacting a cell with polydopamine co-polymer nanoparticles as defined herein, wherein the polydopamine co-polymer nanoparticles are functionalised with a poly(amino acid) which is positively charged at pH 7 and a nucleic acid.

In another aspect, the present invention provides a method for the transfection of cells in vivo, said method comprising contacting a cell with polydopamine co-polymer nanoparticles as defined herein, wherein the polydopamine co-polymer nanoparticles are functionalised with a poly(amino acid) which is positively charged at pH 7 and a nucleic acid.

In another aspect, the present invention provides a method for the transfection of plant cells, said method comprising contacting a cell with polydopamine co-polymer nanoparticles as defined herein, wherein the polydopamine co-polymer nanoparticles are functionalised with a poly(amino acid) which is positively charged at pH 7 and a nucleic acid.

In another aspect, the present invention provides a method for the delivery of nucleic acids to a cell, said method comprising contacting a cell with polydopamine co-polymer nanoparticles as defined herein, wherein the polydopamine co-polymer nanoparticles are functionalised with a poly(amino acid) which is positively charged at pH 7 and a nucleic acid. Suitably, the cell is a human cell, animal cell or a plant cell.

In another aspect, the present invention provides a method for the immobilisation of a nucleic acid (e.g DNA), said method comprising contacting a nucleic acid with polydopamine co-polymer nanoparticles as defined herein, wherein the polydopamine co-polymer nanoparticles are functionalised with a poly(amino acid) which is positively charged at pH 7.

### BRIEF DESCRIPTION OF THE DRAWINGS

Embodiments of the invention are further described hereinafter with reference to the accompanying drawings, in which:

FIG. 1. Schematic illustration for the synthesis and evaluation of biocompatible F127@PDA NPs for drug-delivery application.

FIG. 2. Characterisation of F127@PDA. SEM images of F127@PDA NPs prepared in a 20:1 molar ratio of DA:F127DA with 10, 20, 35 and 50% ethanol in water (A). Linear correlation between the molar ratio of DA:F127DA and the solvent ratio of EtOH:H₂O with the hydrodynamic size of F127@PDA NPs (B). Hydrodynamic size evolution over time during the formation of F127@PDA_40 and F127@PDA_100 prepared in a 20:1 molar ratio of DA:F127DA with 10% and 35% EtOH in the reaction mixture, respectively (C).

FIG. 3. In vitro toxicity of F127@PDA NPs over 72 h. Brightfield images of different PDAC control cells and cells treated with 100 μg/mL F127@PDA_40 for 72 h (A). In vitro cytotoxicity of F127@PDA_40, F127@PDA_60 and F127@PDA_100 in AsPC-1, BxPC-3, MIA PaCa-2 and PANC-1 cell lines after 72 h incubation determined by live cell analysis. Data are expressed as mean±SD obtained from three separate measurements.

FIG. 4. Intercellular localization of F127@PDA NPs. Fluorescent images of PDAC cells (AsPC-1, BxPC-3, MIA PaCa-2 and PANC-1) after 24 h incubation with 50 μg/mL F127@PDA@FI_40 NPs acquired with confocal microscopy. Cells were incubated with Cell Mask (deep red) and Hoechst 33342 (blue) to stain cell membrane and nuclei, respectively. Green channel captured the fluorescence of NPs.

FIG. 5. Quantification of F127@PDA uptake using flow cytometry. Cells containing fluorescent NPs is assigned by cells of higher fluorescence intensity than the threshold intensity (dotted grey vertical line) of the untreated samples (A). Percentage of cells containing F127@PDA@FI NPs in different cell types incubated with different concentrations of NPs obtained from the forward scattering (FSC) vs FITC graph after flow cytometry analysis (B).

FIG. 6. Cytokine profiling of THP-1 and THP-1 M(0) cells after 24 h incubation with 10 μg/mL F127@PDA_40, F127@PDA_60 and F127@PDA_100 NPs. LPS is used as (+) control. Data is expressed as mean±SD. Two-way ANOVA was used for statistical analysis. Significance levels are defined as the following: ns for p>0.05, * for p≤0.05, ** for p≤0.01, *** for p<0.001, and **** for p<0.0001 compared to the Control (−).

FIG. 7. Cytotoxicity of SN38 and SN38@F127@PDA and F127@PDA NPs determined by live-cell imaging after 72 h treatment. Data is expressed as mean±SD from three experiments. Two-way ANOVA was used to compare SN38 and SN38@F127@PDA. Significance levels are defined as the following: ns for p>0.05, * for p≤0.05, ** for p≤0.01, *** for p<0.001, and **** for p<0.0001.

FIG. 8. ¹H NMR spectra of F127, F127COOH and F127DA in CDCl₃.

FIG. 9. FT-IR spectra obtained for F127, F127COOH and F127DA with KBr pellet.

FIG. 10. ¹H NMR spectra of F127 in CDCl₃.

FIG. 11. ¹H NMR spectra of F127COOH in CDCl₃.

FIG. 12. ¹H NMR spectra of F127DA in CDCl₃.

FIG. 13. SEM images of F127@PDA nanoparticles prepared in a 10:1 (A), 20:1 (B), 50:1 (C), 100:1 (D), 0:1 (D) and 1:0 (E) molar ratio of DA:F127DA with 35% ethanol in the reaction mixture.

FIG. 14. UV-Vis spectra of the reaction mixture during the formation of F127@PDA_40 (A) and F127@PDA_100 (B). Time-resolved dynamic light scattering (DLS) monitoring the evolution of hydrodynamic diameter distributions of the reaction suspension at different reaction times for F127@PDA_40 (C) and F127@PDA_100 (D).

FIG. 15. UV-Vis spectra of F127DA, F127@PDA_40, F127@PDA_60 and F127@PDA_100 in water at a concentration of 0.1 mg/mL.

FIG. 16. Colloidal stability of F127@PDA_40 (A), F127@PDA_60 (B) and F127@PDA_100 (C) measured using DLS (top) and UV-Vis spectroscopy (bottom).

FIG. 17. Spectroscopic characterization of F127@PDA@FI. UV-Vis spectra of F127@PDA@FI_40, F127@PDA@FI_60 and F127@PDA_100 (A). Fluorescence spectra of F127@PDA@FI_40, F127@PDA@FI_60 and F127@PDA_100 in water (λₑₓ=492 nm) (B). UV-Vis spectra of F127@PDA_40 and F127@PDA@FI_40 in water (C). Calibration curve for F127-TEG-NH₂ measured in water (D).

FIG. 18. Endocytic profiling within different PDAC cells: nuclei (blue stain), tubulin (green stain), Golgi (red stain) (A). Summary of IC₅₀ values obtained from Sanger drug screening data (https://www.cancerrxgene.org/)^{[100]} for SN-38, Paclitaxel and Gemcitabine (B).

FIG. 19. In vitro cytotoxicity effect of F127@PDA_40 (black and grey squares, left bars), F127@PDA_60 (grey central bars) and F127@PDA_100 (light grey right bars) on AsPC-1, BxPC-3, MIA PaCa-2 and PANC-1 after 72 h incubation determined by MTS assay. Data are expressed as the mean±SD.

FIG. 20. Orthogonal z-stack images of AsPC-1 (A), BxPC-3 (B), MIA PaCa-2 (C) and PANC-1 (D) after 24 h incubation with 50 μg/mL F127@PDA@FI_40 NPs acquired with confocal microscopy. Frontal view represents X-Y direction, top panel X-Z direction and right panel Y-Z direction. Cells were incubated with Cell Mask (deep red) and Hoechst 33342 (blue) to stain cell membrane and nuclei, respectively. Green dots represent the NPs.

FIG. 21. Effect of NP treatment on the side scatter of BxPC-3 cells (A). Histogram showing normalized mean fluorescence values of AsPC-1, BxPC-3, MIAPaCa-2, PANC-1 THP-1 (MO) and THP-1 cells treated with 50 μg/mL F127@PDA@FI_40, F127@PDA@FI_60 and F127@PDA@FI_100 for 24 h. MFI are represented as mean values and standard deviations of triplicate experiments (B).

FIG. 22. In vitro cytotoxicity effect of F127@PDA_40 (dark grey left bar), F127@PDA_60 (grey central bar) and F127@PDA_100 (light grey right bar) on THP-1 and THP-1 (MO) cells after 72 h incubation determined by live MTS assay (A). Data are expressed as the mean±SD. Microscopic images of monocyte-like THP-1 (B) and PMA differentiated macrophages THP1 (MO) (C).

FIG. 23. Drug loading of SN38. UV-Vis spectra of F127@PDA, SN38 and SN38@F127@PDA (A) and calibration curve of SN-38 (B) measured in methanol using a 1 mL quartz cuvette. Example of HPLC spectra showing both lactone and carboxylate form of SN38. (C) Calibration curve of SN38 obtained for lactone (D) and carboxylate form (E) using HPLC. Cumulative release of SN38 in PBS (1×, pH=7.4) during 72 h incubation at 37° C. (F).

FIG. 24. Growth curves of AsPC-1, BxPC-3, MIA PaCa-2 and PANC-1 obtained by live cell imaging during 72 h treatment with 1 nM (A) and 10 nM (B) SN38, SN38@F127@PDA and F127@PDA.

FIG. 25. Cytotoxicity of SN38 and SN38@F127@PDA and F127@PDA NPs determined by MTS assay imaging after 72 h treatment of AsPC-1, BxPC-3, MIA PaCa-2 and PANC-1.

FIG. 26. UV-Vis spectra Rhoadmine@F127@PDA (A), TAMRA-NLS@F127@PDA (B) and ATRA-MMP@F127@PDA (C) and the corresponding calibration curves for ligands 4 (D), 5 (E) and 6 (F) in Scheme 1.

FIG. 27. UV-Vis spectra and calibration curves for Gem in water (A) and PTX in methanol (B).

FIG. 28. UV-Vis spectra of F127PyDA monomer and F127Py@PDA NPs (A), F127@PyPDA NPs prepared using different ethanol amount (B) and their corresponding fluorescence spectra (C)

FIG. 29. SEM images of F127Py@PDA NPs prepared with 50% EtOH and molar ratios of n(DA):n(F127PyDA)=30:1(A), 75:1 (B) and 115:1 (C). F127@PyPDA NPs prepared using different ethanol amount (B) and their corresponding fluorescence spectra (C). SEM image of F127PyPDA NPs prepared with 25% EtOH and a molar ratio of n(DA):n(F127PyDA)=115:1 (D). Linear correlation of the hydrodynamic diameter of F127Py@PDA vs. the monomer ratio with 50% EtOH (E) and vs. EtOH amount with n(DA):n(F127PyDA)=115:1 (F)

FIG. 30. Schematic representation of the post-functionalization of Pluronic-polydopamine (F127@PDAF127PDA) NPs with poly-L-histidine and poly-L-arginine resulting in pHis-pArg-F127@PDAF127PDA NPs.

FIG. 31. Binding studies of pDNA to pHis-pArg-F127@PDAF127PDA NPs. Gel electrophoresis of pDNA@pHis-pArg-F127@PDAF127PDA_40 and pDNA@pHis-pArg-F127@PDAF127PDA_100 formulations ranging from WR-5 to WR-250.

FIG. 32. Transfection efficacy of pHis-pArg-F127@PDAF127PDA NPs in HEK-293 cells. Micrographs of HEK-293 cells in the green (EGFP) and bright field channel using a 20× objective with the IncuCyte treated with Lipofectamine, pHis-pArg-F127@PDAF127PDA_40 WR-5WR5 and pHis-pArg-F127@PDAF127PDA_100 WR-5WR5 after 24 h (A) and 48 h (B). Scale bar is 200 μm. Transfection efficacy of Lipofectamine, pHis-pArg-F127@PDAF127PDA_40 WR-5WR5 and pHis-pArg-F127@PDAF127PDA_100 WR-5WR5 over 48 h (C). Transfection efficacy of pHis-pArg-F127@PDAF127PDA_40 and pHis-pArg-F127@PDAF127PDA_100 using different NP to pDNA weight ratios (WR), (D).

FIG. 33. Time-dependent uptake of Rhodamine@F127@PDAF127PDA NPs (Rh@F127@PDAF127PDA) in BxPC-3, Capan-1, PANC-1 and hPSC cells.

FIG. 34. Efficacy of SN38@F127@PDAF127PDA (A) and Gem@F127@PDAF127PDA (B) NPs in pancreatic cancer cells (BxPC-3, Capan-1 and PANC-1) and human pancreatic stellate cells (hPSC). The cells were treated for 18 h with the formulations followed by a 96 h recovery. One-way ANOVA was used to compare the free drug with the F127@PDAF127PDA formulations. Significance levels are defined as the following: ns for p>0.05, * for p<0.05, ** for p≤0.01, *** for p<0.001, and **** for p<0.0001.

FIG. 35. Efficacy of SN38@F127PDA NPs in pancreatic cancer spheroids. PANC-1:hPSC spheroids with a cell ratio of 5:1 and 1:1 were used to study the drug efficacy. The spheroids were treated for 18 h with the formulations followed by a 96 h recovery. One-way ANOVA was used to compare SN38 and SN38@F127PDA formulations. Significance levels are defined as the following: ns for p>0.05, * for p≤0.05, ** for p≤0.01, *** for p<0.001, and **** for p<0.0001.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of polydopamine co-polymer nanoparticles loaded with an active agent that, when administered to a mammal for treating a disease, is sufficient to affect such treatment for the disease. The “therapeutically effective amount” will vary depending on the active agent, the disease and its severity and the age, weight, etc., of the mammal to be treated.

The phrase “polydopamine co-polymer nanoparticles of the invention” means those polydopamine co-polymer nanoparticles which are disclosed herein, both generically and specifically.

The term “polydopamine” is used to refer to polymers of catecholamine monomers (e.g. dopamine, epinephrine and L-DOPA) or DOPAC monomers. Suitably, a polydopamine is a polymer of a catecholamine monomer (e.g. norepinephrine, dopamine, epinephrine). Most suitably, a polydopamine is a polymer of dopamine monomer.

A catecholamine monomer is taken to refer to a catecholamine compound such as:

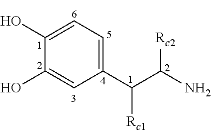

wherein R_{c1} is H or OH, and R_{c2} is H or C(═O)—OH. Particular examples include norepinephrine, dopamine and L-DOPA. Preferably, a catecholamine monomer is dopamine monomer.

A “dopamine monomer” is therefore taken to refer to the compound dopamine, which has the structure below:

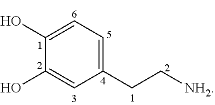 [SMILES: NCCc1ccc(O)c(O)c1]

A “DOPAC monomer” is taken to refer to the compound 3,4-dihydroxyphenylacetic acid (DOPAC), which has the structure below:

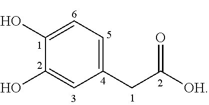 [SMILES: O=C(O)Cc1ccc(O)c(O)c1]

In the context of the present invention, a “nanoparticle” is taken to mean any particle with a size of 300 nm or less.

A “nanocarrier” is understood to be a nanomaterial (e.g. a nanoparticle) being used as a transport module for another substance, such as a drug or other functional moeity. Nanocarriers are useful in the drug delivery process because they can deliver drugs to site-specific targets, allowing drugs to be delivered in certain organs or cells but not in others. Site-specificity is a major therapeutic benefit since it prevents drugs from being delivered to the wrong places.

### Nanoparticles of the Invention

In one aspect, the present invention relates to polydopamine co-polymer nanoparticles comprising polydopamine having a co-polymer of poly(ethylene oxide) and poly(propylene oxide) covalently bound thereto.

Suitably, the polydopamine co-polymer nanoparticles have a particle size of less than or equal to 140 nm. Suitably, the polydopamine co-polymer nanoparticles have a particle size of from 30 to 140 nm. More suitably the polydopamine co-polymer nanoparticles have a particle size of from 40 to 100 nm. Most suitably the polydopamine co-polymer nanoparticles have a particle size of from 40 to 60 nm.

Suitably, the co-polymer of poly(ethylene oxide) and poly(propylene oxide) is a block co-polymer. More suitably, the co-polymer of poly(ethylene oxide) and poly(propylene oxide) is a tri-block co-polymer having a central block of poly(propylene oxide) flanked on each side by blocks of poly(ethylene oxide).

Suitably, the co-polymer has the formula:

-[poly(ethylene oxide)]-[poly(propylene oxide)]-[poly(ethylene oxide)]-

or

—X₁-[poly(ethylene oxide)]-[poly(propylene oxide)]-[poly(ethylene oxide)]-X₂—

wherein:
X₁ and X₂ are each independently:
   a linker group that connects the co-polymer to the polydopamine; or
   a detectable moiety (e.g. a fluorophore) that connects the co-polymer to the polydopamine.

More suitably, the co-polymer has the formula:

-[poly(ethylene oxide)]-[poly(propylene oxide)]-[poly(ethylene oxide)]-

or

—X₁-[poly(ethylene oxide)]-[poly(propylene oxide)]-[poly(ethylene oxide)]-X₂—
wherein X₁ and X₂ are each independently as defined herein.

Suitably, the co-polymer has the formula:

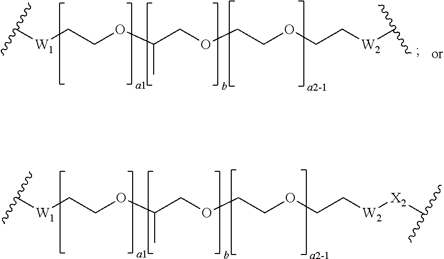

wherein:
W₁ and W₂ are selected from O or NH; or
W₁ and W₂ are selected from:

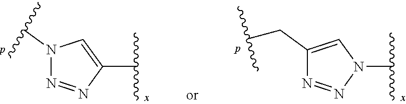 [SMILES: *c1cn(*)nn1 ; *Cc1cn(*)nn1]

wherein
p indicates the bond to the co-polymer;
x indicates the bond to the X₁ or X₂ group, if present, or the polydopamine;
a1 is an integer between 50 and 150;
a2 is an integer between 50 and 150;
b is an integer between 20 and 80; and
X₁ and X₂ are as defined herein.

In the repeating unit group labelled “a2-1”, this indicates that the number of repeating units is the value of a2 minus 1. Thus, when a2 is 101, then there would be 100 of the repeating units labelled “a2-1”.

Suitably, in certain embodiments, the co-polymer has the formula:

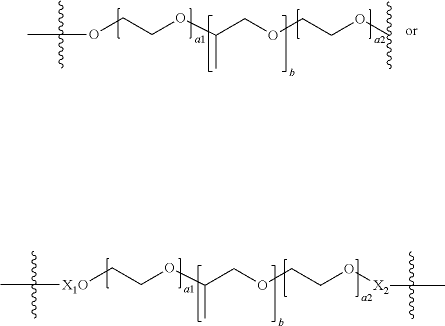

wherein
   a1 is an integer between 50 and 150;
   a2 is an integer between 50 and 150;
   b is an integer between 20 and 80; and
   X₁ and X₂ are as defined herein.

Suitably, a1 and a2 are the same.

Particular polydopamine co-polymer nanoparticles of the invention comprise a block co-polymer as defined herein, wherein unless otherwises stated, a1, a2, b, W₁, W₂, X₁ and X₂ have any of the meanings defined hereinbefore or in any of paragraphs (1) to (12) hereinafter:—
(1) a1 is an integer between 75 and 125;
   a2 is an integer between 75 and 125; and
   b is an integer between 25 and 75.
(2) a1 is an integer between 90 and 110;
   a2 is an integer between 90 and 110; and
   b is an integer between 45 and 65.
(3) a1 is 101;
   a2 is 101; and
   b is 56 (i.e. Pluronic F127).
(5) W₁ and W₂ are selected from O or NH, and
   X₁ and X₂ are each independently selected from:
   (i) a linker of the formula:

—C(═O)—[CH₂]ₙ—C(═O)—
wherein:
n is an integer from 1 to 10;
(ii) a linker of the formula:

—[CH₂]_{q}—(N(H))ᵣ—
wherein:
q is an integer from 0 to 10;
r is an integer selected from 0 or 1; or
(iii) a detectable moiety, e.g. a fluorophore.
(6) W₁ and W₂ are selected from O or NH; and
   X₁ and X₂ are each independently:
   (i) a linker of the formula:

—C(═O)—[CH₂]ₙ—C(═O)—
wherein:
n is an integer from 1 to 10; preferably from 1 to 5; or
(ii) a fluorophore.
(7) W₁ and W₂ are selected from O or NH; and
   X₁ and X₂ are each independently:
      (i) a linker of the formula:

—C(═O)—[CH₂]ₙ—C(═O)—
wherein n is an integer from 1 to 10; or
(ii) a fluorophore of the formula:

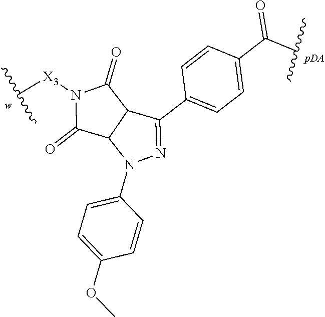

wherein:
   w indicates the bond to the W₁ or W₂ group;
   pDA indicates the bond to the polydopamine;
   X₃ is selected from:
   (i) —C(═O)—[CH₂]ₙ₁—. wherein n1 is an integer from 2 to 10;
   (ii) —[CH₂]ₙ₂—, wherein n2 is an integer from 2 to 10;
   (iii) —C(═O)—CH₂CH₂—[OCH₂CH₂]ₙ₃—, n3 is an integer from 2 to 10;
(iii) a fluorophore of the formula:

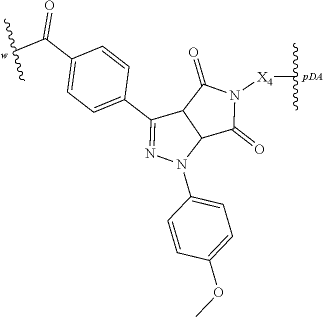

wherein:
   w indicates the bond to the W₁ or W₂ group;
   pDA indicates the bond to the polydopamine;
   X₄ is selected from:
   (i) —[CH₂]ₙ₄—C(═O). wherein n4 is an integer from 2 to 10;
   (ii) —[CH₂]ₙ₅—, wherein n5 is an integer from 2 to 10;
   (iii) —[CH₂CH₂O]ₙ₆—CH₂CH₂—C(═O)—, wherein n6 is an integer from 2 to 10.
(8) W₁ and W₂ are selected from O or NH; and
   X₁ and X₂ are each independently:
      (i) a linker of the formula:

—C(═O)—[C H₂]ₙ—C(═O)—
wherein n is an integer from 2 to 8; or
(ii) a fluorophore of the formula:

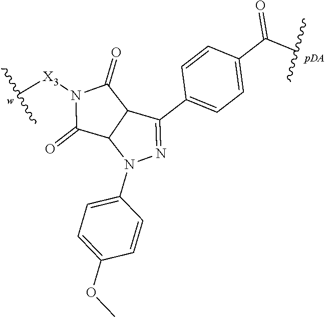

wherein:
   w indicates the bond to the W₁ or W₂ group;
   pDA indicates the bond to the polydopamine;
   X₃ is selected from:
   (i) —C(═O)—[CH₂]ₙ₁—. wherein n1 is an integer from 2 to 8;
   (ii) —[CH₂]ₙ₂—, wherein n2 is an integer from 2 to 8;
   (iii) —C(═O)—CH₂CH₂—[OCH₂CH₂]ₙ₃—, n3 is an integer from 2 to 8;
(iii) a fluorophore of the formula:

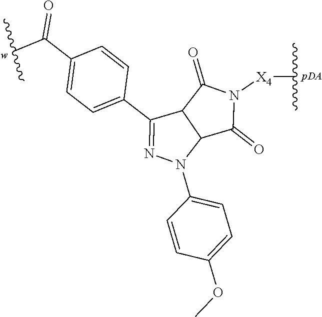

wherein:
   w indicates the bond to the W₁ or W₂ group;
   pDA indicates the bond to the polydopamine;
   X₄ is selected from:
   (i) —[CH₂]ₙ₄—C(═O). wherein n4 is an integer from 2 to 8;
   (ii) —[CH₂]ₙ₅—, wherein n5 is an integer from 2 to 8;
   (iii) —CH₂CH₂—[CH₂CH₂O]ₙ₆—C(═O)—, wherein n6 is an integer from 2 to 8.
(9) W₁ and W₂ are selected from O or NH; and
   X₁ and X₂ are each independently:
      (i) a linker of the formula:

—C(═O)—[CH₂]ₙ—C(═O)—
wherein n is an integer from 2 to 8; or
(ii) a fluorophore of the formula:

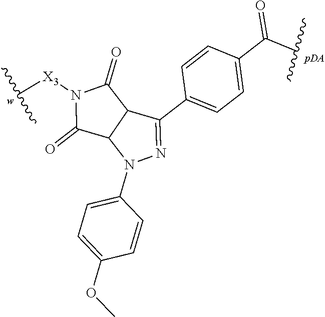

wherein:
   w indicates the bond to the W₁ or W₂ group;
   pDA indicates the bond to the polydopamine;
   X₃ is —C(═O)—[CH₂]ₙ₁—. wherein n1 is an integer from 2 to 8
(10) W₁ and W₂ are selected from O or NH; and
   X₁ and X₂ are each independently either:
      (i) a linker of the formula:

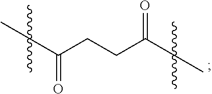 [SMILES: *C(=O)CCC(*)=O]

or
(ii) a fluorophore of the formula:

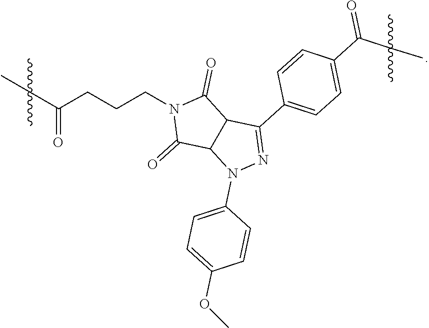 [SMILES: *C(=O)CCCN1C(=O)C2C(c3ccc(C(*)=O)cc3)=NN(c3ccc(OC)cc3)C2C1=O]

(11) W₁ and W₂ are each a group of the formula:

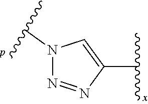 [SMILES: *c1cn(*)nn1]

wherein
p indicates the bond to the co-polymer;
x indicates the bond to the X₁ or X₂ group; and
X₁ and X₂ are each selected from:
(i) —CH₂—
(ii) —CH₂CH₂—[OCH₂CH₂]ₙ₇—, wherein n7 is an integer from 2 to 10;
(iii) —CH₂—C(═O)—
(iv) —CH₂CH₂—[OCH₂CH₂]ₙ₈—NH—C(═O)—, wherein n8 is an integer from 2 to 10;
(v) —CH₂CH₂—[OCH₂CH₂]ₙ₉—O—C(═O)—, wherein n9 is an integer from 2 to 10
(12) W₁ and W₂ are each a group of the formula:

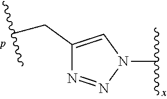 [SMILES: *Cc1cn(*)nn1]

wherein
   p indicates the bond to the co-polymer;
   x indicates the bond to the X₁ or X₂ group; and
X₁ and X₂ are each selected from:
   (i) —CH₂CH₂—[OCH₂CH₂]ₙ₁₀, wherein n10 is an integer from 2 to 10;
   (ii) —CH₂CH₂—[OCH₂CH₂]ₙ₁₁ wherein n11 is an integer from 2 to 10;
   (iii) —[CH₂CH₂O]ₙ₁₂—C(═O)—CH₂CH₂—C(═O), wherein n12 is an integer from 2 to 10; and
   (vi) —[CH₂CH₂O]ₙ₁₃—CH₂CH₂—NH—C(═O)—, wherein n13 is an integer from 2 to 10;
   (vii) —CH₂CH₂—[OCH₂CH₂]ₙ₁₄—O—C(═O), wherein n14 is an integer from 2 to 10; or
   (viii) —[CH₂CH₂O]ₙ₁₅—C(═O)—CH₂CH₂—C(═O), wherein n15 is an integer from 2 to 10.

Suitably, a1, a2 and b are as defined in any one of paragraphs (1) to (3) above. More suitably, a1, a2 and b are as defined in paragraph (2) or (3) above. Most suitably, a1, a2 and b are as defined in paragraph (3) above.

Suitably, W₁, W₂, X₁ and X₂ are as defined in any one of paragraphs (4) to (12) above.

More suitably, W₁, W₂, X₁ and X₂ are as defined in paragraph (6) to (10) above. Most suitably, W₁, W₂, X₁ and X₂ are as defined in paragraph (9) or (10) above.

Suitably, W₁ and W₂ are O.

In an embodiment:
a1, a2 and b are as defined in any one of paragraphs (1) to (3) above; and
W₁, W₂, X₁ and X₂ are as defined in any one of paragraphs (4) to (12) above.

In an embodiment:
a1, a2 and b are as defined in paragraph (2) or (3) above; and
W₁, W₂, X₁ and X₂ are as defined in paragraph (6) to (10) above.

In an embodiment:
a1, a2 and b are as defined in paragraph (3); and
W₁, W₂, X₁ and X₂ are as defined in paragraph (9) or (10) above.

Suitably, in the monomer of a catecholamine (e.g. dopamine) or DOPAC, that is covalently bound to a co-polymer of poly(ethylene oxide) and poly(propylene oxide), a1, a2, b, W₁, W₂, X₁ and X₂ may be as defined in paragraphs (1) to (12) above. Suitably, such monomers comprise a terminal catecholamine (e.g. dopamine) or DOPAC moeity bound to the copolymer optionally via the X₁ and X₂ groups.

Suitably, in the monomer of a catecholamine (e.g. dopamine) or DOPAC, that is covalently bound to a co-polymer of poly(ethylene oxide) and poly(propylene oxide), the catecholamine or DOPAC moiety will suitably be connected to the co-polymer or X₁ and X₂ via an appropriate bond, e.g. an amide or ester bond. Suitably, the catecholamine or DOPAC is selected from a dopamine, epinephrine L-DOPA or DOPAC moiety. Most suitably, the catecholamine is dopamine moiety.

### Functional Moieties

In certain embodiments of the invention, the polydopamine co-polymer nanoparticles of the present invention further comprise one or more functional moieties covalently attached or adsorbed to the nanoparticle, i.e. “loaded” to the nanoparticle.

Suitably, the functional moiety that is covalently attached or adsorbed to the nanoparticle is a moiety selected from one or more of:
(i) a pharmacologically active agent (e.g. a drug, biologic or neutraceutical);
(ii) a targeting ligand or targeting peptide (e.g. a receptor ligand, antibody or nanobody);
(iii) an imaging agent (e.g. a detectable moiety, such as a fluorophore, magnetic particles, radionuclides or a photoacoustic imaging agent).

Suitably, the functional moiety that is covalently attached or adsorbed to the nanoparticle is a moiety selected from one or more of:
(i) a pharmacologically active agent (e.g. a drug, biologic or neutraceutical),
(ii) a targeting ligand (e.g. a receptor ligand, antibody or nanobody),
(iii) an imaging agent (e.g. a detectable moiety, such as a fluorophore, magnetic particles, radionuclides or a photoacoustic imaging agent).

Suitably, the functional moiety that is covalently attached or adsorbed to the nanoparticle is a moiety selected from one or more of:
(i) a pharmacologically active agent (e.g. a drug, biologic or neutraceutical),
(ii) an imaging agent (e.g. a detectable moiety, such as a fluorophore, magnetic particles, radionuclides or a photoacoustic imaging agent).

### Pharmacologically Active Agents

In certain embodiments of the invention, the one or more functional moieties covalently attached or adsorbed to the polydopamine co-polymer nanoparticles is a pharmacologically active agent. Thus, the polydopamine co-polymer nanoparticles may be loaded with a pharmacologically active agent. The polydopamine co-polymer nanoparticles may be loaded with multiple pharmacologically active agents.

The pharmacologically active agent may be an agent which finds use in the treatment of cancer, diabetes, fungal infections, bacterial infections or autoimmune diseases.

The pharmacologically active agent may be a drug or a biologic.

The pharmacologically active agent may be attached to the polydopamine co-polymer nanoparticles via an appropriate linker group, e.g. a dye, glycol or a peptide.

In certain embodiments of the invention, the pharmacologically active agent is an anticancer agent.

Suitably, the anticancer agent is selected from one or more of:
a) Alkylating Agents, e.g. Altretamine, Bendamustine, Busulfan, Carmustine, Chlorambucil, Cyclophosphamide, Dacarbazine, Ifosfamide, Lomustine, Mechlorethamine, Melphalan, Procarbazine, Streptozocin, Temozolomide, Thiotepa, Trabectedin;
b) Platinum Coordination Complexes; e.g. Carboplatin, Cisplatin, Oxaliplatin
c) Antibiotics, (Cytotoxic Agents): e.g Bleomycin, Dactinomycin, Daunorubicin, Doxorubicin, Epirubicin, Idarubicin, Mitomycin, Mitoxantrone, Plicamycin, Valrubicin
d) Antimetabolites, e.g.:
   Antifolates: e.g. Methotrexate, Pemetrexed, Pralatrexate, Trimetrexate;
   Purine Analogues: e.g. Azathioprine, Cladribine, Fludarabine, Mercaptopurine, Thioguanine
   Pyrimidine Analogues: e.g. Azacitidine, Capecitabine, Cytarabine, Decitabine, Floxuridine, Fluorouracil, Gemcitabine, Trifluridine/Tipracil
e) Biologic Response Modifiers, e.g. Aldesleukin (IL-2), Denileukin Diftitox, Interferon Gamma
f) Histone Deacetylase Inhibitors; e.g. Belinostat, Panobinostat, Romidepsin, Vorinostat
g) Hormonal Agents; e.g.:
   Antiandrogens: such as Abiraterone, Apalutamide, Bicalutamide, Cyproterone, Enzalutamide, Flutamide, Nilutamide;
   Antiestrogens (including Aromatase Inhibitors): such as Anastrozole, Exemestane, Fulvestrant, Letrozole, Raloxifene, Tamoxifen, Toremifene;
   Gonadotropin Releasing Hormone Analogues: such as Degarelix, Goserelin, Histrelin, Leuprolide, Triptorelin; Peptide Hormones: such as Lanreotide, Octreotide, Pasireotide
h) Monoclonal Antibodies; e.g. Alemtuzumab, Atezolizumab, Avelumab, Bevacizumab, Blinatumomab, Brentuximab, Cemiplimab, Cetuximab, Daratumumab, Dinutuximab, Durvalumab, Elotuzumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Ramucirumab, Rituximab, Tositumomab, Trastuzumab;
i) Protein Kinase Inhibitors, e.g. Abemaciclib, Acalabrutinib, Afatinib, Alectinib, Alpelisib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Gefitinib, Gilteritinib, Glasdegib, Ibrutinib, Idelalisib, Imatinib, Ivosidenib, Ixazomib, Lapatinib, Larotrectinib, Lenvatinib, Lorlatinib, Midostaurin, Neratinib, Nilotinib, Niraparib, Olaparib, Osimertinib, Palbociclib, Pazopanib, Pexidartinib, Ponatinib, Regorafenib, Ribocicib, Rucaparib, Ruxolitinib, Selumetinib, Sonidegib, Sorafenib, Sunitinib, Talazoparib, Trametinib, Vandetanib, Vemurafenib, Vismodegib, Zanubrutinib
j) Taxanes, e.g. Cabazitaxel, Docetaxel, Paclitaxel
k) Topoisomerase Inhibitors; e.g. Etoposide, Irinotecan, Teniposide, Topotecan
l) Vinca Alkaloids e.g. Vinblastine, Vincristine, Vinorelbine
m) Miscellaneous anti cancer agents, e.g. Asparaginase (Pegaspargase), Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, Venetoclax.

Suitably, the pharmacologically active agent is an anticancer or antitumour agent which is used in the treatment of lung mesothelioma.

Suitably, the anticancer agent is selected from one or more of SN38 (a metabolite of irinotecan), nab-paclitaxel (Abraxane), 5-fluorouracil, leucovorin, irinotecan, oxaliplatin, doxorubicin, paclitaxel, gemcitabine and all trans retinoic acid (ATRA).

In a particular embodiment, the polydopamine co-polymer nanoparticles may be loaded with paclitaxel for use in the treatment of lung cancer, metastatic breast cancer and metastatic pancreatic cancer.

In certain embodiments of the invention, the polydopamine co-polymer nanoparticles may be used in the delivery ocular drugs in the treatment of retinal diseases, e.g. macular degeneration. Thus, the pharmacologically active agent may be an ocular drug.

In certain embodiments of the invention, the polydopamine co-polymer nanoparticles may be used in the delivery of DNA or RNA (including sRNA), for example in vaccines. Thus, the pharmacologically active agent may be selected from a nucleic acid such as DNA or RNA.

In certain embodiments of the invention, the polydopamine co-polymer nanoparticles may be used in the delivery of actives in the treatment of rheumatoid arthritis. Thus, the pharmacologically active agent may be an arthritis drug.

In certain embodiments of the invention, the polydopamine co-polymer nanoparticles may be used in the delivery of neutraceuticals in therapy. Thus, the pharmacologically active agent may be a neutraceutical.

### Imaging Agents

In certain embodiments of the invention, the one or more functional moieties covalently attached or adsorbed to the polydopamine nanoparticle is an imaging agent. The imaging agent may be a detectable moiety, such as a fluorophore, magnetic particles, a radionuclide or a photoacoustic imaging agent (e.g. cyanine dyes such indocyanine green (ICG)). Most suitably, the imaging agent is a fluorophore.

Polydopamine is known to complex metal ions, such as radionuclides which are used in radiotherapy. Suitably, a radionuclide may be bound to the polydopamine co-polymer nanoparticles and used in radiotherapy.

The imaging agent may attached to the polydopamine co-polymer nanoparticles via an appropriate linker group, e.g. e.g. a dye, glycol or a peptide.

### Targeting Ligands and Peptides

In certain embodiments of the invention, the one or more functional moieties covalently attached or adsorbed to the nanoparticle is a targeting ligand. The targeting ligand may be selected from a receptor ligand, antibody or nanobody.

In certain embodiments of the invention, the one or more functional moieties covalently attached or adsorbed to the nanoparticle is a targeting peptide.

In certain embodiments of the invention, the one or more functional moieties covalently attached or adsorbed to the nanoparticle is a drug-peptide conjugate, i.e. a drug molecule linked to a peptide, for example an ATRA conjugated peptide.

In certain embodiments of the invention, the one or more functional moieties covalently attached or adsorbed to the nanoparticle comprises ferulic acid.

In certain embodiments of the invention, the one or more functional moieties covalently attached or adsorbed to the nanoparticle comprises a pharmacologically active agent and ferulic acid. In such embodiments, the polydopamine co-polymer nanoparticles may find use in the delivery of active agents in therapy, for example:
the delivery of nucleic acids in therapy (e.g. DNA or RNA),
the delivery of nucleic acids (e.g. DNA or RNA) to human, animal or plant cells, or
the delivery of agents to plant cells.

In certain embodiments of the invention, the one or more functional moieties covalently attached or adsorbed to the nanoparticle is ferulic acid in combination with one or more pharmacologically active agents. The pharmacologically active agents may be any of those described herein, for example a nucleic acid or an anticancer agent.

The polydopamine co-polymer nanoparticles as defined herein may be loaded with a number of functional moieties which allow the delivery of a nucleic acid (e.g. DNA) to a cell, for example by immobilisation of DNA. Such functional moieties include poly-L-arginine and poly-L-histidine.

The polydopamine co-polymer nanoparticles as defined herein may be loaded with a peptide, for example peptides containing cysteine, lysine or any primary amine or thiol modified residues such as TAMRA-labelled-NLS peptide and drug-peptide conjugates such as ATRA conjugated peptides, or tumour penetrating peptides such as RGD peptides, or peptides which are cleavable in MMP.

In certain embodiments polydopamine co-polymer nanoparticles as defined herein may be functionalised with a drug-peptide conjugate, such as an ATRA conjugated peptide.

In certain embodiments polydopamine co-polymer nanoparticles as defined herein may be functionalised with a drug-peptide conjugate, such as an ATRA conjugated peptide, and an additional pharmacologically active agent such as a drug.

In certain embodiments polydopamine co-polymer nanoparticles as defined herein may be functionalised with a tumour penetrating peptide such as an RGD peptide.

In certain embodiments polydopamine co-polymer nanoparticles as defined herein may be functionalised with a tumour penetrating peptide, such as an RGD peptide, and an additional pharmacologically active agent such as a drug.

In certain embodiments polydopamine co-polymer nanoparticles as defined herein may be functionalised with a dye, for example as Rhodamine-TEG-NH₂ or Fluorescein.

In certain embodiments polydopamine co-polymer nanoparticles as defined herein may be functionalised with a dye, for example as Rhodamine-TEG-NH₂ or Fluorescein, an additional pharmacologically active agent such as a drug.

In certain cases, the loading of functional moieties may be achieved via covalent functionalisation of the pre-formed nanoparticles, for example by covalently binding a dye (such as Rhodamine-TEG-NH₂ or Fluorescein) or a peptide (e.g. peptides containing cysteine, lysine or any primary amine or thiol modified residues, such as TAMRA-labelled-NLS peptide and ATRA conjugated peptides, and tumour penetrating peptides such as iRGD) to the pre-formed nanoparticle.

In certain embodiments of the invention, the one or more functional moieties covalently attached or adsorbed to the nanoparticle may be a peptide.

The peptide may be an ATRA conjugated peptide or another MMP cleavable peptide, for example those disclosed on Table 9.2 on page 219 of Stimuli-responsive Drug Delivery Systems, 2018, edited by Amit Singh, Mansoor M Amij, which is incorporated herein by reference. Such peptides include may include one of the following sequences:

GLK;
(SEQ ID 1)
GPQGIFGQK
(SEQ ID 2)
GPVGLIGK
(SEQ ID 3)
GPLGIAGQ
(SEQ ID 4)
PLGLAG
(SEQ ID 5)
CPLGLAGG
(SEQ ID 6)
GPLGVRGDG
(SEQ ID 7)
VPLSLYSGCG
(SEQ ID 8)
GERGPPGPQGARGFZGTPGL
(SEQ ID 9)
CGPQGIWGQGCR
(SEQ ID 10)
PVGLIG
(SEQ ID 11)
RSWMGLP
(SEQ ID 12)
GPLGIAGQC
(SEQ ID 13)
PLGLAG
(SEQ ID 14)
AGFSGPLGMWSAGSFG

In other cases, the covalent attachment of a functional moiety to the nanoparticle may be achieved during the formation of the nanoparticle, e.g. by polymerising the catecholamine (e.g. dopamine) or DOPAC monomer components in the presence of a further catecholamine (e.g. dopamine) or DOPAC monomer that is covalently bound to a functional moiety. This results in the formation of nanoparticles with the required functional moiety or moieties covalently bound to them. The degree of loading can be controlled by controlling the proportion of the catecholamine (e.g. dopamine) or DOPAC monomer that is covalently attached to the functional moiety that is present in the monomer mixture.

The loaded functional moiety may have a cleavable link to the nanoparticle. Such a link may be cleavable under certain conditions, e.g. conditions found within the tumour microenvironment, within in a cell or in the presence of particular enzymes. The polydopamine co-polymer nanoparticles could be functionalised with peptides which are cleavable in the presence of matrix metalloproteinases (MMP). For example, the polydopamine co-polymer nanoparticles could be loaded with an ATRA conjugated peptide, which can be cleaved in the presence of MMP.

The loading of functional moieties to the nanoparticle may alternatively be achieved by covalent attachment of a functional moiety in between the polydopamine and the co-polymer. For example, the covalent attachment of a fluorescent group between the polydopamine and the co-polymer can be used as a means of incorporating fluorescence into the nanoparticle structure. Thus, the functional moiety may act as a linker group between the polydopamine and the co-polymer.

The loading of functional moieties to the polydopamine co-polymer nanoparticles of the invention may also be achieved by through non-covalent adsorption, for example anti-cancer drugs such as paclitaxel, gemcitabine and SN38 can be adsorbed onto the polydopamine co-polymer nanoparticles.

### Monomers and Synthesis

According to a further aspect of the invention there is provided a process for preparing polydopamine co-polymer nanoparticles as defined herein, the process comprising:
polymerising a catecholamine monomer (e.g. dopamine monomer) or DOPAC monomer with a monomer of a catecholamine (e.g. dopamine) or DOPAC that is covalently bound to a co-polymer of poly(ethylene oxide) and poly(propylene oxide) to form polydopamine co-polymer nanoparticles having a co-polymer of poly(ethylene oxide) and poly(propylene oxide) covalently bound thereto.

Suitably, the catecholamine monomer (e.g. dopamine monomer) or DOPAC monomer, that is covalently bound to a co-polymer of poly(ethylene oxide) and poly(propylene oxide) may be any of the monomers described herein.

In another aspect, the present invention also provides polydopamine co-polymer nanoparticles obtainable by, obtained by or directly obtained by a process as defined herein.

In another aspect, the present invention also provides polydopamine co-polymer nanoparticles formed from the polymerisation of:
1) a catecholamine monomer (e.g. dopamine monomer) or DOPAC monomer; with
2) a monomer of a catecholamine (e.g. dopamine) or DOPAC, that is covalently bound to a co-polymer of poly(ethylene oxide) and poly(propylene oxide).

Suitably, the monomer of a catecholamine (e.g. dopamine) or DOPAC, that is covalently bound to a co-polymer of poly(ethylene oxide) and poly(propylene oxide) may be any of the monomers described herein.

Suitably, in the monomer of a catecholamine (e.g. dopamine) or DOPAC that is covalently bound to a co-polymer of poly(ethylene oxide) and poly(propylene oxide), the co-polymer of poly(ethylene oxide) and poly(propylene oxide) is a tri-block co-polymer having a central block of poly(propylene oxide) flanked on each side by blocks of poly(ethylene oxide).

Suitably, in the monomer of a catecholamine (e.g. dopamine) or DOPAC that is covalently bound to a co-polymer of poly(ethylene oxide) and poly(propylene oxide), the co-polymer of poly(ethylene oxide) and poly(propylene oxide) has the formula:

-[poly(ethylene oxide)]-[poly(propylene oxide)]-[poly(ethylene oxide)]-

or

—X₁-[poly(ethylene oxide)]-[poly(propylene oxide)]-[poly(ethylene oxide)]-X₂—

where X₁ and X₂ are each independently a linker group that connects the co-polymer to the polydopamine or a detectable moiety (e.g. a fluorophore) that connects the co-polymer to the polydopamine.

Suitably, in the monomer of a catecholamine (e.g. dopamine) or DOPAC, that is covalently bound to a co-polymer of poly(ethylene oxide) and poly(propylene oxide), a1, a2, b, W₁, W₂, X₁ and X₂ may be as defined in paragraphs (1) to (12) above. Suitably, such monomers comprise a terminal catecholamine (e.g. dopamine) or DOPAC moiety bound to the copolymer optionally via the X₁ and X₂ groups.

Suitably, in the monomer of a catecholamine (e.g. dopamine) or DOPAC, that is covalently bound to a co-polymer of poly(ethylene oxide) and poly(propylene oxide), the catecholamine or DOPAC moiety will suitably be connected to the co-polymer or X₁ and X₂ via an appropriate bond, e.g. an amide or ester bond. Suitably, the catecholamine or DOPAC is selected from a dopamine, epinephrine L-DOPA or DOPAC moiety. Most suitably, the catecholamine is a dopamine moiety.

In an embodiment, the monomer of a catecholamine (e.g. dopamine) or DOPAC that is covalently bound to a co-polymer of poly(ethylene oxide) and poly(propylene oxide) has the formula:

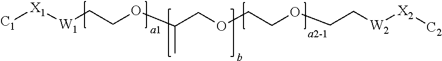

wherein:
W₁ and W₂ are selected from O or NH; or
W₁ and W₂ are selected from:

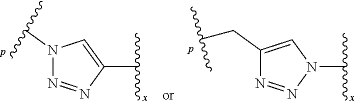 [SMILES: *c1cn(*)nn1 ; *Cc1cn(*)nn1]

wherein:
p indicates the bond to the co-polymer;
x indicates the bond to the X₁ or X₂ group,
a1 is an integer between 50 and 150;
a2 is an integer between 50 and 150;
b is an integer between 20 and 80; and
X₁ and X₂ are each independently:
   a linker group; or
   a detectable moiety (e.g. a fluorophore); and
C₁ and C₂ are a catecholamine or DOPAC.

The C₁ and C₂ groups will be connected to the X₁ or X₂ group via an appropriate bond, e.g. an amide or ester bond.

Suitably, C₁ and C₂ are selected from a dopamine, epinephrine L-DOPA or DOPAC moiety.

Suitably, W₁ and W₂ are selected from O or NH; and either:
i) X₁ and X₂ are each a group of the formula:

—C(═O)—[C H₂]ₙ—C(═O)—
wherein n is an integer from 1 to 10; or
ii) X₁ and X₂ are each a group of the formula:

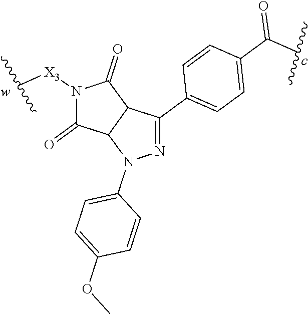

wherein:
w indicates the bond to the W₁ or W₂ group;
c indicates the bond to the C₁ or C₂ group;
X₃ is selected from:
   (i) —C(═O)—[CH₂]ₙ₁—. wherein n1 is an integer from 2 to 10;
   (ii) —[CH₂]ₙ₂—, wherein n2 is an integer from 2 to 10;
   (iii) —C(═O)—CH₂CH₂—[OCH₂CH₂]ₙ₃—, n3 is an integer from 2 to 10;
and
C₁ and C₂ are each a group of a formula selected from:

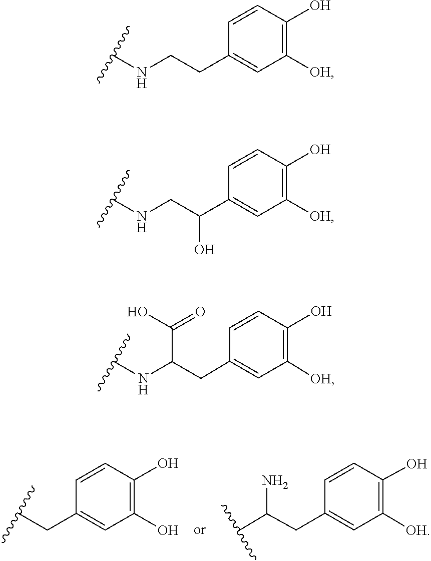 [SMILES: *NCCc1ccc(O)c(O)c1 ; *NCC(O)c1ccc(O)c(O)c1 ; *NC(Cc1ccc(O)c(O)c1)C(=O)O ; *Cc1ccc(O)c(O)c1 ; *C(N)Cc1ccc(O)c(O)c1]

Suitably, W₁ and W₂ are selected from O or NH; and either:
X₁ and X₂ are each a group of the formula:

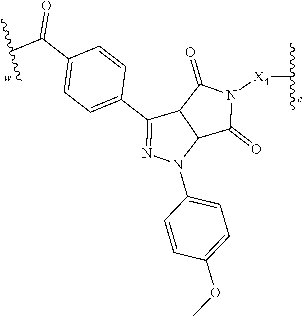

w indicates the bond to the W₁ or W₂ group;
c indicates the bond to the C₁ or C₂ group;
either
i) X₄ is selected from:
   (i) —[CH₂]ₙ₄—C(═O). wherein n4 is an integer from 2 to 10;
   (ii) —[CH₂]ₙ₅—, wherein n5 is an integer from 2 to 10;
   (iii) —[CH₂CH₂O]ₙ₆—CH₂CH₂—C(═O)—, wherein n6 is an integer from 2 to 10; and
   C₁ and C₂ are each a group of a formula selected from:

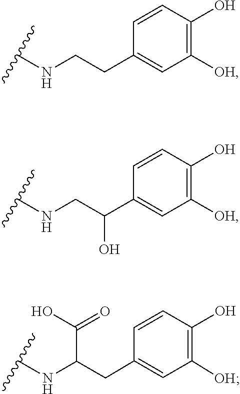 [SMILES: *NCCc1ccc(O)c(O)c1 ; *NCC(O)c1ccc(O)c(O)c1 ; *NC(Cc1ccc(O)c(O)c1)C(=O)O]

or
   ii) X₄ is selected from:
      (i) —[CH₂]ₙ₄—C(═O). wherein n4 is an integer from 2 to 10;
      (ii) —[CH₂]ₙ₅—C(═O), wherein n5 is an integer from 2 to 10;
      (iii) —[CH₂CH₂O]ₙ₆—CH₂CH₂—C(═O)—, wherein n6 is an integer from 2 to 10; and
   C₁ and C₂ are each a group of a formula selected from:

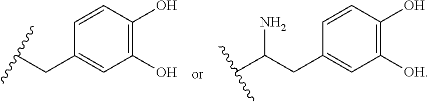 [SMILES: *Cc1ccc(O)c(O)c1 ; *C(N)Cc1ccc(O)c(O)c1]

Suitably, W₁ and W₂ are each a group of the formula:

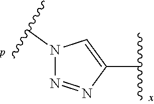 [SMILES: *c1cn(*)nn1]

wherein
p indicates the bond to the co-polymer;
x indicates the bond to the X₁ or X₂ group;
wherein either
i) X₁ and X₂ are each selected from:
(i) —CH₂—; or
(ii) —CH₂CH₂—[OCH₂CH₂]ₙ₇—, wherein n7 is an integer from 2 to 10; and C₁ and C₂ are a group of a formula selected from:

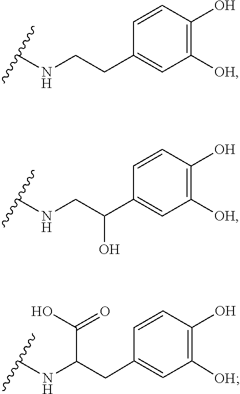 [SMILES: *NCCc1ccc(O)c(O)c1 ; *NCC(O)c1ccc(O)c(O)c1 ; *NC(Cc1ccc(O)c(O)c1)C(=O)O]

or
ii) X₁ and X₂ are each selected from:
(i) —CH₂—C(═O)—;
(ii) —CH₂CH₂—[OCH₂CH₂]ₙ₈—NH—C(═O)—, wherein n8 is an integer from 2 to 10; or
(iii) —CH₂CH₂—[OCH₂CH₂]ₙ₉—O—C(═O)—, wherein n9 is an integer from 2 to 10; and
C₁ and C₂ are a group of the formula:

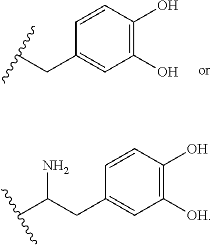 [SMILES: *Cc1ccc(O)c(O)c1 ; *C(N)Cc1ccc(O)c(O)c1]

Suitably, W₁ and W₂ are each a group of the formula:

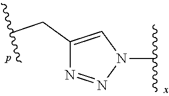 [SMILES: *Cc1cn(*)nn1]

wherein
   p indicates the bond to the co-polymer;
   x indicates the bond to the X₁ or X₂ group;
wherein either
i) X₁ and X₂ are each selected from:
   (i) —CH₂CH₂—[OCH₂CH₂]ₙ₁₀, wherein n10 is an integer from 2 to 10;
   (ii) —CH₂CH₂—[OCH₂CH₂]ₙ₁₁ wherein n11 is an integer from 2 to 10;
   (iii) —[CH₂CH₂O]ₙ₁₂—C(═O)—CH₂CH₂—C(═O), wherein n12 is an integer from 2 to 10; and
   C₁ and C₂ are selected from a group of the formula:

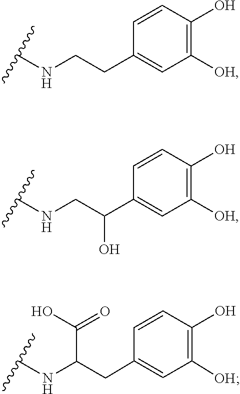 [SMILES: *NCCc1ccc(O)c(O)c1 ; *NCC(O)c1ccc(O)c(O)c1 ; *NC(Cc1ccc(O)c(O)c1)C(=O)O]

or
ii) X₁ and X₂ are each a group selected from:
   (i) —[CH₂CH₂O]ₙ₁₃—CH₂CH₂—NH—C(═O)—, wherein n13 is an integer from 2 to 10;
   (ii) —CH₂CH₂—[OCH₂CH₂]ₙ₁₄—O—C(═O), wherein n14 is an integer from 2 to 10;
   (iii) —[CH₂CH₂O]ₙ₁₅—C(═O)—CH₂CH₂—C(═O), wherein n15 is an integer from 2 to 10;
and
C₁ and C₂ are selected from a group of the formula:

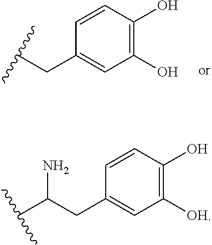 [SMILES: *Cc1ccc(O)c(O)c1 ; *C(N)Cc1ccc(O)c(O)c1]

Suitably, W₁ and W₂ are selected from O or NH; and
either
i) X₁ and X₂ are each a group of the formula:

—C(═O)—[C H₂]ₙ—C(═O)—
wherein n is an integer from 1 to 10; or
ii) X₁ and X₂ are each a group of the formula:

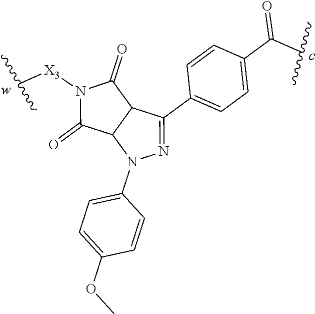

wherein:
w indicates the bond to the W₁ or W₂ group;
c indicates the bond to the C₁ or C₂ group; and
X₃ is selected from:
   (i) —C(═O)—[CH₂]ₙ₁—. wherein n1 is an integer from 2 to 10;
   (ii) —[CH₂]ₙ₂—, wherein n2 is an integer from 2 to 10; or
   (iii) —C(═O)—CH₂CH₂—[OCH₂CH₂]ₙ₃—, n3 is an integer from 2 to 10;
and C₁ and C₂ are each a group of the formula:

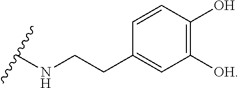 [SMILES: *NCCc1ccc(O)c(O)c1]

Suitably, W₁ and W₂ are selected from O or NH; and X₁ and X₂ are each independently:
a linker of the formula:

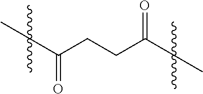 [SMILES: *C(=O)CCC(*)=O]

or a fluorophore of the formula:

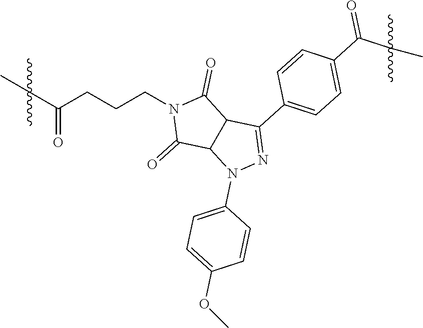 [SMILES: *C(=O)CCCN1C(=O)C2C(c3ccc(C(*)=O)cc3)=NN(c3ccc(OC)cc3)C2C1=O]

wherein the fluorophore may be attached to the C₁ or C₂ group at either vacant bond; and C₁ and C₂ are each a group of the formula:

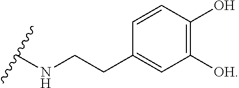 [SMILES: *NCCc1ccc(O)c(O)c1]

Suitably, the monomer of a catecholamine (e.g. dopamine) or DOPAC that is covalently bound to a co-polymer of poly(ethylene oxide) and poly(propylene oxide) has the formula:

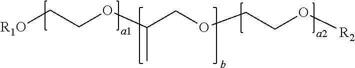

wherein
a1 is an integer between 50 and 150;
a2 is an integer between 50 and 150;
b is an integer between 20 and 80; and
R₁ is a group of the formula:

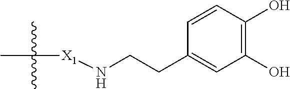

wherein X₁ is a linker group or a detectable moiety (e.g. a fluorophore); and R₂ is a group of the formula:

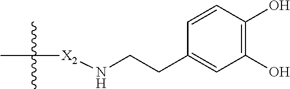

wherein X₂ is a linker group or a detectable moiety (e.g. a fluorophore).

Suitably, X₁ and X₂ are as defined herein.

Suitably, X₁ and X₂ are each independently a linker of the formula:

—C(═O)—[CH₂]ₙ—C(═O)—

wherein n is an integer from 1 to 10;
or a detectable moiety (e.g. a fluorophore).

Suitably, X₁ and X₂ are each independently selected from:
(i) a linker of the formula:

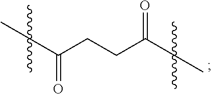 [SMILES: *C(=O)CCC(*)=O]

(ii) a fluorophore of the formula:

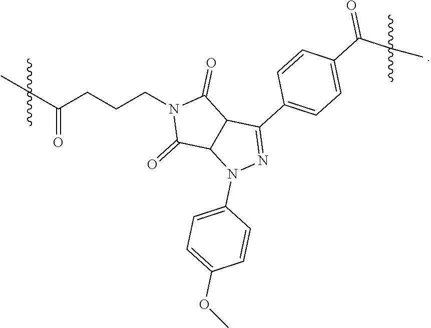 [SMILES: *C(=O)CCCN1C(=O)C2C(c3ccc(C(*)=O)cc3)=NN(c3ccc(OC)cc3)C2C1=O]

Suitably, C₁ and C₂ are a dopamine moiety:

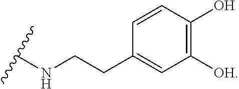 [SMILES: *NCCc1ccc(O)c(O)c1]

Suitably,
(i) a1 is an integer between 75 and 125;
   a2 is an integer between 75 and 125; and
   b is an integer between 25 and 75.

More suitably,
(ii) a1 is an integer between 90 and 110;
   a2 is an integer between 90 and 110; and
   b is an integer between 45 and 65.

Suitably, a1 and a2 are the same.

Suitably, a1 is 101; a2 is 101; and b is 56 (i.e. Pluronic F127).

Suitably, the polymerisation of the monomers is conducted in a solvent comprising a mixture of ethanol and water. More suitably, the solvent is selected from:
(i) water comprising 0 to 60% v/v ethanol;
(ii) water comprising 5 to 45% v/v ethanol;
(iii) water comprising 5 to 40% v/v ethanol;
(iv) water comprising 10 to 35% v/v ethanol; or
(v) water comprising 10 to 20% v/v ethanol.

Suitably, the molar ratio of catecholamine (e.g. dopamine) or DOPAC monomer to monomers of catecholamine (e.g. dopamine) or DOPAC that are covalently bound to a co-polymer of poly(ethylene oxide) and poly(propylene oxide) is selected from:
(i) 5:1 to 150:1;
(ii) 5:1 to 100:1;
(iii) 5:1 to 80:1;
(iv) 5:1 to 75:1;
(v) 5:1 to 50:1;
(vi) 10:1 to 40:1;
(vii) 10:1 to 30:1;
(viii) 15:1 to 25:1; or
(ix) 20:1.

Suitably, the polymerisation reaction is conducted in the presence of a suitable base (e.g. trizma base).

Suitably, the polymerisation reaction is conducted at a temperature of between 5 to 35° C., optionally between 15 and 25° C. or 20 and 25° C.

Suitably, the process further comprises a step of collecting the polydopamine co-polymer nanoparticles formed by the process, optionally by centrifugation, filtration and/or dialysis.

Suitably, the process further comprises a step of washing the collected nanoparticles. The washing may comprise several steps of resuspending the collected nanoparticles in a suitable vehicle and recollecting the particles by centrifugation.

Suitably, the process further comprises:
a) adding an additional monomer of dopamine covalently attached to functional moiety to the mixture of the catecholamine (e.g. dopamine) or DOPAC monomer and the monomer of a catecholamine (e.g. dopamine) or DOPAC that is covalently bound to a co-polymer of poly(ethylene oxide) and poly(propylene oxide) and polymerising the monomers to form polydopamine co-polymer nanoparticles having a co-polymer of poly(ethylene oxide) and poly(propylene oxide) and a functional moiety covalently bound thereto; or
b) covalently attaching a functional moiety to the polydopamine co-polymer nanoparticles formed by the process defined herein; and/or
c) adsorbing a functional moiety to the polydopamine co-polymer nanoparticles formed by the process defined herein.

### In Situ Covalent Attachment

In certain embodiments, the process further comprises adding an additional monomer of dopamine that is covalently attached to a functional moiety to the mixture of the catecholamine (e.g. dopamine) or DOPAC monomer and the monomer of a catecholamine (e.g. dopamine) or DOPAC that is covalently bound to a co-polymer of poly(ethylene oxide) and poly(propylene oxide), and polymerising the monomers to form polydopamine co-polymer nanoparticles comprising polydopamine having a co-polymer of poly(ethylene oxide) and poly(propylene oxide) and a functional moiety covalently bound thereto.

The loading of functional moieties may therefore be achieved through covalent attachment of the functional moiety during the formation of the nanoparticle, by polymerising in the presence of a further functionalised catecholamine (e.g. dopamine) or DOPAC monomer covalently attached to a functional moiety as defined herein. Thus, in this embodiment, the process comprises polymerising three monomers:
(i) catecholamine (e.g. dopamine) or DOPAC monomer;
(ii) a monomer of a catecholamine (e.g. dopamine) or DOPAC that is covalently bound to a co-polymer of poly(ethylene oxide) and poly(propylene oxide); and
(iii) a monomer of dopamine covalently attached to a functional moiety as defined herein (e.g. ATRA-modified with dopamine);
   to form polydopamine co-polymer nanoparticles comprising polydopamine having a co-polymer of poly(ethyleneoxide) and poly(propylene oxide) and a functional moiety covalently bound thereto.

### In Situ Covalent Attachment as a Linker

In certain embodiments, the loading of functional moieties may also be achieved by covalent attachment of a functional moiety between the polydopamine and the co-polymer, for example the covalent attachment of a fluorescent group (e.g. a fluorophore) between the polydopamine and co-polymer of poly(ethylene oxide) and poly(propylene oxide) enables the fluorophore to be incorporated into the polydopamine co-polymer nanoparticle structure during polymerisation. Thus, the functional moiety is present as a linker group between the polydopamine and co-polymer.

### Covalent Attachment to Pre-formed Nanoparticle

In certain embodiments, the process further comprises covalently attaching a functional moiety to the polydopamine co-polymer nanoparticles formed by the process defined herein. Non-limiting examples of functional moieties which may be attached in this manner include a dye (such as Rhodamine-TEG-NH₂ or Fluorescein), a drug molecule, a small peptide or drug peptide-conjugate. Non-limiting examples include peptides containing cysteine, lysine or any primary amine or thiol modified residues, such as TAMRA-labelled-NLS peptide, drug-peptide conjugates such as ATRA conjugated peptides, and tumour penetrating peptides such as the RGD peptide family.

### Adsorption to Pre-formed Nanoparticle

In certain embodiments, the process further comprises adsorbing a functional moiety to the polydopamine co-polymer nanoparticles formed by the process defined herein. Non-limiting examples of functional moieties which may be adsorbed in this manner include anti-cancer drugs such as paclitaxel, gemcitabine and SN38.

### Pharmaceutical Compositions

In another aspect, there is provided a pharmaceutical composition comprising polydopamine co-polymer nanoparticles of the invention as defined hereinbefore and one or more pharmaceutically acceptable excipients.

Suitably, the polydopamine co-polymer nanoparticles are loaded with a functional moiety defined herein, which is either covalently attached or adsorbed to the nanoparticle.

It is anticipated that the polydopamine co-polymer nanoparticles of the present invention could be loaded with a wide variety of different functional moieties, depending on the required application, including but not limited to the functional moieties described hereinbefore.

Suitably, the functional moiety is pharmacologically active agent, such as a drug or biologic.

The pharmacologically active agent may be selected from one or more of:
a) an anticancer agent;
b) an antidiabetic agent;
c) an antifungal agent;
d) an antibacterial agent;
e) an imaging agent.

The pharmacologically active agent may be an anticancer agent, optionally selected from those disclosed herein.

Thus, according to an embodiment of the invention, there is provided a pharmaceutical composition which comprises:
polydopamine co-polymer nanoparticles of the invention as defined hereinbefore,
an anticancer agent, and
one or more pharmaceutically acceptable excipients.

The anticancer agent may be selected from any of those described herein.

In certain embodiments, the functional moiety is an imaging agent. The imaging agent may be selected from a fluorophore, magnetic particles, a radionuclide, a photoacoustic imaging agent (e.g. cyanine dyes such indocyanine green (ICG)) or a photothermal agent.

Thus, according to an embodiment of the invention, there is provided a pharmaceutical composition which comprises:
polydopamine co-polymer nanoparticles of the invention as defined hereinbefore,
an imaging agent, and
one or more pharmaceutically acceptable excipients.

Suitably, the pharmaceutical composition is dispersed in an aqueous vehicle.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

In using polydopamine co-polymer nanoparticles of the invention for therapeutic or prophylactic purposes it will generally be administered so that a daily dose in the range, for example, 0.1 mg/kg to 75 mg/kg body weight of imaging agent or pharmacologically active agent is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of polydopamine co-polymer nanoparticles of this invention.

### Therapeutic Uses and Applications

### Therapy

In another aspect, the present invention provides polydopamine co-polymer nanoparticles as defined herein, for use in therapy. The present invention also provides a pharmaceutical composition as defined herein, for use in therapy.

In another aspect, the present invention provides polydopamine co-polymer nanoparticles as defined herein, wherein the polydopamine co-polymer nanoparticles are loaded with a functional moiety as defined herein, or a pharmaceutical composition comprising said nanoparticles, for use in therapy.

### Cancer

Suitably, the functional moiety is a pharmacologically active agent (e.g. a drug or biologic). More suitably, the pharmacologically active agent is an anticancer agent and the polydopamine co-polymer nanoparticles are for use in the treatment of cancer.

Thus, the present invention provides polydopamine co-polymer nanoparticles as defined herein, wherein the polydopamine co-polymer nanoparticles are loaded with an anticancer agent, or a pharmaceutical composition comprising said nanoparticles, for use in the treatment of cancer.

In another aspect, the present invention provides the use of polydopamine co-polymer nanoparticles as defined herein, or a pharmaceutical composition as defined herein, wherein the polydopamine co-polymer nanoparticles are loaded with an anticancer agent, in the manufacture of a medicament for use in the treatment of cancer.

The present invention provides a method of treating cancer in a patient in need of such treatment, said method comprising administering to said patient a therapeutically effective amount of polydopamine co-polymer nanoparticles as defined herein, wherein the polydopamine co-polymer nanoparticles are loaded with an anticancer agent, or a pharmaceutical composition comprising said nanoparticles.

Suitably, the cancer is human cancer. Suitably, the cancer is a solid tumour.

Suitably the cancer is selected from pancreatic cancer, mesothelioma, bladder cancer, breast cancer, cervical cancer, colon & rectal cancer, endometrial cancer, kidney cancer, lip & oral cancer, liver cancer, melanoma, non-small cell lung cancer, nonmelanoma skin cancer, oral cancer, ovarian cancer, prostate cancer, sarcoma, small cell lung cancer, and thyroid cancer.

The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

In a particular embodiment, the pharmacologically active agent is an anticancer agent or antitumour agent, and the polydopamine co-polymer nanoparticles are for use in the delivery of chemotherapies in the treatment of lung mesothelioma.

The present invention provides polydopamine co-polymer nanoparticles as defined herein, wherein the polydopamine co-polymer nanoparticles are functionalised with an RGD peptide (e.g. iRGD or cRGD), or a pharmaceutical composition comprising said nanoparticles, for use in the treatment of cancer. The cancer may be selected from pancreatic cancer, breast cancer, lung cancer and glioblastoma.

In another aspect, the present invention provides the use of polydopamine co-polymer nanoparticles as defined herein in the manufacture of a medicament for use in the treatment of cancer, wherein the polydopamine co-polymer nanoparticles are loaded with an RGD peptide (e.g. iRGD or cRGD).

### Photothermal Therapy

In another aspect, the present invention provides polydopamine co-polymer nanoparticles as defined herein, for use in photothermal therapy. The present invention also provides a pharmaceutical composition as defined herein, for use in photothermal therapy.

In another aspect, the present invention provides the use of polydopamine co-polymer nanoparticles as defined herein in the manufacture of a medicament for use as a photothermal therapeutic agent.

The present invention provides a method of photothermal therapy, said method comprising administering to a patient in need of such treatment an effective amount of the polydopamine co-polymer nanoparticles as defined herein, or a pharmaceutical composition as defined herein, or a pharmaceutical composition comprising said nanoparticles.

In an embodiment, the photothermal therapy is used for the treatment of cancer, such as those defined above.

### Imaging

Suitably, the functional moiety is an imaging agent. Thus, the present invention provides polydopamine co-polymer nanoparticles as defined herein, wherein the polydopamine co-polymer nanoparticles are loaded with an imaging agent, or a pharmaceutical composition comprising said nanoparticles, for use as:
(i) a photoacoustic imaging agent;
(ii) a magnetic resonance imaging agent; or
(iii) a positron emission tomography (PET) imaging agent.

Thus, the present invention provides polydopamine co-polymer nanoparticles as defined herein, wherein the polydopamine co-polymer nanoparticles are loaded with an imaging agent, or a pharmaceutical composition comprising said nanoparticles, for use in:
(i) a method of photoacoustic imaging;
(ii) a method of magnetic resonance imaging; or
(iii) positron emission tomography (PET) scanning.

In an embodiment, the imaging agent is a detectable moiety, such as a fluorophore, magnetic particles, a radionuclide, a photoacoustic imaging agent (e.g. cyanine dyes such indocyanine green (ICG)). Most suitably, the imaging agent is a fluorophore.

In an embodiment, if the imaging agent is a magnetic particle, the polydopamine co-polymer nanoparticles of the present invention may find use as a contrast agent in magnetic resonance imaging (MRI).

In an embodiment, if the imaging agent is a photoacoustic imaging agent (e.g. cyanine dyes such indocyanine green (ICG), the polydopamine co-polymer nanoparticles of the present invention may find use in photoacoustic imaging.

In an embodiment, if the imaging agent is a radionuclide, the polydopamine co-polymer nanoparticles of the present invention may find use in positron emission tomography (PET) scanning or radiotherapy.

### Transfection

The polydopamine co-polymer nanoparticles of the present invention may find use in transfection of animal (e.g. human) or plant cells. Suitably, the functional moiety is a poly(amino acid) which is positively charged at pH 7, e.g. a poly(amino acid) selected from poly-L-arginine and poly-L-histidine, or a combination thereof. Nanoparticles modified with poly(amino acid) such as poly-L-arginine and poly-L-histidine may be utilised in the delivery of nucleic acids, e.g. DNA or RNA.

Thus, there is provided polydopamine co-polymer nanoparticles as defined herein, for use in the delivery of a nucleic acid molecule into a target cell, wherein the polydopamine co-polymer nanoparticles are loaded with one or more functional moieties which allow the delivery of nucleic acids to a cell. Suitably, the functional moieties comprise one or more of poly-L-arginine and poly-L-histidine.

There is also provided a method of delivering a nucleic acid into a target cell, said method comprising contacting said target cell with polydopamine co-polymer nanoparticles as defined herein, wherein the polydopamine co-polymer nanoparticles are loaded with one or more functional moieties which allow the delivery of nucleic acids to a cell. Suitably, the functional moieties comprise one or more of poly-L-arginine and poly-L-histidine.

### Other Therapeutic Uses and Applications

The polydopamine co-polymer nanoparticles as defined herein may be loaded with a number of functional moieties and find use in a wide variety of therapeutic applications.

Particular examples include:
the delivery of DNA (e.g. plasmid DNA) and RNA (including siRNA) for use in therapy. For example, polydopamine co-polymer nanoparticles loaded with a mixture of poly-L-arginine and poly-L-histidine may be used in such an application.
the delivery of ocular drugs in the treatment of retinal diseases, for example macular degeneration.
the delivery of nutraceuticals, such as the delivery of vitamins and other compounds that aid the health of organism (see for example Begines et al, Nanomaterials 2020, 10, 1403; doi:10.3390/nano10071403), for example:
   delivery of flavonoids such as quercetin and others that are known to be potent antioxidants and implicated in removal of senescence,
   delivery of hypericin and emodin,
   delivery of compounds that can help in treating iron deficiency.

The polydopamine co-polymer nanoparticles as defined herein may be loaded with a number of functional moieties and find use in a wide variety of therapeutic applications. Thus, the polydopamine co-polymer nanoparticles may find use in the delivery of actives for use in the treatment of:
rheumatoid arthritis, Crohn's disease, rheumatoid arthritis, psoriatic arthritis and ankylosing spondylitis (for example Cimzia);
Acute lymphoblastic leukaemia (for example oncaspar);
Multiple sclerosis (for example Copaxone or Plegridy);
Hepatitis C infection (for example Peglntron);
Chemotherapy induced neutropenia (e.g. Neulasta);
Prostate cancer (for example Eligard);
Haemophilia (for example Adynovate);
Lung cancer, metastatic breast cancer or metastatic pancreatic cancer (for example Abraxane).

In certain embodiments, where the polydopamine co-polymer nanoparticles are for use in the delivery of active agents in therapy, they may also be functionalised with ferulic acid, in addition to the active agent.

The polydopamine co-polymer nanoparticles as defined herein may find use in the delivery of active agents to plant cells. In such embodiments, the polydopamine co-polymer nanoparticles may be functionalised with ferulic acid, in addition to the active agent.

Thus, there is provided polydopamine co-polymer nanoparticles as defined herein, functionalised with ferulic acid.

There is also provided polydopamine co-polymer nanoparticles as defined herein, functionalised with ferulic acid and any of the functional moieties defined herein.

There is also provided polydopamine co-polymer nanoparticles as defined herein, functionalised with ferulic acid and a nucleic acid.

The polydopamine co-polymer nanoparticles as defined herein may find use in the delivery of nucleic acids to plant cells (i.e. transfection). The use may be for the production of recombinant proteins. In such embodiments, the polydopamine co-polymer nanoparticles may be functionalised with one or more of ferulic acid or poly-L-arginine and poly-L-histidine.

### Routes of Administration

The polydopamine co-polymer nanoparticles of the invention or pharmaceutical compositions comprising these compounds may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

When loaded with an anticancer agent for cancer therapy or used as a photothermal therapy for cancer treatment, the polydopamine co-polymer nanoparticles of the present invention, or a pharmaceutical composition as defined herein, may be administered as a sole therapy or in combination with other treatment approaches, including conventional surgery, radiotherapy and/or chemotherapy. Such chemotherapy may include one or more of the following categories of anti-tumour agents:—
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (Cl 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the polydopamine co-polymer nanoparticles of this invention within the dosage range described hereinbefore and the other pharmaceutically-active agent within its approved dosage range.

According to this aspect of the invention there is provided a combination for use in the treatment of a cancer (for example a cancer involving a solid tumour) comprising polydopamine co-polymer nanoparticles of the invention or a pharmaceutical composition as defined hereinbefore, and another anti-tumour agent.

According to this aspect of the invention there is provided a combination for use in the treatment of a proliferative condition, such as cancer (for example a cancer involving a solid tumour), comprising polydopamine co-polymer nanoparticles of the invention or a pharmaceutical composition as defined hereinbefore, and any one of the anti-tumour agents listed herein above.

In a further aspect of the invention there is provided polydopamine co-polymer nanoparticles of the invention or a pharmaceutical composition as defined hereinbefore, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

### EXAMPLES

### Part 1

Here, we described the design of novel size-tuneable melanin-mimetic nanocarriers with covalently attached Pluronic F127 (F127@PDA) for drug delivery in solid tumour cells pancreatic cancer cells. Besides employing a highly reproducible synthesis method under mild conditions, they showed stability in physiological conditions, could further be functionalized and loaded with irinotecan prodrug SN38, used in the treatment of pancreatic cancer and other solid tumour cancers. Viability, cellular uptake and cytokine profiling studies of differently sized nanocarriers (40, 60 and 100 nm) demonstrated high bio- and immune-compatibility for all studied sizes. Four pancreatic cancer cell lines (AsPC-1, BxPC-3, MIA PaCa-2 and PANC-1) with different morphological and phenotypic characteristics were used as an in vitro model to assess biocompatibility, cellular uptake and drug release. Exposure to the drug encapsulated within the carrier was associated with an increased antiproliferative effect when compared to the free drug in all tested cell lines.

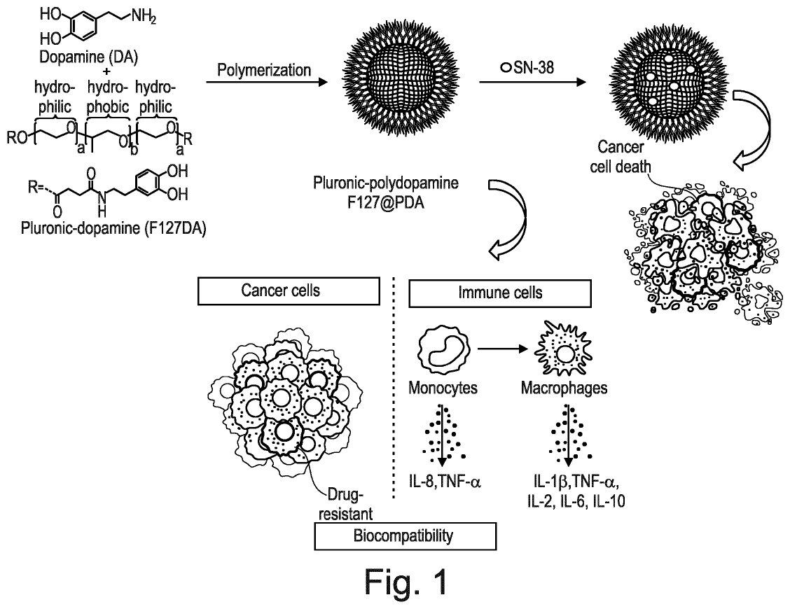

In this study polymer NPs were designed containing Pluronic as an alternative to PEG. Pluronic is an amphiphilic triblock co-polymer composed of two hydrophilic polyethylene oxide (PEO) blocks by a hydrophobic polypropylene oxide (PPO) segment (FIG. 1). Versatility in the chemical composition and simple introduction of various functional groups make Pluronic polymers a promising alternative to PEG in biomedical applications. Based on the PEO/PPO ratio, physical and chemical properties of the block co-polymer can be precisely adjusted, influencing its interactions with cells.^{[33,34]} Like PEG, the addition of Pluronic to nanoparticles (NPs) leads to a net-neutral and hydrophilic surface, which can facilitate penetration through the tumour ECM.^{[35,36]} Due to the presence of collagen, glycosaminoglycans and fibronectin, ECM has a net-negative charge and influences the transport through electrostatic and size-based interactions.^{[37]} The interest in the amphiphilic polymer was further increased with phase Ill clinical trials of doxorubicin-encapsulated Pluronic L61 and F127 micelles (SP1049C) for treatment of gastric and oesophagus cancer.^{[38]} Furthermore, Pluronic polymers have been shown to be effective sensitisers in multidrug resistant (MDR) cancer cells through different mechanisms, such as inhibition of the drug efflux transporter P-glycoprotein and micro-viscosity modification of the cellular membrane.^{[39]}

Herein, the reproducible synthesis of size-tuneable Pluronic F127-polydopamine (F127@PDA) nanocarriers is reported (FIG. 1). The size of the nanocarriers can easily be adjusted without affecting the composition of the NPs by changing the solvent ratio (ethanol/water) in the reaction mixture. The nanocarrier not only exhibited excellent colloidal stability but could be easily functionalized and loaded with irinotecan prodrug SN38. To evaluate the carrier system for treatment of pancreatic cancer, in vitro studies were conducted using four morphologically and genetically different pancreatic cell lines (AsPC-1, BxPC-3, MIA PaCa-2 and PANC-1), addressing the heterogeneity of pancreatic cancer. Different sizes (40, 60 and 100 nm) of nanocarriers were explored in terms of cell uptake and immunocompatibility. Although there are several reports describing the use of surfactants such as Pluronic F127 as templating agents^{[40,41]} in the synthesis of porous polydopamine NPs, to our knowledge this is the first report demonstrating the use of hybrid Pluronic F127 functionalized polydopamine nanocarriers.

### Results and Discussion

### Synthesis and Characterization of F127@PDA

Biopolymers are commonly used as smart drug delivery systems, due to their low toxicity, favourable cellular interactions, physicochemical versatility and ability to design various nanostructures with tuneable size and different surface properties.^{[42,43]} Melanin-like polymers have been shown to be highly biocompatible, act as radical scavengers and neuroprotection agents,^{[44-46]} as well as possessing excellent photothermal^{[47-51]} and photoacoustict^{5[2-55]} properties, all of which makes them a particularly promising candidate for drug delivery and diagnostics.

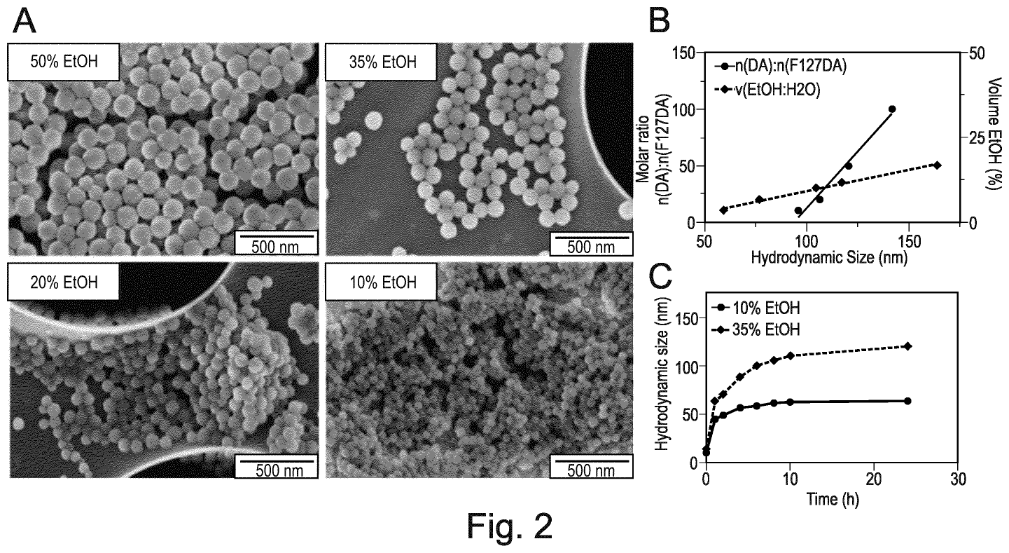

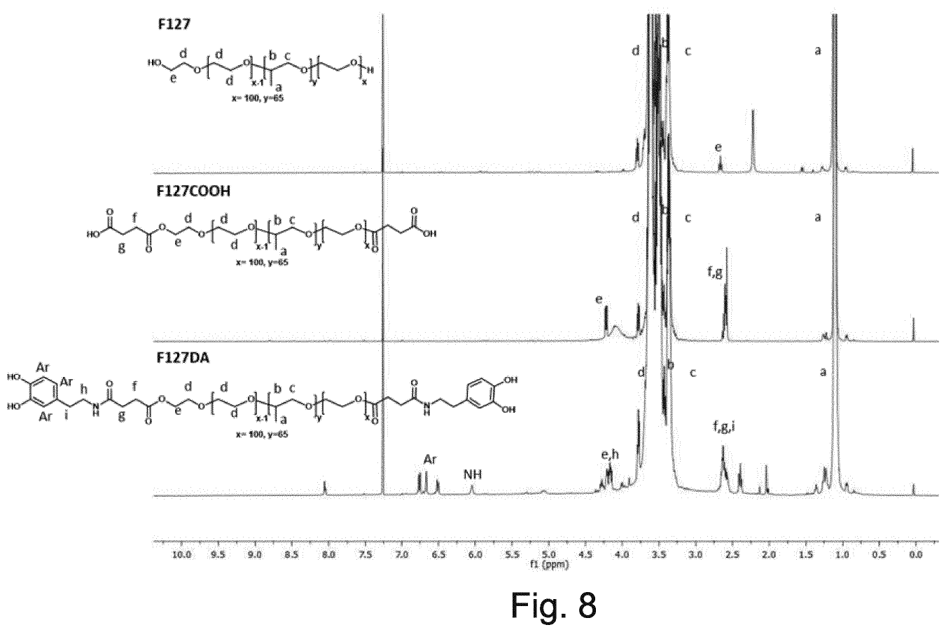

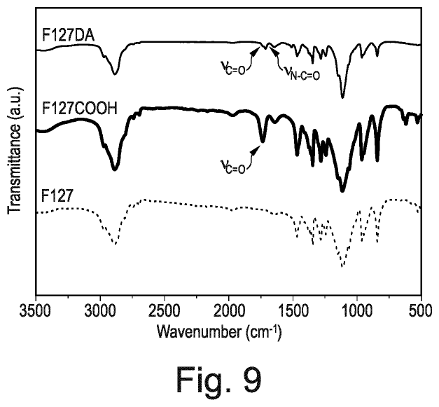

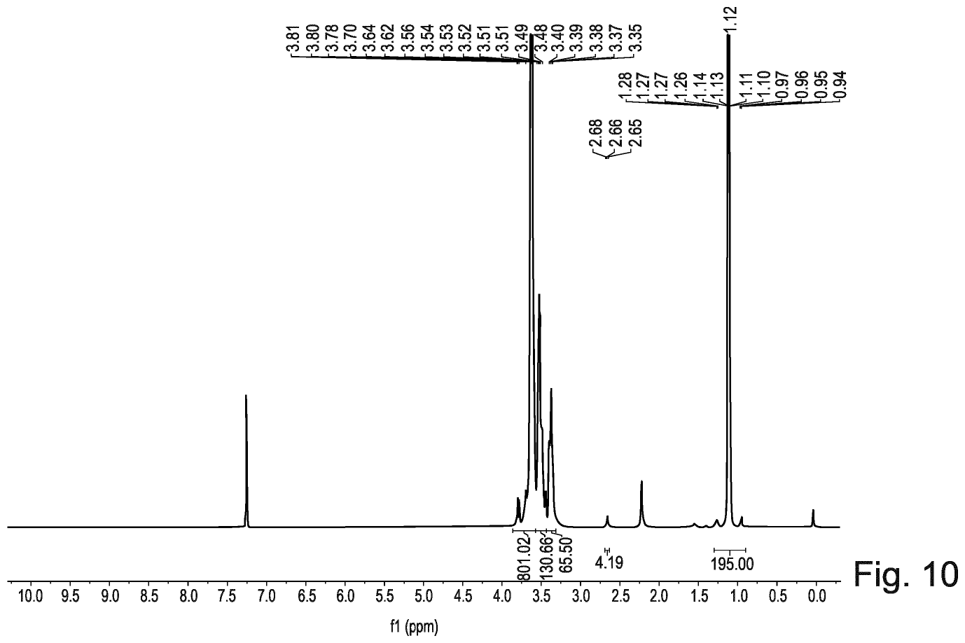

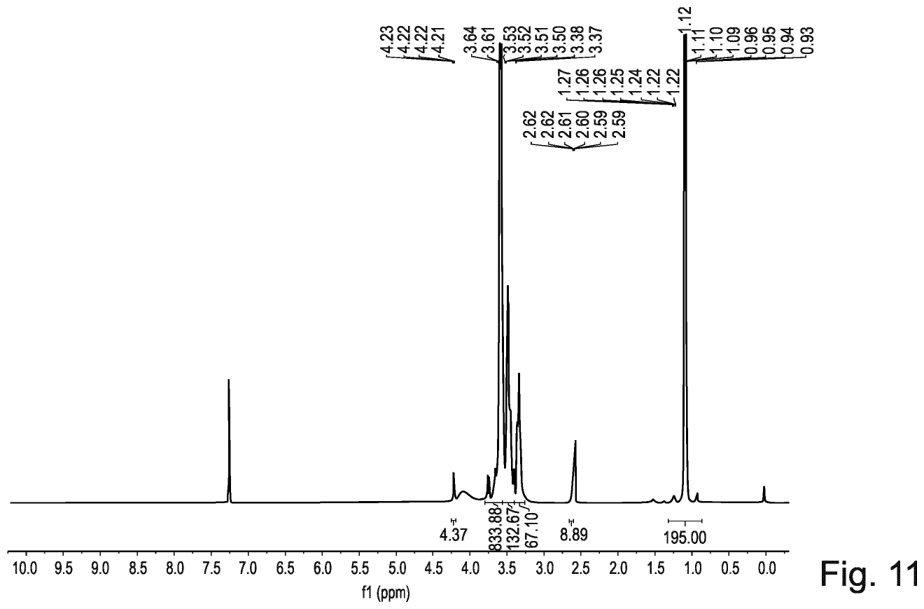

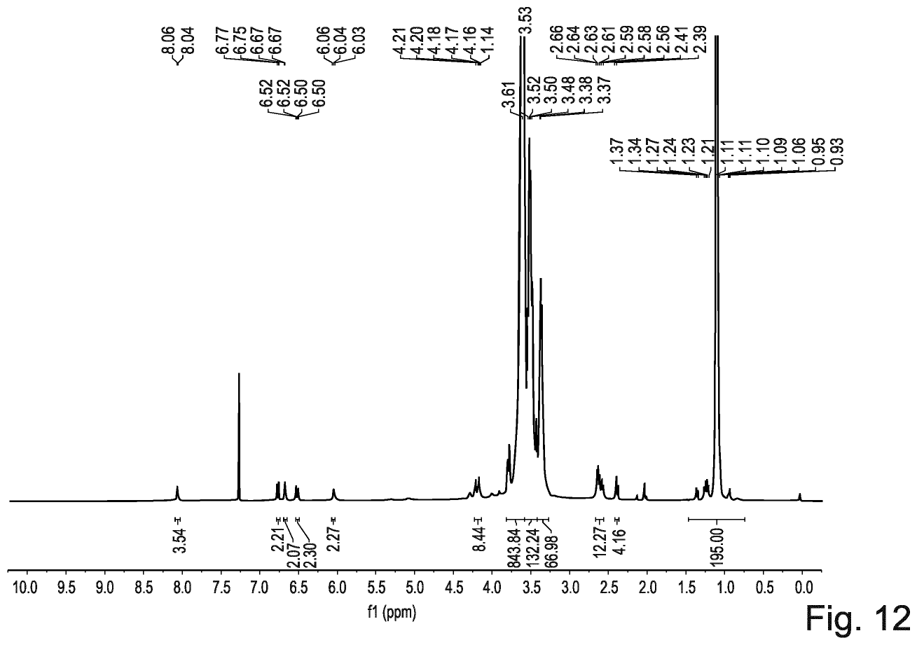

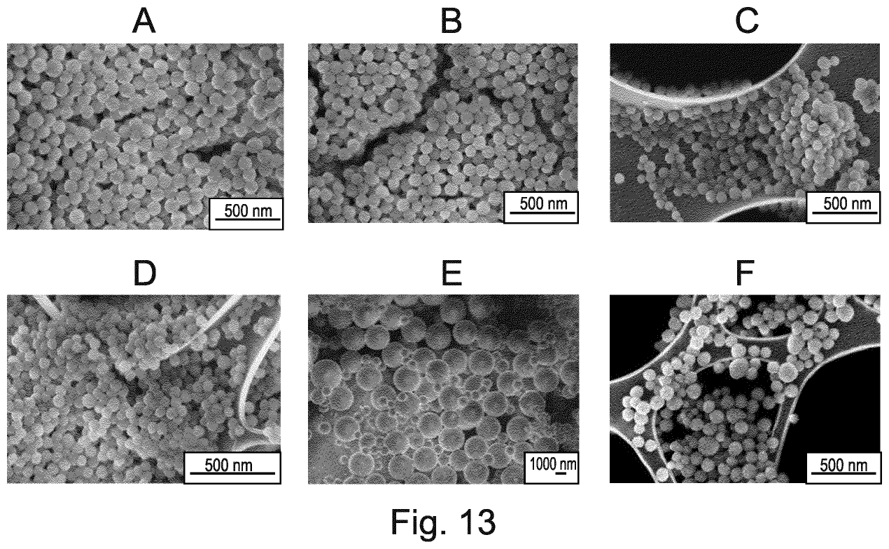

Melanin-mimetic F127@PDA NPs were prepared by oxidation and self-co-polymerization of dopamine hydrochloride (DA) and Pluronic F127-dopamine (F127DA) monomer. In order to have better control over the polymerization, the F127DA monomer was first synthesized through modification of the hydroxyl groups into carboxy-terminated Pluronic^{[56]} and coupling to DA, followed by structural characterisation, using ¹H NMR and FT-IR (Scheme S1 and FIG. 8-12, see Supporting Information (SI)). The formation of F127@PDA NPs was achieved by co-polymerization of F127DA and DA in mild reaction conditions (water and ethanol mixture with Tris base). Based on the reaction conditions spherical nanoparticles were obtained in a range from 40±5.4 to 100±14.9 nm (FIG. 2, FIG. 13 and Table S1, SI) with a zeta potential from −4 to −19 mV, which is similar to the zeta potential of F127 micelles^{[57]} and ultrasmall PEG-polydopamine NPs.^{[58]} Those findings indicate the successful coating of F127@PDA NPs with Pluronic, as the surface/zeta potential of bare PDA was found to be approximately −40 mV.^{[59]}

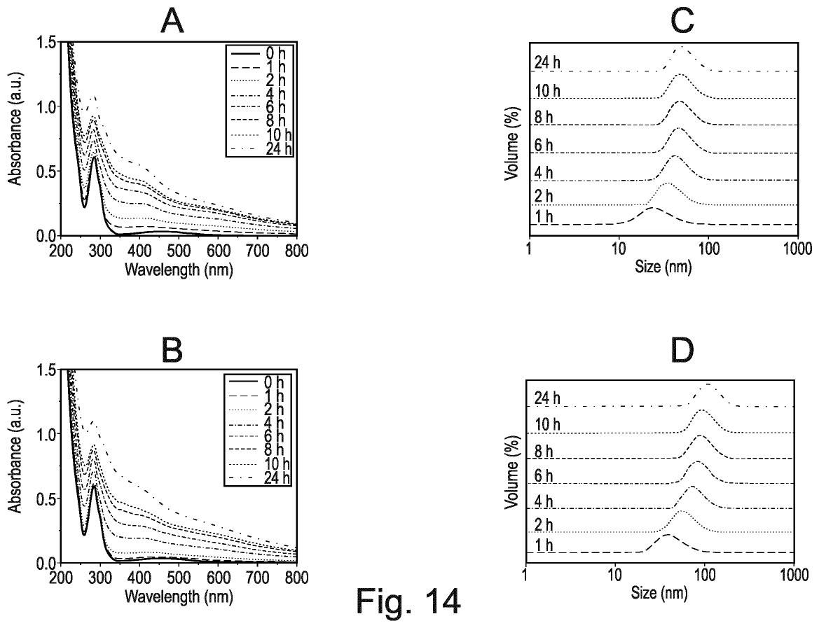

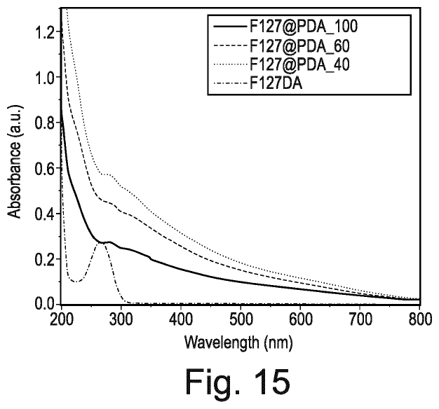

To further elucidate the influence that ethanol has during the co-polymerization of F127@PDA NPs, in situ particle formation was studied by measuring the hydrodynamic size and absorbance evolution overtime (FIG. 2c and FIG. 15). As can be seen in FIG. 2c, the NP formation occurs within an initial fast growth phase, followed by a slower growth phase. The seeds formed in presence of 10% EtOH (44.5 nm) are smaller compared to 35% EtOH (62.9 nm) and the inflection point is shifted from 3 to 7 h by increasing the EtOH content. Additionally, absorbance measurements were conducted over time, since the polymerization of polydopamine is characterized by absorbance increase in the visible range (400-700 nm) (FIG. 14) indicated by a colour change from colourless to dark brown.^{[41,60,61]} The evolution of absorbance intensity also suggests slower polymerization with higher EtOH ratio. The impact of EtOH on the nucleation and growth kinetics of F127@PDA is in line with reported data on PDA nanoparticle formation.^{[62,63]} These findings were used to prepare F127@PDA nanocarriers of same relative composition but with different sizes (40, 60 and 100 nm) through co-polymerisation of DA and F127DA in a 20:1 molar ratio with 10%, 20% and 35% ethanol resulting in F127@PDA_40, F127@PDA_60 and F127@PDA_100, respectively.

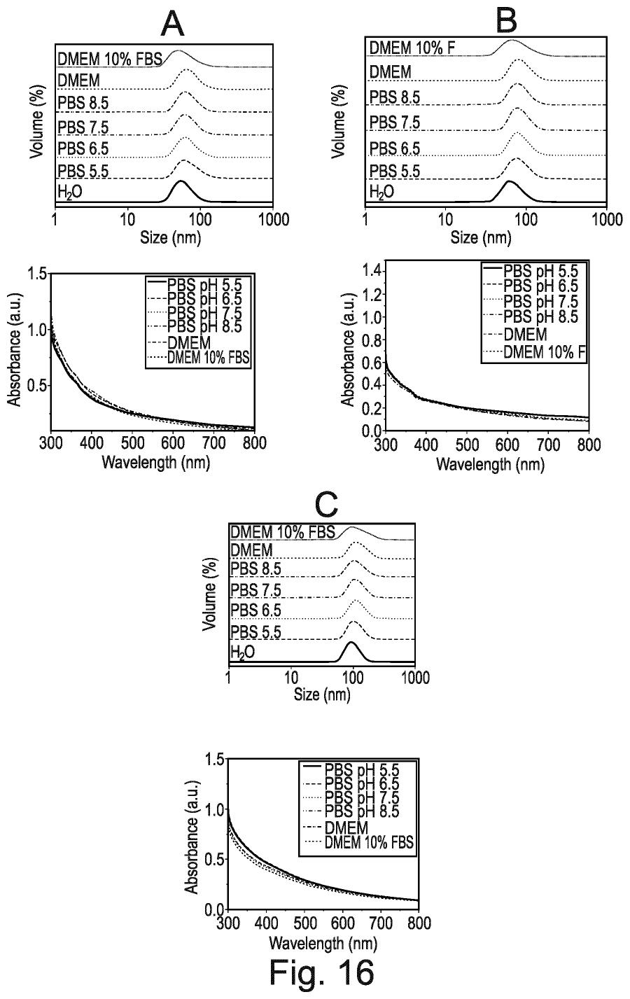

Prior to conducting cell uptake studies, the stability of the nanocarriers as well as the formation of protein corona was studied, since this can change the physicochemical properties of F127@PDA and thereby altering cell uptake.^{[64,65]} Dynamic light scattering and UV/Vis spectroscopy were used to assess colloidal stability of F127@PDA_40, F127@PDA_60 and F127@PDA_100 in physiological conditions (phosphate-buffer saline, PBS pH 5.5-8.5) and culture media containing serum proteins (DMEM with 0-10% FBS) (FIG. 16 and Table S2, SI). No significant changes in size and absorbance spectra were observed after 72 h incubation at 37° C. indicating high stability and that Pluronic F127 can shield the carrier surface from the formation of protein corona.

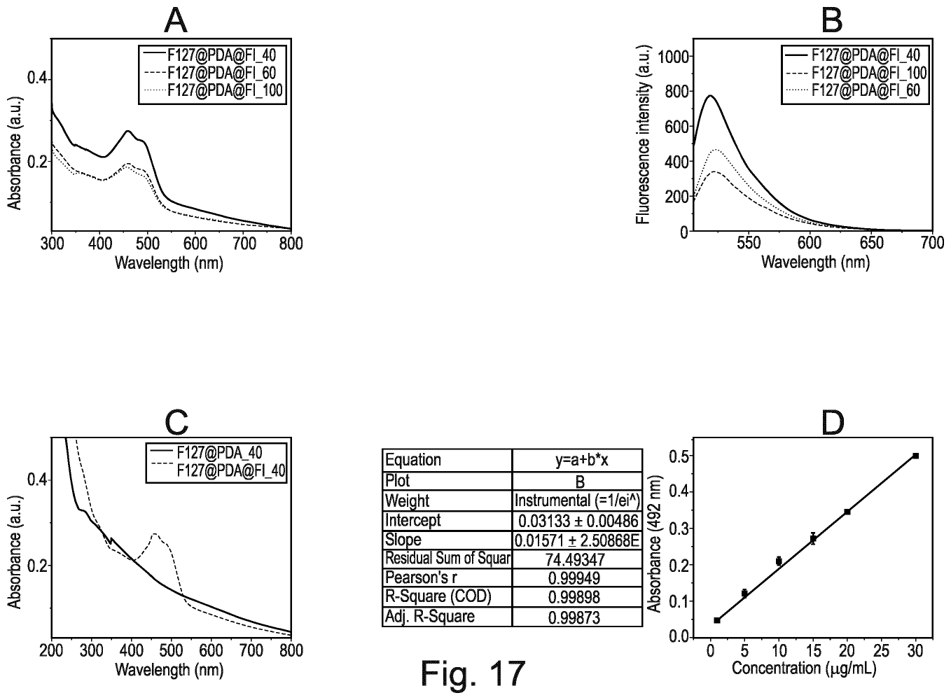

After successful synthesis and characterisation, fluorescent labelling was conducted, to enable nanocarrier tracking and to demonstrate the efficacy for post-synthetic modification of F127@PDA NPs. Amino-functionalized fluorescein (Scheme S2, SI) was attached via Michael addition due to the presence of quinones and indoles in the polydopamine backbone. The successful modification was validated through detection of the characteristic fluorescein peak in the absorbance and fluorescence spectra (FIG. 17, SI). The loading content of the fluorescein derivative was determined by UV/Vis and was found to be 9% for F127@PDA@_40 NPs and 6% for F127@PDA@FI_60 and F127@PDA@FI_100. No significant changes in size and zeta-potential were observed after the modification compared to F127@PDA NPs (Table S3, SI). It should be noted that this post-modification strategy can be employed to attach various (bio)molecular species containing thiol or amino groups as it was demonstrated by immobilisation of peptides^{[66]} and antibodies^{[67]} to polydopamine.

### Cytotoxicity Evaluation of F127@PDA NPs

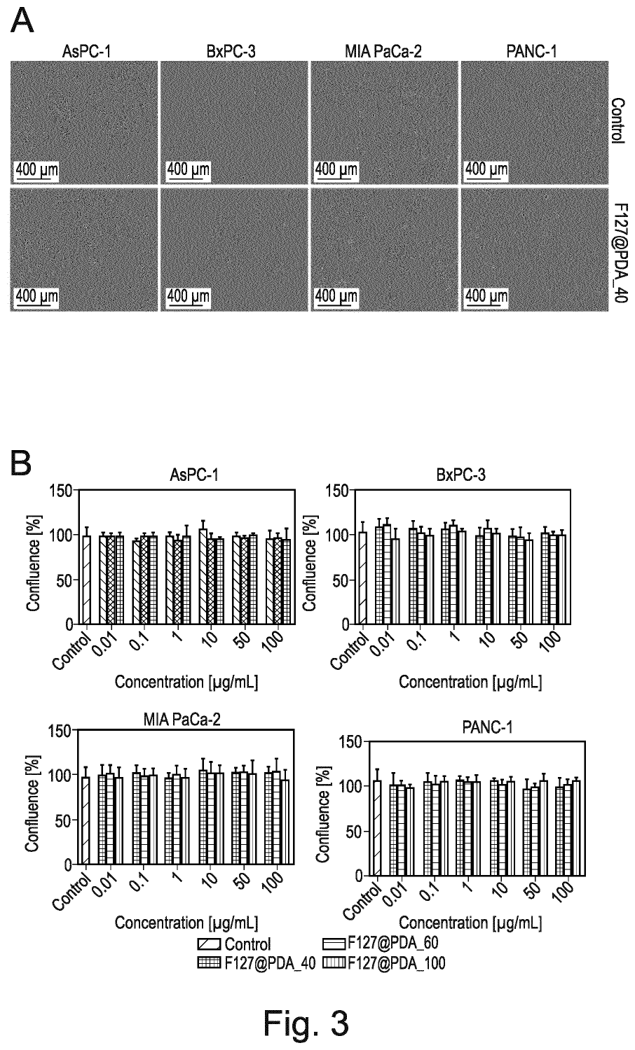

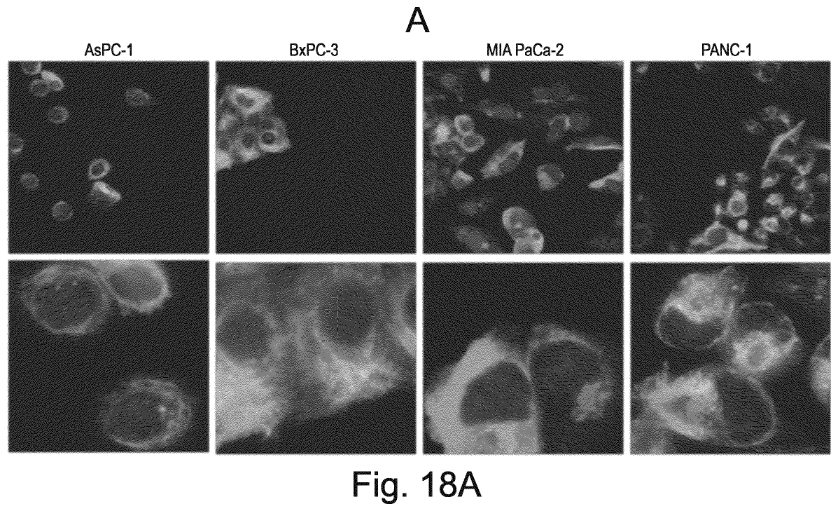

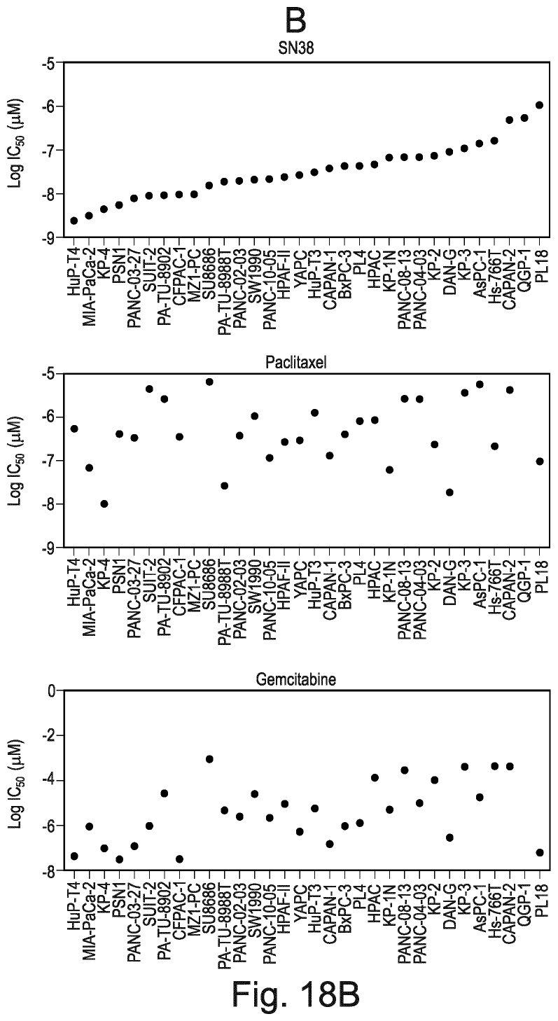

Pancreatic cancer is characterised with high morphological heterogeneity, which leads to poor drug-response.^{[68-70]} This heterogeneity needs to be taken into account during the study of cell uptake and toxicity to design efficient drug delivery systems (FIG. 18). For instance, more than 95% of pancreatic cancers carry KRAS^{G12} mutations and 70% p53 mutations.^{[71]} Therefore, in vitro cytotoxicity of F127@PDA_40, F127@PDA_60 and F127@PDA_100 has been assessed using four morphologically and genetically different pancreatic cancer cell lines, AsPC-1 (KRAS^{G12D}, Tp53^{C135s*35}), BxPC-3(wt-KRAS, Tp53^{Y220C}) MIA PaCa-2(KRAS^{G12c}, Tp53^{R248W}) and PANC-1 (KRAS^{G12D}, Tp53^{R273H}) over a range of concentrations (0.01-100 μg/ml). Cell growth of different cell lines treated with F127@PDA NPs was monitored by real time in vitro micro-imaging using the IncuCyte System (Sartorius, Germany) and MTS endpoint assay for 72 h. The IncuCyte system allows real time-point monitoring of cell growth by determining the confluence of the cells and displaying the morphological changes associated with the treatment. As shown in FIG. 3, none of the cell lines showed changes in morphology or density even when incubated with the highest concentration (100 μg/mL) of NPs over 72 h.

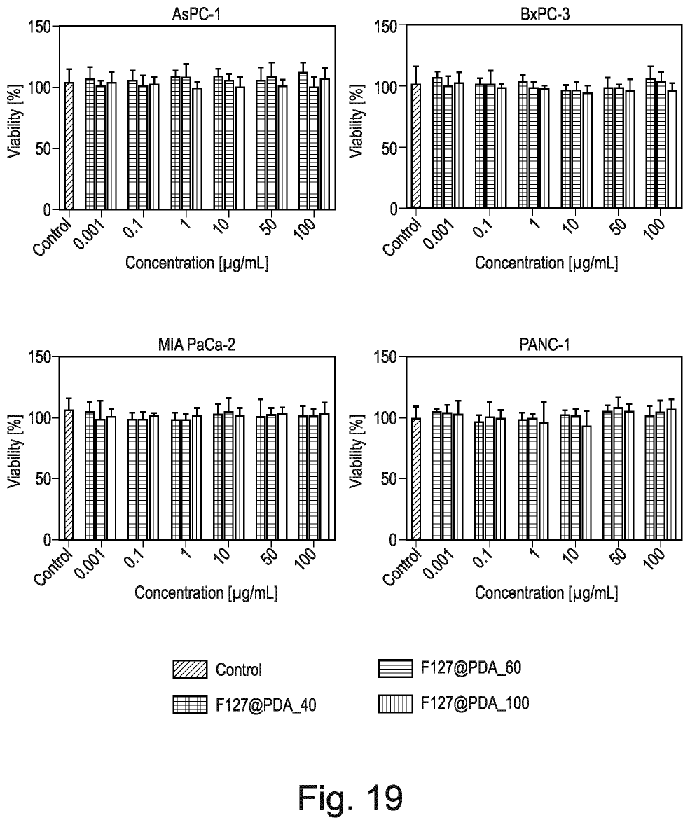

Live-cell analysis allows for quantification of the confluence percentage as a function of time, a value which is directly linked to the density of cells. The main advantage of live-imaging systems, compared to the common endpoint assays, such as MTS, nuclei count or CellTiter glow, is that it enables comparison between different time points and normalization of the data obtained in the same well over time.^{[74]} In addition, the built-in software quantifies the cell surface area coverage as confluency values, so that it is possible to express the cell growth as a ratio between end point and time zero, eliminating possible errors in cell seeding and interactions of the NPs with the colorimetric reagent. The analysis confirms that the F127@PDA particles show no significant difference compared to the control cells. Additionally, viability was also evaluated using widely employed MTS proliferation assay, confirming the results obtained by live cell imaging showing that the nanocarriers have no significant impact on the viability of the cells over the studied concentration range for 72 h (FIG. 19).

### Cell Uptake in Different Cell Types

The cell internalization of nanocarriers depends on their interactions with the cell membrane, which is generally followed by endocytosis.^{[72]} Various factors, such as the physicochemical and mechanical properties of the nanocarriers, as well as differences in cellular properties such as metabolic status, membrane protein expression and active trafficking pathways, influence the cellular uptake.^{[73,74]} To understand the cell uptake of our nanocarrier systems, flow cytometry and confocal studies were performed using fluorescein-labelled F127@PDA@FI_40, F127@PDA@FI_60 and F127@PDA@FI_100 nanocarriers in the four pancreatic cancer cell lines (AsPC-1, BxPC-3, MIA PaCa-2 and PANC-1). For both the internalization and intracellular localization studies, NPs were administrated for 24 h to minimize the proliferation effect that might result in dilution of intracellular NPs, since the doubling time of most mammalian cells is longer than 24 h.^{[75]}

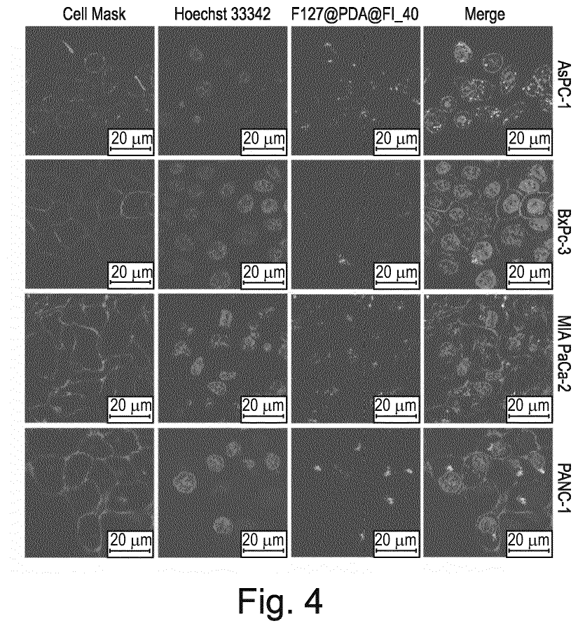

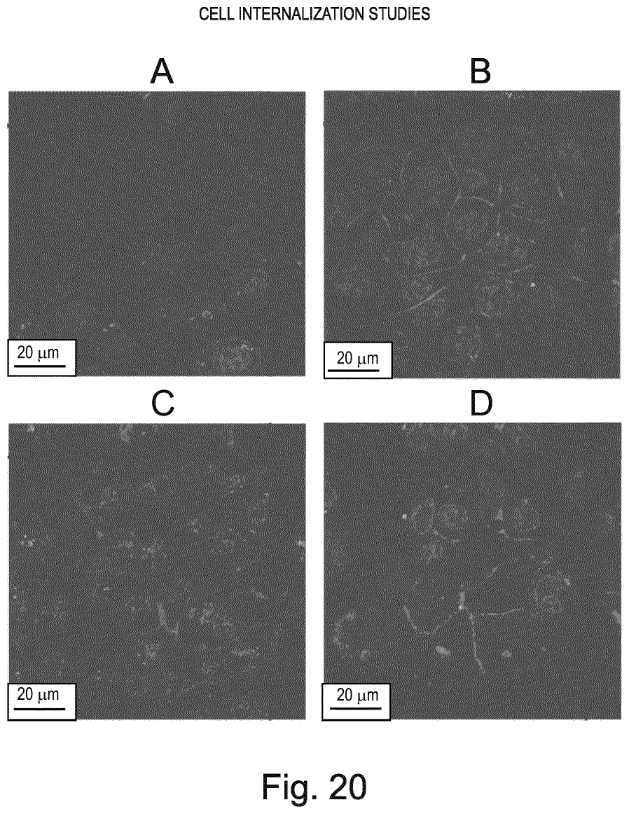

Cell membranes were labelled to differentiate intracellular NPs from those adhered to the cell surface. As shown by confocal microscopy images (FIG. 4), the NPs accumulate in the perinuclear region rather than being scattered throughout the cytoplasm. 3D images (z-stack) of the cells provide additional confirmation that the particles are internalized within the cells (FIG. 20, SI). Additionally, we found that a large number of NPs are accumulated on the surface of PANC-1, which was not observed for the other cell lines.

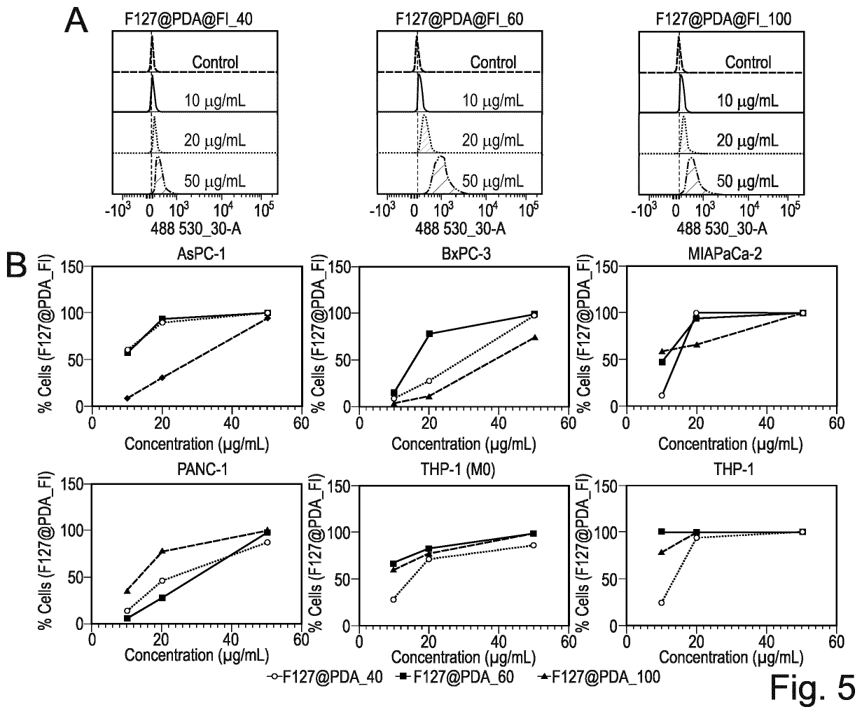

The impact of nanocarrier size on the uptake to different PDAC cells was further quantified using a well-defined flow cytometry method previously reported by Shin et al.^{[75]} First, the influence on the side scattering of the cells was measured with increasing F127@PDA@FI concentration as shown for BxPC-3 cells in FIG. 5a. No difference in the side scattering were observed after NP treatment for all cell selected lines (data not shown). Following this initial assessment, the fluorescein channel was used for quantification, and it was observed that the fluorescence distributions of the fluorescein channel shifts to higher intensities with increasing concentrations of NPs (FIG. 5a), consistent with a higher number of NPs in the cells.

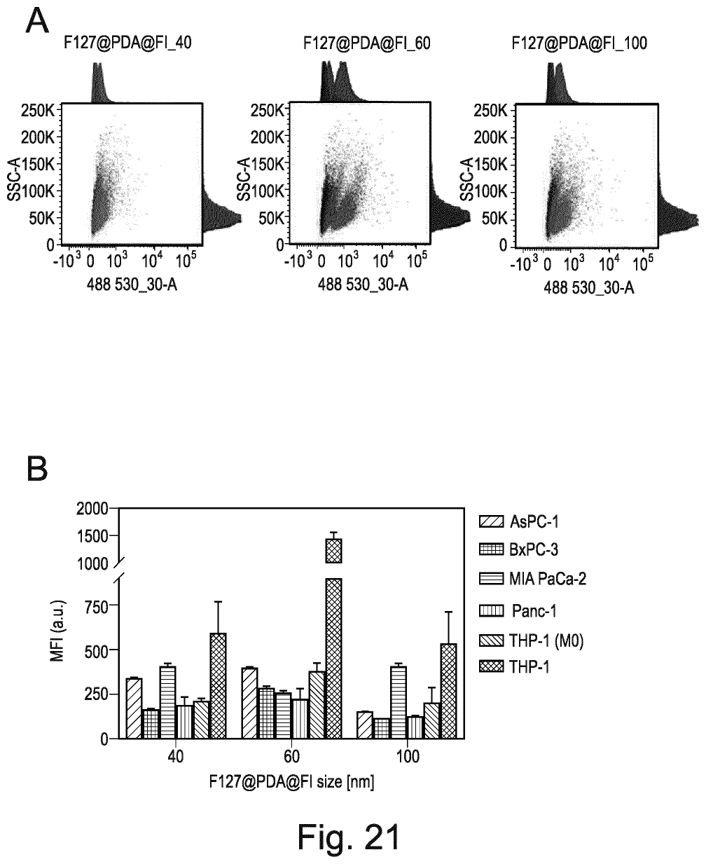

In addition to the different PDAC cell lines, the NP uptake within monocytic-like THP-1 cells and THP-1 differentiated macrophages (MO) was determined, as they were employed to assess the immunocompatibilty of the drug delivery system. The uptake of F127@PDA@FI increases in a dose-dependent manner within all PDAC cell lines (FIG. 21). Twenty μg/mL of the smaller (40 and 60 nm) NPs was sufficient to achieve cell uptake within more than 90% of the AsPC-1 and MIA PaCa-2 cells, while concentrations above 50 μg/mL were required to achieve the same uptake percentage within BxPC-3 and PANC-1 cells. For larger NPs (100 nm) 90% target loading in all PDAC cells was achieved above 50 μg/mL except AsPC-1 for which 20 μg/mL were sufficient (FIG. 5b). However, a significantly higher uptake level was noted in THP-1 cells, where the uptake was almost complete at lower concentrations (10 μg/mL), apart from the 40 nm particles. Similar results were previously reported for the cell uptake of TiO₂^{[75]} and polystyrene^{[76]} NPs, concluding that THP-1 cells take up nanoparticles much faster using a different uptake mechanisms. It should be noted that unlike other studied cells which are adherent, monocytic THP-1 cells are the only suspension cell line used to study F127@PDA NP uptake.

Additionally, the median fluorescence intensity (MFI) for individual cells was determined and normalized to the background level of each cell line.^{[77]} The MFI of suspension THP-1 cells is significantly higher compared to THP-1 (MO) and PDAC cells, which are adherent, and shows a greater variability compared to the other cell lines. Taking all of the data into account and comparing the uptake to THP-1(MO), F127@PDA_40 showed a higher level of overall uptake by PDAC cells compared to THP-1 (MO) and was therefore used for subsequent drug release studies.

Immunocompatibility and Interactions with Monocytes and Differentiated Macrophages

After intravenous administration, nanocarriers interact with different blood components, and interactions with the immune system as well as the clearance by the reticuloendothelial system are thought to be the main reason for the observed low levels of NPs at the tumour site.^{[78,79]} In addition, the modulation of the immune system can cause mild adverse reactions but also fatal immune complications. Although the evaluation of cytotoxicity caused by nanomaterials has become a standardized assay, the assessment of immunocompatibility is often disregarded at early stages of development.^{[21]} Nonetheless, in vitro evaluation of interactions with the immune system are relevant to determine the dose-range for in vivo studies and assess the safety and tolerance of the carrier.

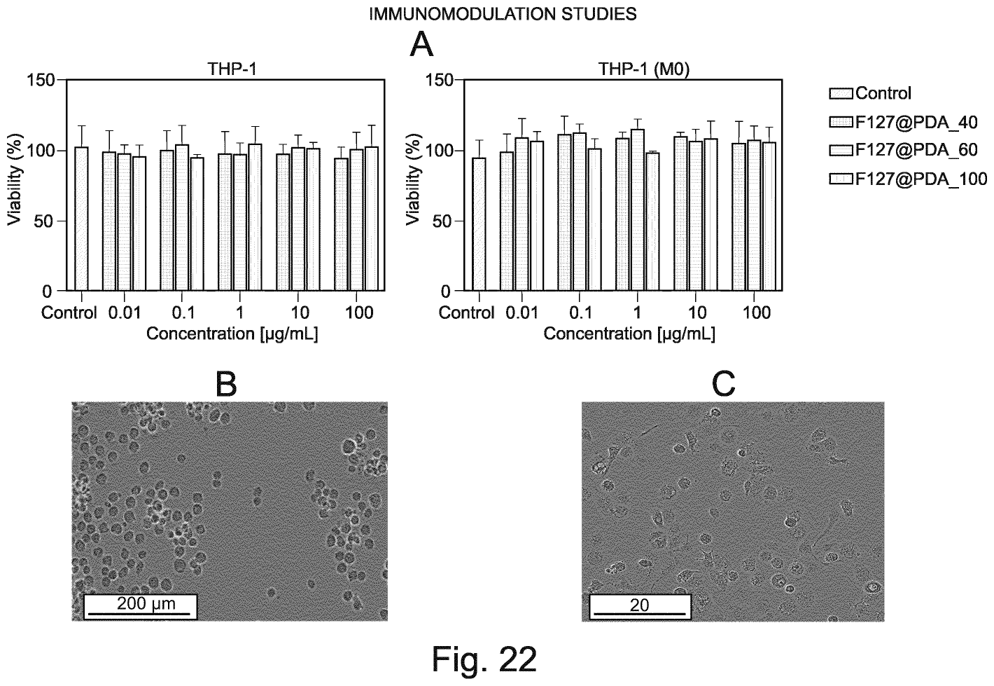

Monocytic THP-1 and differentiated THP-1 (MO) macrophages as cellular models to evaluate immunocompatibility.^{[80]} As outlined by Mottas et al.^{[22]}, when evaluating the interactions with monocytes and macrophages we are assessing whether our carriers are taken up by the cells, cause cell death and induce inflammatory response. Cell uptake studies showed that both THP-1 and THP-1 (MO) take up F127@PDA NPs, while cell toxicity studies demonstrated no significant effect on the viability of those cell lines after NP uptake (FIG. 22). Since monocytic THP-1 cells are suspension cells and post differentiation to THP-1 (MO) cells no longer proliferate, viability was not determined via live-cell imaging.

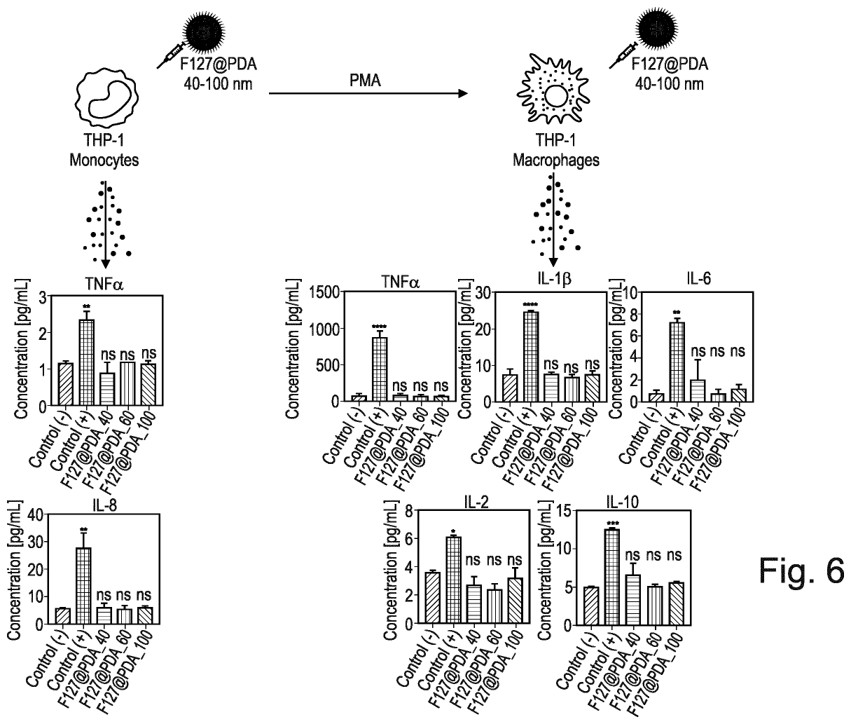

After cell uptake and viability assessment, cytokine profiling was conducted to determine whether F127@PDA NPs induce inflammation. Cytokines are proteins released by immune cells and are accepted as markers for the evaluation of immunotoxicity immunotoxicity or pro-inflammatory status^{[24]} The concentrations of proinflammatory (IL-1p, IL-2, IL-6, IL-8, TNF-α, IFN-γ) and anti-inflammatory (IL-4, IL-10, IL-12p70, IL-13) cytokines were determined to evaluate the immune effect of F127@PDA NPs (FIG. 6). The endotoxin lipopolysaccharide (LPS) was used as a positive control to assess the immune response as it significantly increases expression of TNF-α and IL-8 in THP-1 cells and IL-1p, IL-2, IL-6, IL-10 and TNF-α expression by THP-1 (MO) cells.^{[81]} After incubation with F127@PDA nanocarriers of different sizes (40, 60 and 100 nm), no statistical differences were observed in the cytokine expression compared to the control cells, confirming the immunocompatible of the F127@PDA nanocarriers towards both monocytes and macrophages. Further in vivo studies will be carried out to fully elucidate the biodistribution and immunocompatibilty of nanocarriers.

### Drug Loading and Release

Finally, before considering this carrier system for drug delivery, the drug loading efficiency and release profile in vitro was assessed. Current treatment options for PDAC include gemcitabine or a combination of gemcitabine with nab-paclitaxel (Abraxane) and, for fitter and younger patients, FOLFIRINOX (5-fluorouracil, leucovorin, irinotecan, and oxaliplatin), a combination of chemotherapeutics.^{[82]}

Pancreatic cancer cell lines AsPC-1, BxPC-3, MIA PaCa-2 and PANC-1 show different sensitivity towards the standard of care drugs (FIG. 18, SI) making them a suitable in vitro model to study the drug release profile of the chemotherapeutic drugs compared to the drug encapsulated within nanocarriers. SN38, the active metabolite of irinotecan (CPT-11), was used to evaluate drug delivery potential of F127@PDA nanocarriers. The metabolic conversion of CPT-11 to SN38 occurs in the liver through carboxylesterase 2 cleavage, however the expression can significantly vary from patient to patient.^{[83,84]} Additionally, both SN38 and CPT-11 can undergo lactone ring opening and form a less potent carboxylate form under physiological and basic conditions.^{[85]} Although SN38 is 1000-fold more potent than its prodrug,^{[86]} its clinical use is hindered due to the poor solubility and stability, making it an excellent candidate for nanocarrier delivery.^{[87-90]}

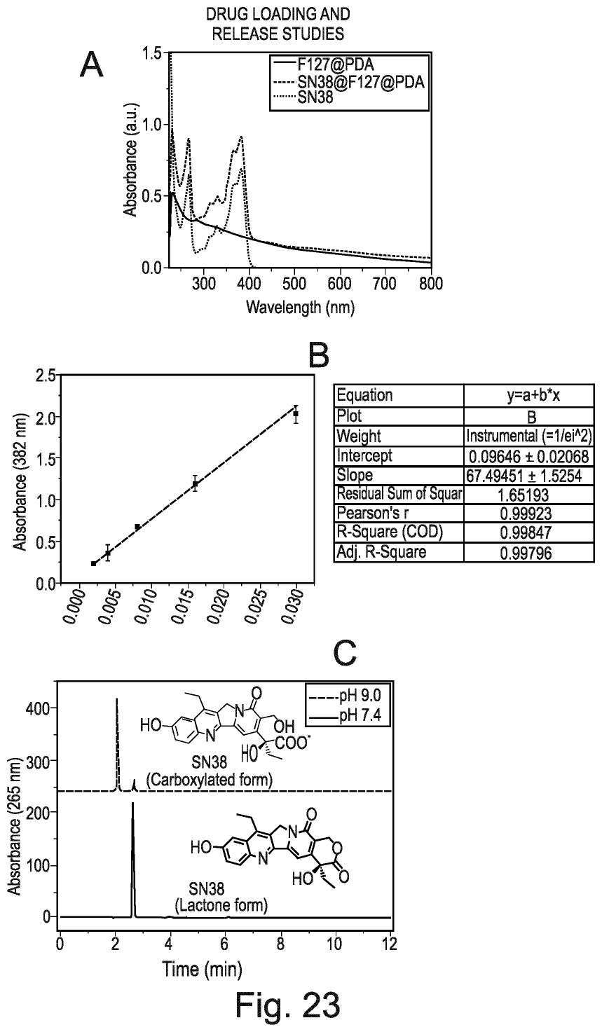

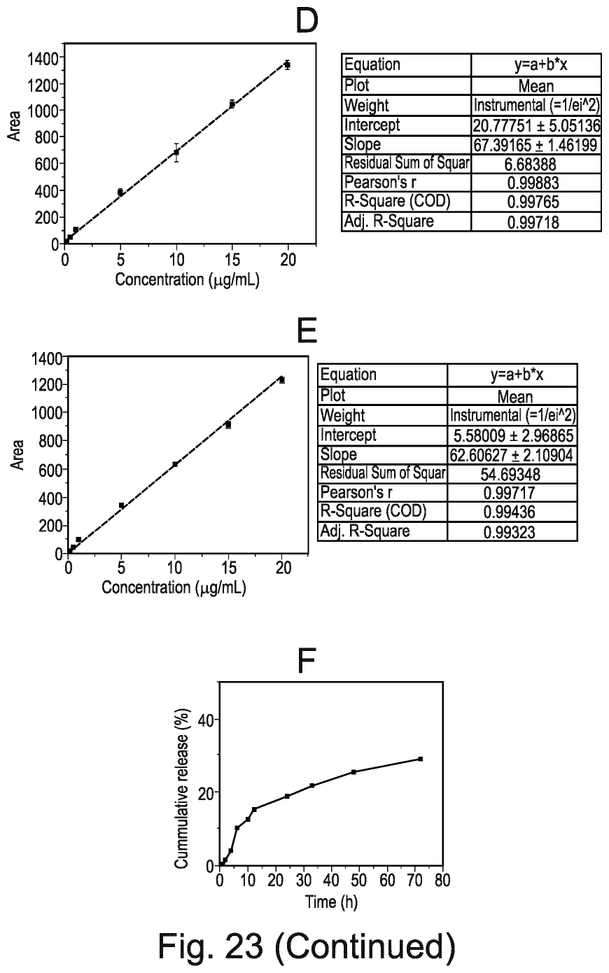

Based on cell uptake data, 40 nm F127@PDA were used to evaluate SN38 delivery. Due to the presence of aromatic groups in the polydopamine structure, small molecules can easily be absorbed through π-π stacking or hydrogen bonding.^{[41,91]} By mixing a suspension of SN38 and F127@PDA_40 NPs, a loading content of 13.1±3. 5% (w/w) was obtained, as verified by UV-Vis spectroscopy (FIG. 23, SI). To determine whether the lactone or carboxylesterase form of SN38 was loaded into the carrier, the supernatant was analysed using HPLC^{[92]} and showed the characteristic peak for the lactone at 2.7 min, indicating the loading of the active SN38 form. Prior to evaluating the in vitro effect of SN38@F127@PDA_40 on PDAC cell lines, release behaviour in PBS (1×, pH 7.4) was assessed (FIG. 23, SI) showing no burst release and overall release ˜30% after 72 h. However, it should be noted that the poor solubility of SN38 limits precise assessment and quantification of the release profile in physiological conditions.

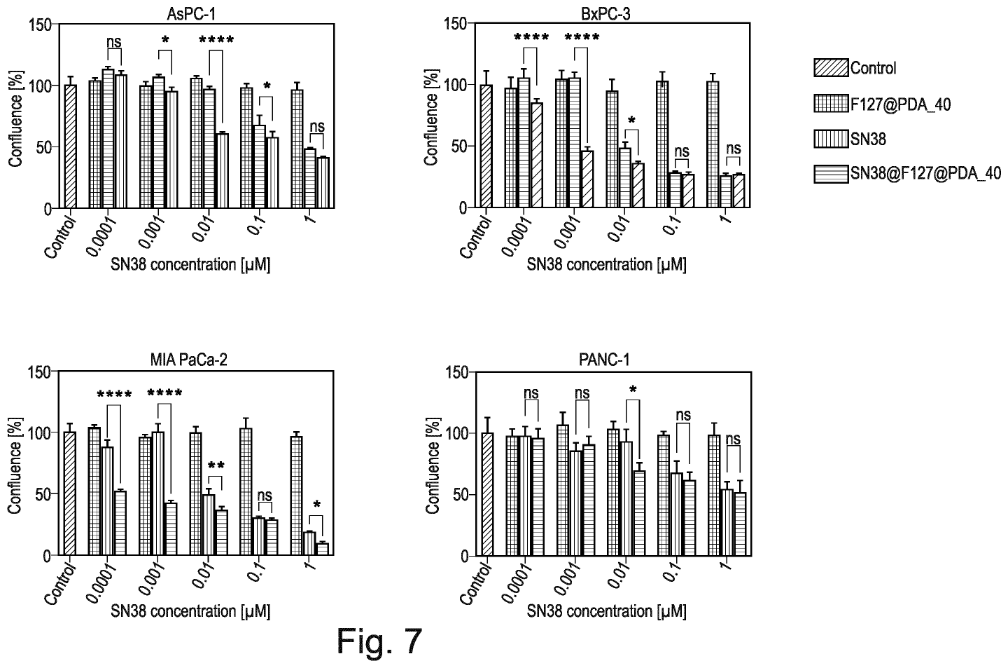

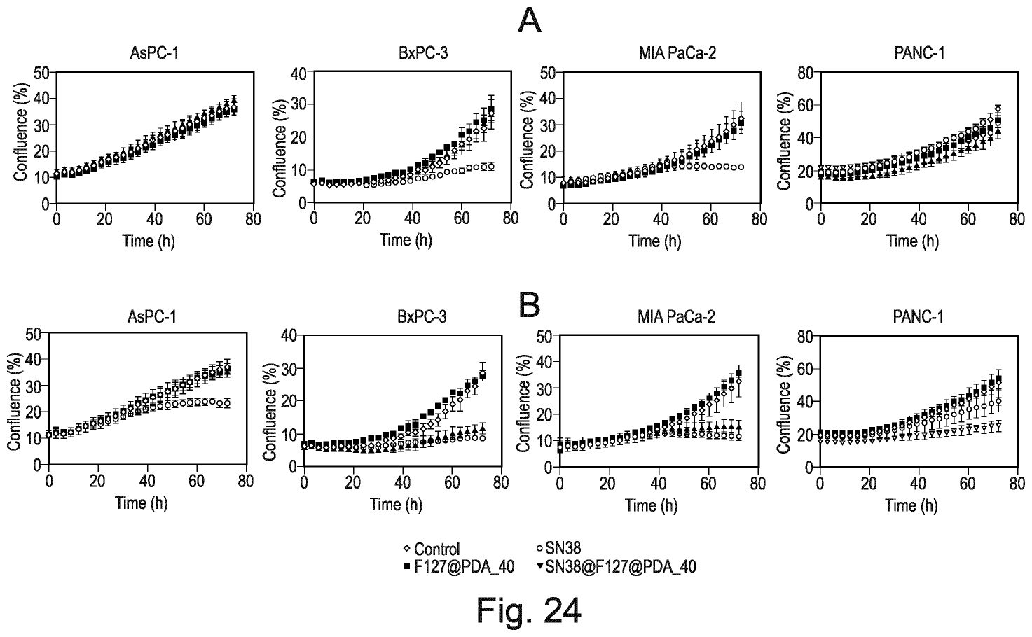

Next, the antiproliferative effect of SN38@F127@PDA_40 was assessed and compared to that of the free SN38 using live cell imaging. AsPC-1, BxPC-3, MIA PaCa-2 and PANC-1 cells were treated with SN38 and the same SN38 concentration in SN38@F127@PDA_40 for 72 hours. As shown in FIG. 7 and FIG. 24 at higher concentrations (1000 and 100 nM) SN38 loaded F127@PDA_40 NPs have a similar effect as free SN38. However, at concentrations lower than their IC₅₀ values (10 nM for AsPC-1 and PANC-1; 1 nM for BxPC-3 and MIA PaCa-2), a significant antiproliferative effect of the SN38 loaded F127@PDA_40 was observed considerably altering the growth curves over time (FIG. 24, SI). It should be noted that AsPC-1 and PANC-1 are both SN38-resistant cell lines. Yet, there is a more significant effect of SN38@F127@PDA on AsPC-1 cells than on PANC-1 cells, which can be explained by the higher cellular uptake of F127@PDA_40 (FIG. 5). Comparing individual cells lines, it was observed that PANC-1 cells show the least responsiveness to SN38 loaded nanocarriers. To improve uptake into PANC-1 cells future studies incorporating different Pluronic-containing polymers and targeting moieties will be conducted.

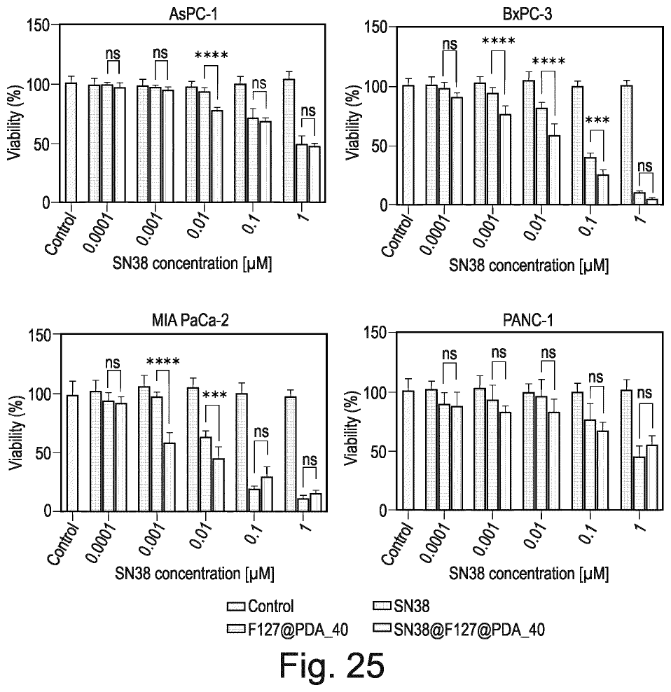

In addition to the proliferation studies, MTS assay was conducted (FIG. 25, SI) after treatment with SN38 and SN38@F127@PDA for 72 h and IC₅₀ values were calculated (Table S4, SI). The obtained IC₅₀ values for SN38 calculated from the MTS assay correspond to literature reports^{[93]} and SN38@F127@PDA NPs show lower IC₅₀ values then SN38 for all tested cell lines. The IC₅₀ values obtained from the live-cell imaging were lower than from MTS both for SN38 and SN38@F127@PDA. Similar findings were also noted for several other drugs using real-time cell analysis,^{[94-96]} which shows the importance to use different methods when determine the effect of new therapies to avoid generating misleading results.

### Conclusion

In summary, we have designed a novel size-adjustable and easily modifiable drug delivery system that contains Pluronic F127 functionalized polydopamine and assessed its potential for drug delivery of hard-to-treat solid tumour cancers such as pancreatic cancer. While PDA-based NPs systems have been investigated for drug delivery in several cancer types, to the best of our knowledge, this is the first PDA-based NP system evaluated for the treatment of solid tumour cancers. This study highlights several assays for in vitro assessment, which take into account the heterogeneity of PDAC, and can be used as a tool to gain better understanding of drug delivery systems based on the disease characteristics at an early stage. The F127@PDA carriers were prepared in different sizes without altering the composition, showed excellent colloidal stability and high loading capacity for hydrophobic and labile irinotecan prodrug SN38. Viability, cellular uptake and cytokine profiling studies of differently sized nanocarriers (40, 60 and 100 nm) demonstrated high bio- and immunocompatiblity for all studied sizes. However, cell uptake into the different PDAC cells, monocytes and macrophages showed variability based on NP size. Finally, SN38 loaded F127@PDA nanocarriers showed a more pronounced effect on proliferation of all cell lines compared to the free drug. The efficacy of the drug delivery system was in related to the cell-uptake data of the NPs showing a higher antiproliferative effect for AsPC-1 cells compared to PANC-1, which are SN38-resistant.

### Experimental Section

All reagents unless otherwise stated were purchased from Sigma Aldrich (UK), Acros Organics (UK) or TCl chemicals and used without further purification. ¹H NMR spectra were recorded on a 400 MHz DCH Cryoprobe Spectrometer in CDCl₃ and DMSO-d₆. UV/Vis absorption spectra were obtained on an Agilent Cary 300 Spectrophotometer. Fluorescence emission spectra were obtained using a Varian Cary Eclipse Fluorescence Spectrophotometer using excitation and emission splits of 5 nm. DLS and zeta potential measurements were recorded using a Zetasizer Nano ZS instrument (Malvern Panalytical, UK) with a sample concentration of 0.5 mg/mL. All Measurements were conducted three times with 15 subruns for each sample. Error bars represent the standard deviation of three measurements Zeta potential was measured in a folded capillary Zeta cell DTS1070 (Malvern, UK). FTIR spectroscopy was carried out using a Bruker Tensor 27 spectrometer with samples pressed into KBr pellets. Lyophilization was carried out using a Telstar LyoQuest benchtop freeze dryer (0.008 mBar, −70° C.).

### Synthesis and Characterisation of F127@PDA NPs

### General Procedure for the Synthesis of Pluronic-Polydopamine (F127@PDA)

Trizma-base (22.5 mg) was dissolved in 2.5 mL Milli Q water and added to a mixture of ethanol and MilliQ water (30 mL) and stirred for 30 min at room temperature. Dopamine hydrochloride (dissolved in 1 mL Milli Q water) and F127DA (dissolved in 1 mL ethanol) were mixed and sonicated before being added dropwise to the reaction mixture. The mixture was left to stir over night at room temperature resulting in a dark brown solution. The reaction mixture was washed with Milli-Q water using Vivaspin 20 (Satorius, UK) centrifugal concentrators (30 kDa MWCO) for 15 min at 4000 rpm until the supernatant was colourless. The obtained particles were diluted in Milli-Q water, frozen in liquid nitrogen and lyophilised to yield a dark brown powder.

For comparison, samples with varying molar ratio of DA:F127DA or varying volume percent of ethanol in the reaction mixture were prepared, while keeping the other synthesis parameters the same (Table S1, ESI).

To investigate the formation kinetics of F127@PDA NPs with varying ethanol percentage, the absorbance changes at 400 nm were measured with UV-Vis and hydrodynamic size using Zetasizer Nano. At each time point 50 μL of the reaction solution was taken and the absorbance was measured.

### Colloidal Stability of F127@PDA NPs

The colloidal stability of NPs, with and was evaluated in deionised water, PBS (1×, pH=5.5-8.5), phenol-red free DMEM and DMEM+10% FBS. PBS was adjusted to different pH by adding HCl or NaOH. Solutions containing 900 μL of solvent and 100 μL of 1.0 mg mL⁻¹ sample were incubated for 72 hours at 37° C. After 72 hours hydrodynamic size, zeta potential and UV-Vis were measured.

### General Procedure for Fluorescent Labelling of F127@PDA

F127@PDA NPs (5 mg) was dissolved in 10 mM Tris buffer (5 mL) and Fluorescein-TEG-NH₂ (10 mg, 2 wt eq.) dissolved in 0.5 mL ethanol was added dropwise. The mixture was protected from direct sunlight and stirred over night at room temperature. Excess Fluorescein-TEG-NH₂ was removed washing with Milli-Q water using Vivaspin 20 (Satorius, UK) centrifugal concentrators (10 kDa MWCO) for 15 min at 4000 rpm until the supernatant was colourless, followed by 3 days dialysis against water with 12-14 kDa MWCO dialysis bags.

### In Vitro Evaluation of F127@PDA NPs

### Cells Lines and Growth

Human pancreatic cancer cell lines AsPC-1, BxPC-3, MIA PaCA-2 and PANC-1 were purchased from American Type Culture Collection (ATCC). MIA PaCa-2 and PANC-1 were grown in DMEM (Sigma, UK) supplemented with 10% FBS. AsPC-1 and BxPC-3 cells were cultured using RPMI supplemented with 10% FBS. Human epithelial cell line ARPE-19 (CRL-2302) were bought from American Type Culture Collection (ATCC) and grown in DMEM:F12 Medium supplemented with 10% FBS. All the cell lines were cultured in a humidified environment at 37° C. with 5% CO₂. All cell lines were routinely tested to confirm the absence of Mycoplasma and verified by STR profile. In vitro experiments were conducted with 60% to 80% confluent cultures at passage number between 5 and 15.

### Live-Cell Cytotoxicity Studies

All NPs were tested for cytotoxicity studies in AsPC-1, BxPC-3, MIA-PaCa-2, PANC-1 and ARPE-19. Cells were seeded into 96-well plates at concentration of 2000 cells/well, in 200 μl of complete growth medium and incubated at 37 C, 5% CO₂ for 24 h. After overnight incubation, the cells were treated with different concentration of F127@PDA-40, F127@PDA-60 and F127@PDA-100 (0.01-100 μg/mL) dissolved in complete cell media, the same volume of water was added for the negative control. The plates were then inserted into the IncuCyte®S3 Live-Cell Analysis System (Sartorius) for real-time imaging. Treated plates were imaged every 3 h for 72 h under cell culture conditions with 10× objective using the brightfield channel. Mean cell confluence was calculated using the images taken from 3 random fields of view per well using the IncuCyte S3 v2017A software. All Incucyte experiments were performed in triplicate in three independent experiments. Relative confluence values were obtained by normalizing each value to the time zero value in each sample and normalised to the untreated control sample.

$$Confluence ( \% ) = 100 \times ( \frac{Confluence of treated cells 72 h / Confluence of treated cells 0 h}{Confluence of control 72 h / Confluence of control 0 h} ) \qquad ( 1 )$$

### MTS Cytotoxicity Studies

Additionally, the effect of F127@PDA NPs on the viability of AsPC-1, BxPC-3, MIA PaCa-2, PANC-1 and ARPE-19 using MTS assay (Promega, USA.) Cells were seeded into clear 96-well plates containing 2000 cells/well in 100 μL complete growth medium and cultured for 24 h at 37° C. and 5% CO₂. Subsequently, cells were treated with varying concentrations of F127@PDA_40, F127@PDA_60 and F127@PDA_100 (0.01-100 μg/mL) dissolved in complete growth media containing 0.1% water. After further 72 h incubation at 37° C. and 5% CO₂, 20 μL of CelTiter 96® AQᵤₑₒᵤₛ One Solution (Promega, USA) was added into each well and incubated at 37° C., 5% CO₂ for 1-4 h, according to the manufacturer's instruction. The absorbance of each well was measured at 490 nm using a Spark plate reader (TECAN, CH). Control measurements included negative control of cells with DMEM, cells with DMEM containing 0.1% water, cell-free culture media (blank) and cell-free sample dilutions in culture media to evaluate potential sample interferences with MTS reagent. All experiments were conducted in biological triplicates. The percentage cell viability was calculated according to the following:

$$Cell viability ( \% ) = 100 \times ( \frac{Absorbance of treated cells - Absorbance of blank}{Absorbance of control - Absorbance of blank} ) \qquad ( 2 )$$

### Immunocompatibility Evaluation of F127@PDA NPs

### Viability Test on Acute Monocyte Leukaemia Cell Line (THP-1)

THP-1 cells were kindly provided by Dr. Hassan Rahmoune (Department of Chemical Engineering and Biotechnology, University of Cambridge, UK) and maintained in RPMI 1640 medium with L-glutamine and sodium bicarbonate (Sigma), supplemented with 10% FBS (Gibco) and 1% penicillin-streptomycin (Thermo Fisher Scientific). THP-1 differentiation was induced by phorbol-12-myristate 13-acetate, 100 nM (PMA, Sigma-Aldrich) for 48 h. After differentiation the medium was replenished with full growth media and the cells were incubated for additional 24 hours at 37° C. and 5% CO₂. To determine the effect of F127@PDA NPs on the viability of THP-1 cells and THP-1 differentiated cells were seeded into a 96-well plate (3000 cells/well) and incubated with varying concentrations of F127@PDA_40, F127@PDA_60 and F127@PDA_100 (0.01-100 μg/mL) for 72 hours. Following incubation MTS assay was performed as described in the previous section.

### Cytokine Analysis in THP-1 and THP-1 M(0)

The cytokine analysis was conducted according to the procedure described by Zhu et al.^{[97]} Briefly, THP-1 and THP-1 M(0) cells (1×10⁵ cells/mL) were seeded in a 24-well plate and treated with 10 μg/mL F127@PDA_40, F127@PDA_60 and F127@PDA_100 for 24 h. Lipopolysaccharide (LPS, 10 ng/mL) was used as a positive control. After incubation 1 mL cell media form individual cells was centrifuged at 1000 rpm for 5 min and the supernatant was collected and kept at −80° C. for cytokine analysis. The quantification of multiple cytokines in the samples was conducted via Meso Scale Discovery (MSD) multiplex assay platform. The MSD assay is an ultrasensitive electrochemical luminescence immunoassay performed on the MesoScale Diagnostics Sector Imager 6000. The samples were analysed at the Core Biochemical Assay Laboratory (NHS Cambridge University Hospitals; UK).

### Immunofluorescence Staining of PDAC Cell Lines

Cells were seeded onto 96-well treated plates (Perkin Elmer) at 20% confluence. Cells were left for 24-72 h before incubation with cell mask deep red stain (ThermoFisher) and then imaging live or fixing with 4% paraformaldehyde (PFA) and washed three times in PBS. For fixed cell samples, cells were blocked in 2% w/v bovine serum albumin (BSA) in PBS for 30 min before incubation with primary antibodies for 1 h at room temperature. Cells were imaged using the Operetta spinning disk confocal microscope (Perkin Elmer) using the 63× water objective. Primary antibodies used for immunofluorescence were mouse anti-LAMP-1 (BD Bioscience), mouse anti-EEA-1 (BD Bioscience), Cis-Golgi (Abcam), mouse anti-alpha tubulin (DM1A, Cell Signalling) and rat anti-tubulin (Alexa Fluor®647, Abcam). Secondary antibodies used were donkey anti-rabbit (Alexa Fluor®488) and goat anti-mouse (Alexa Fluor®555) sourced from Abcam. Nuclei were stained with Hoechst (Thermo Fisher).

### Confocal Imaging

Cells were seeded into a glass bottom dish (MatTek Life Science, US) at concentration of 200 000 cells/ml and incubated at 37° C. for 24 h, then treated with different F127@PDA@FI NPs at different concentrations for 24 h at 37° C. After 3 washes with 1×PBS the cells were stained with CellMask™ Deep Red (Thermo Fischer) Plasma membrane stain and Hoechst 33342 (Thermo Fisher) according to the manufacturer's instructions. Cells were then washed gently with PBS for three times and imaged using confocal microscope (Axio Observer Z1 LSM 800, Zeiss). Zen software (Zeiss) was used for the acquisition image processing.

### Flow Cytometry Analysis

Cells were seeded in 6-well plates at a density of 2×10⁵ cells per well and cultured for 24 h. The next day cells were treated with various concentrations of F127@PDA@FI_40, F127@PDA@FI_60 and F127@PDA@FI_100 NP solutions prepared in the culture media (10 μg/mL, 20 μg/mL or 50 μg/mL) and incubated for 24 h. After the treatment, cells were washed three times with 1×PBS to remove residual NPs both in culture media and on the cell surfaces, they were detached with 0.25 mL TrypLE (Thermo Fischer, UK) and centrifuged for 5 min at 300 g. 1 mL of FACS buffer (PBS with 4% FBS) was added to the cells and 10 μL of 10 μg/mL DAPI stock solution. The cells were kept at 4° C. until flow cytometry analysis. Flow cytometry was carried out on a Canto II flow cytometer (BD Biosciences) using 355 and 488 lasers. 10000 events were acquired for each sample. FlowJo software (version 10.2) was used for data analysis. Briefly, the live single-cell population was gated in a plot of FSC vs. SSC after excluding cell debris and doublets a histogram from the FITC channel for the single-cell population was obtained and analysed.

### Drug Loading and Release Studies

To assess the loading capacity, absorption of SN38 was investigated adapting a method by Wang et al.^{[90]} A suspension of 10 mg F127@PDA NPs (1 mg/mL) and 2.5 mg SN38 (0.25 wt eq.) in DMSO:H₂O=1:10 (10 mL) were sonicated for 30 min, followed by stirring at room temperature for 72 hours. To remove free drug, the reaction mixture was first centrifuged at 2000 rpm for 5 min. The supernatant was collected and washed with Milli-Q water using Vivaspin 20 (Satorius, UK) centrifugal concentrators (10 kDa MWCO) for 15 min at 4000 rpm. The obtained particles were diluted in Milli-Q water, frozen in liquid nitrogen and lyophilised to yield a dark brown powder. The loading content of SN38 within the NPs was determined using UV-Vis. The absorbance of SN38@F127@PDA NPs at 382 nm was deducted by the F127@PDA absorbance and the loading content was calculated according to the following equation: (3).^{[98]}

$$Loading content ( weight [ wt ] \% ) = \frac{Mass of SN 38 in nanocarrier}{Initial mass of nanocarrier} \times 100 \qquad ( 3 )$$

Next, drug release was measured in PBS (1×, pH 7.4). 2 mL of 1 mg/mL SN38@F127@PDA NPs were dissolved in PBS (1×, pH 7.4) and placed in a dialysis bag (MWCO 12-14 kDa) and dialyzed against PBS (25 mL) in an incubator shaker at 37 C. 1 mL of the media was removed and replaced with fresh media at different time intervals. The amount of the released drug was quantified using HPLC as described by Xuan et al.^{[92]} Briefly, the HPLC analysis was conducted on an an Agilent 1260 Infinity Quaternary LC equipped using an Agilent Zorbax SB-C18 (4.6 mm×250 mm, 5 μm) analytical column. The mobile phase consisted of a mixture of NaH₂PO₄ (pH 3.1, 25 mM) and acetonitrile (50:50, v/v) with a 1 mL/min flow rate. SN38 concentration was detected at 265 nm and an external calibration curve for both SN38 forms (carboxylic acid and lactone) were used for quantification.

### Drug Release In Vitro

Cells were seeded into 96-well plates at concentration of 2000 cells/well, in 100 μl of complete growth medium and incubated at 37 C, 5% CO₂ for 24 h. After overnight incubation, the cells were treated with SN38 and the same concentration of SN38@F127@PDA using various concentrations (0.0001 μM-1 μM). The plates were inserted into the IncuCyte®S3 Live-Cell Analysis System (Sartorius) for real-time imaging. Treated plates were imaged every 3 h for 72 h under cell culture conditions with 10× objective using the brightfield channel. Average cell confluence was calculated as described in the previous section. In addition to live cell imaging after 72 h incubation in the IncuCyte®S3 MTS assay was performed as described in the section above.

### Statistical Analysis

Experiments were independently repeated at least in triplicates unless otherwise noted and all data presented as mean±standard deviation. All statistical analysis was done with Graphpad Prism 9 software (GraphPad Software, San Diego, CA, USA). Significance levels are defined as the following: ns for p>0.05, * for p≤0.05, ** for p≤0.01, *** for p<0.001, and **** for p<0.0001.

### Supporting Information

### Materials and Methods

All materials were purchased from either Acros Organics (UK), Alfa Aeser (UK), Sigma-Aldrich (UK) or TCl Chemicals (UK) in the highest purity available and used without further purification.

### Characterisation Techniques

¹H measurements were carried out using 400 MHz QNP Cryoprobe Spectrometer (Bruker) by the NMR service of the Department of Chemistry, University of Cambridge. UV-Vis absorption spectra were obtained with an Agilent Cary 300 Spectrophotometer. Fluorescence emission spectra were obtained using a Varian Cary Eclipse Fluorescence Spectrophotometer using excitation and emission splits of 5 nm. DLS and zeta potential measurements were recorded using a Zetasizer Nano Range instrument (Malvern Panalytical). FTIR spectroscopy was carried out using a Bruker Tensor 27 spectrometer with samples pressed into KBr pellets. SEM images were obtained using a FEI Verios 460. Samples were suspended in water and drop cast on lacey carbon copper grids (Agar Scientific).

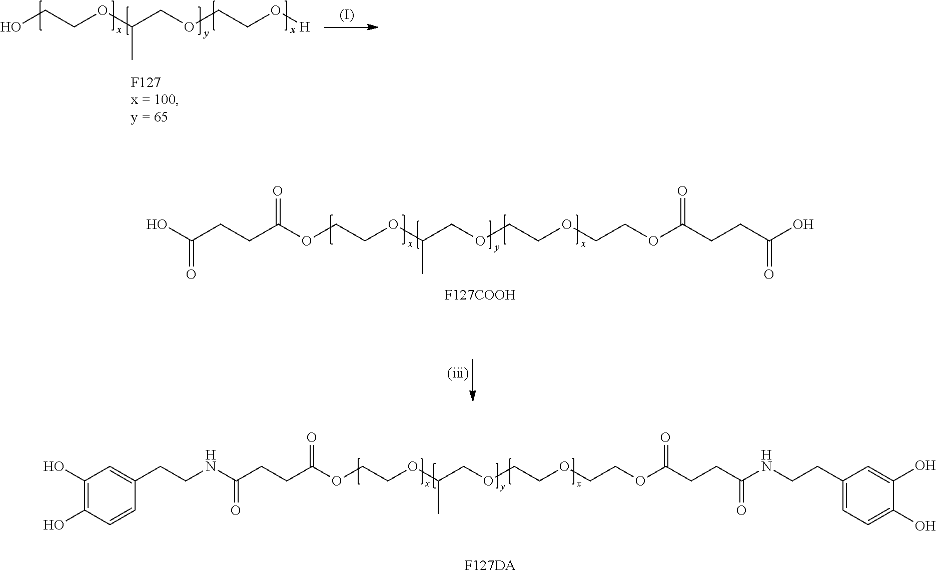

### Synthesis of Pluronic-Dopamine Monomer (F127DA)

### Carboxyl-Terminated Pluronic (F127COOH)

Carboxyl-terminated F127 (F127COOH) was prepared according to the procedure reported by Li et al.^{[56]} F127 (30.0 g, 2.5 mmol) was dissolved in pyridine (60 mL) and succinic anhydride (7.1 g, 71.4 mmol) was added. The reaction mixture was stirred under argon for 72 hours. Subsequently, CH₂Cl₂ (150.0 mL) was added to dilute the reaction mixture and washed with saturated sodium chloride solution three times. The organic layer was dried over anhydrous magnesium sulphate overnight, filtered, and concentrated by rotary evaporation. The residue was precipitated with cold diethyl-ether (31.5 g, yield: 95%). ¹H NMR (400 MHz, CDCl₃): δ (ppm) 1.42-0.84 (m, 195H, CH₃-a), 2.66-2.51 (m, 8H, CH₂-f,g), 3.45-3.26 (m, 67H, CH-b), 3.55-3.42 (m, 132H, CH₂-c), 3.85-3.54 (m, 833H, CH₂-d), 4.29-4.18 (m, 4H, CH₂-e).

### Pluronic-Dopamine (F127DA)

F127COOH (2.0 g, 0.2 mmol) was dissolved in DMF (25 mL) followed by addition of NHS (60.2 mg, 0.52 mmol), DMAP (2.5 mg, 0.02 mmol), DCC (120.5 mg, 0.58 mmol) and dopamine hydrochloride (65.5 mg, 0.45 mmol). The reaction mixture was stirred under inert atmosphere for 24 hours. The solvent was removed by rotary evaporation and the resulting product was subsequently dissolved in methanol:water (50:50), dialyzed against methanol:water (50:50) for 2 days, and then against water for another 2 days. The final product was obtained in the form of a white power after lyophilization of the dialyzed solution (1.95 g, yield: 78%). ¹H NMR (400 MHz, CDCl₃): δ (ppm) 1.40-0.89 (m, 195H, CH₃-a), 2.44-2.31 (m, 4H, CH₂-e), 2.71-2.52 (m, 12H, CH₂-f,g,i), 3.41-3.29 (m, 67H, CH-b), 3.55-3.42 (m, 132H, CH₂-c), 3.81-3.55 (m, 843H, CH₂-d), 4.22-4.12 (m, 8H, CH₂-e,h), 6.11-5.96 (m, 2H, NH), 6.51 (dd, J=8.0, 1.5, 2H, Ar—H), 6.67 (d, J=1.2, 2H, Ar—H), 6.76 (d, J=8.0, 2H, Ar—H), 8.09-8.02 (m, 4H, OH).

### Synthesis and Characterisation of F127@PDA NPs

TABLE S1
DLS and zeta potential (ζ) measurements of F127@PDA samples.
Errors are the standard deviation of the triplicate data. Sizes from
STEM images were determined from the mean of >100 measurements of
spherical particles, with the associated error being the standard deviation.

| | | | | | Hydrody- | | STEM |
|---|---|---|---|---|---|---|---|
| n(DA): | m(DA) | m(F127DA) | V(EtOH) | V(H₂O | namic size | | size |
| n(F127DA) | (mg) | (mg) | (mL) | (mL) | (nm) | PDI | (nm) |
| 20:1 | 13.7 | 45.7 | 3 | 27 | 59.2 ± 1.3 | 0.060 | 45.9 ± 5.4 |
| 20:1 | 13.7 | 45.7 | 6 | 24 | 76.6 ± 1.2 | 0.048 | 62.5 ± 5.9 |
| 20:1 | 13.7 | 45.7 | 9 | 21 | 104.4 ± 0.8 | 0.005 | 87.8 ± 9.0 |
| 20:1 | 13.7 | 45.7 | 10.5 | 19.5 | 117.0 ± 1.7 | 0.047 | 103.3 ± 14.9 |
| 20:1 | 13.7 | 45.7 | 15 | 15 | 164.1 ± 4.6 | 0.021 | 140.9 ± 15.4 |
| 10:1 | 13.1 | 91.6 | 6 | 24 | 95.8 ± 2.2 | 0.027 | 73.4 ± 7.2 |
| 20:1 | 13.7 | 45.7 | 6 | 24 | 106.5 ± 1.4 | 0.032 | 87 ± 7.8 |
| 50:1 | 13.9 | 32.5 | 6 | 24 | 120.4 ± 3.5 | 0.043 | 99.3 ± 9.7 |
| 100:1 | 14.0 | 24.5 | 6 | 24 | 141.8 ± 4.2 | 0.009 | 110.2 ± 9.9 |

### Colloidal Stability of F127@PDA NPs

TABLE S2
Hydrodynamic size and zeta potential (ζ) for F127@PDA_40, F127@
PDA_60 and F127@PDA_100 in water, PBS pH 5.5-8.5 and DMEM with FBS
0-10% after 72 hours incubation at 37° C. Errors are standard deviations of the triplicate data.

| | F127@PDA_40 | | | F127@PDA_60 | | | F127@PDA_100 | | |
|---|---|---|---|---|---|---|---|---|---|
| Disper- | Z-avg | | ζ | Z-avg | | ζ | Z-avg | | ζ |
| sant | (nm) | PDI | (mV) | (nm) | PDI | (mV) | (nm) | PDI | (mV) |
| Water | 61.3 ± 1.5 | 0.044 | −16.5 ± 1.1 | 83.3 ± 2.2 | 0.017 | −17.3 ± 2.7 | 116.2 ± 1.9 | 0.016 | −19.0 ± 1.3 |
| PBS 4.5 | 64.9 ± 0.9 | 0.096 | −6.7 ± 0.9 | 85.1 ± 1.7 | 0.029 | −8.4 ± 0.7 | 119.2 ± 2.2 | 0.008 | −5.3 ± 1.9 |
| PBS 6.5 | 69.9 ± 1.6 | 0.075 | −4.9 ± 0.3 | 83.8 ± 1.1 | 0.049 | −4.9 ± 1.8 | 120.4 ± 0.5 | 0.040 | −8.4 ± 1.4 |
| PBS 7.5 | 67.6 ± 1.6 | 0.066 | −5.2 ± 1.7 | 86.3 ± 0.5 | 0.050 | −5.3 ± 1.2 | 114.9 ± 2.9 | 0.035 | −6.4 ± 1.7 |
| PBS 8.5 | 70.4 ± 0.7 | 0.092 | −6.1 ± 0.5 | 84.6 ± 1.3 | 0.036 | −7.7 ± 0.3 | 126.9 ± 1.3 | 0.060 | −4.7 ± 0.3 |
| DMEM | 71.4 ± 2.1 | 0.069 | −4.8 ± 0.6 | 86.4 ± 2.1 | 0.044 | −4.5 ± 0.6 | 121.6 ± 1.5 | 0.076 | −6.4 ± 0.7 |
| DMEM | 68.1 ± 4.4 | 0.296 | −5.4 ± 0.2 | 82.6 ± 5.1 | 0.160 | −6.3 ± 1.9 | 116.2 ± 3.2 | 0.139 | −5.0 ± 2.1 |
| 10% FBS | | | | | | | | | |

### Synthesis of Fluorescein-TEG-NH₂ and Fluorescently Labelled F127@PDA

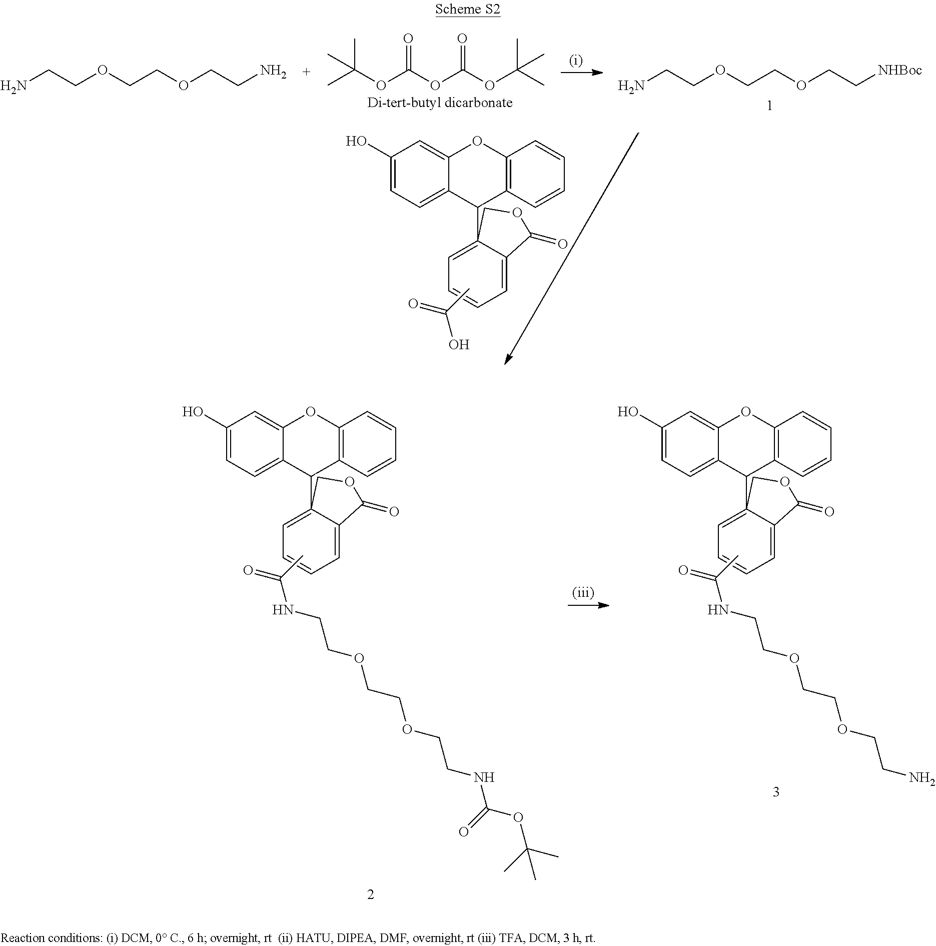

N-Boc-2,2′-(ethylene-I,2-dioxy)bisethylamine (1). Compound 1 was synthesized according to a reported method with slight modification.^{[99]} A solution of di-tert-butyl dicarbonate (11.0 g, 60.0 mmol) in 250 mL CH₂Cl₂ was added dropwise to a solution of 2,2′-(ethylenedioxy)bis(ethylamine) (30.0 mL, 200 mmol) in 200 mL dry CH₂Cl₂ at 0° C. under nitrogen atmosphere over a period of 6 h. The reaction mixture was stirred at 0° C. for 6 h and then at room temperature overnight. The mixture was extracted with 200 mL brine three times and 200 mL water. The organic phase was collected and dried over Na₂SO₄. The solvent was evaporated under vacuum to a give colourless oil (6.1 g, 71%). ¹H NMR (400 MHz, CDCl₃): δ (ppm) 1.41 (s, 9H). 2.61 (t, J=5.3 Hz, 2H), 3.02 (m, 2H), 3.24-3.36 (m, 4H), 3.38-3.44 (m, 4H), 5.41 (br, 1H). HR-MS (ESI): m/z [M⁺] calculated for C₁₁H₂₄N₂O₄: 248.1713; found: 248.1728.

Tert-butyl (2-(2-(2-acetamidoethoxy)ethoxy)ethyl)carbamate-3′,6′-dihydroxy-3H-spiro[isobenzofuran-1,9′-xanthen]-3-one (2). 5(6)-carboxyfluorescein (1.0 g, 2.65 mmol) was dissolved in anhydrous DMF (15 mL) under argon and HATU (1.22 g, 3.20 mmol) and DIPEA (1.029 g, 1.4 mL, 7.98 mmol) were added to the solution. The reaction mixture was stirred at room temperature under argon for 30 min. The solution of N-Boc-2,2′-(ethylene-1,2-dioxy)bisethylamine (1) (0.86 g, 3.45 mmol) in anhydrous DMF (5 mL) was slowly added under Ar. The reaction mixture was stirred overnight at room temperature. The solvent was removed under reduced pressure to obtain dark orange residue. Silica gel column chromatography using CH₂Cl₂:MeOH (9:1) gave pure compound 2 as orange thick oil (1.4 g, 2.31 mmol, 87% yield). ¹H NMR (400 MHz, DMSO-d₆, mixture of isomers): δ (ppm) 1.35-1.38 (m, 6H), 1.41-1.44 (m, 12H), 3.13 (t, J=7.1 Hz, 2H), 3.29-3.31 (m, 4H), 3.36-3.38 (m, 2H), 3.42-3.45 (m, 4H), 3.58-3.60 (m, 6H), 3.65-3.71 (m, 6H), 3.72 (bs, 2H), 6.51-6.55 (m, 4H, Ar—H), 6.57-6.59 (m, 2H, Ar—H), 6.70-6.75 (m, 4H, Ar—H), 7.24 (d, J=7.8 Hz, 2H, Ar—H), 7.33 (s, 1H, Ar—H), 7.60 (s, 1H, Ar—H), 8.05-8.11 (m, 3H, Ar—H), 8.23 (d, J=8.1 Hz, 1H, Ar—H), 8.45 (br s, 1H, NH). ¹³C NMR (100 MHz, DMSO-d₆, mixture of isomers): δ (ppm) 23.5, 26.7, 31.8, 33.7, 35.2, 36.6, 50.0, 65.5, 74.5, 79.1, 79.3, 82.4, 98.0, 98.1, 104.7, 107.8, 118.0, 118.8, 119.2, 120.3, 122.5, 124.1, 124.4, 128.5, 128.6, 128.7, 130.1, 131.6, 136.0, 147.8, 147.9, 151.6, 151.7, 159.2, 161.7, 164.5. HR-MS (ESI): m/z [M⁺] calculated for C₃₂H₃₄N₂O₁₀: 606.2245; found: 606.2214.

N-(2-(2-(2-aminoethoxy)ethoxy)ethyl)acetamide-3′,6′-dihydroxy-3H-spiro[isobenzofuran-1,9′-xanthen]-3-one (3). To a solution of Boc-protected compound 2 (500 mg, 0.825 mmol) in CH₂Cl₂ (20 mL) was added trifluoroacetic acid (6 mL). The mixture was stirred for 3 hours at room temperature. The solvent was evaporated under reduced pressure to give yellow residue. CH₂Cl₂ (20 mL) was added to the residue and evaporated. This process was repeated three times (3×20 mL) to remove the trifluoroacetic acid. Toluene (30 mL) was added to the residue and the solvent was evaporated in order to remove the traces of trifluoroacetic acid to give dark orange liquid of compound 3 amine as its trifluoroacetate salt (360 mg, 0.711 mmol, 86% yield). ¹H NMR (400 MHz, DMSO-d₆, mixture of isomers): δ (ppm) 2.85-2.90 (m, 2H), 2.92-2.96 (m, 2H), 3.05-3.11 (m, 2H), 3.29-3.35 (m, 2H), 3.35-3.45 (m, 2H), 3.47-3.49 (m, 2H), 3.55-3.61 (m, 6H), 3.62-3.65 (m, 6H), 6.49-6.58 (m, 4H, Ar—H), 6.61-6.69 (m, 3H, Ar—H), 7.05-7.10 (m, 2H, Ar—H), 7.19-7.24 (m, 2H, Ar—H), 7.35 (d, J=8.1 Hz, 1H, Ar—H), 7.64 (s, 1H, Ar—H), 7.66 (s, 1H, Ar—H), 8.03-8.16 (m, 3H, Ar—H), 8.23 (d, J=8.1 Hz, 1H, Ar—H), 8.41 (s, 1H). 8.72 (br s, 1H, NH). ¹³C NMR (100 MHz, DMSO-d₆, mixture of isomers): δ (ppm) 36.2, 42.2, 54.01, 67.0, 67.1, 68.7, 69.8, 70.0, 70.1, 79.1, 79.2, 102.7, 108.4, 108.5, 113.1, 114.4, 117.3, 120.2, 126.7, 128.6, 129.3, 129.5, 129.7, 135.1, 136.5, 137.8, 152.2, 150.2, 159.0, 160.1, 162.7, 165.0, 165.2, 168.5, 168.6. HR-MS (ESI): m/z [M⁺] calculated for C₂₇H₂₆N₂O₈: 506.1724; found: 506.1735.

### Fluorescein Functionalization of F127@PDA NPs

TABLE S3
DLS and zeta potential measurements of F127@PDA samples
before and after functionalization with Fluoresein-TEG-NH₂.
Errors are standard deviations of the triplicate data.

| | Hydrodynamic | | ζ |
|---|---|---|---|
| Sample | size (nm) | PDI | (mV) |
| F127@PDA_40 | 59.2 ± 0.9 | 0.027 | −18.3 ± 2.1 |
| F127@PDA@FI_40 | 71.6 ± 1.7 | 0.116 | −8.8 ± 2.1 |
| F127@PDA_60 | 75.2 ± 1.1 | 0.040 | −13.3 ± 0.7 |
| F127@PDA@FI_60 | 79.4 ± 3.2 | 0.132 | −11.6 ± 0.5 |
| F127@PDA_100 | 117.4 ± 2.2 | 0.016 | −14.6 ± 1.6 |
| F127@PDA@FI_100 | 123.6 ± 1.6 | 0.128 | −9.4 ± 1.3 |

Results of in Vitro evaluation of F127@PDA NPs are shown in FIG. 18.

Cytotoxicity study results are shown in FIG. 19.

Cell internalization study results are shown in FIG. 20.

Immunomodulation study results are shown in FIG. 22.

| | Live-cell imaging | | | | MTS | | | |
|---|---|---|---|---|---|---|---|---|
| | | | SN38@F127@ | | | | SN38@F127@ | |
| | SN38 | | PDA | | SN38 | | PDA | |
| | IC₅₀ | | IC₅₀ | | IC₅₀ | | IC₅₀ | |
| Cell line | (nM) | R² | (nM) | R² | (nM) | R² | (nM) | R² |
| AsPC-1 | 52.92 | 0.982 | 2.87 | 0.959 | 109.8 | 0.936 | 48.44 | 0.955 |
| BxPc-3 | 8.91 | 0.977 | 0.45 | 0.967 | 55.96 | 0.986 | 11.37 | 0.962 |
| MIA PaCa-2 | 12.11 | 0.908 | 0.18 | 0.844 | 13.10 | 0.982 | 2.71 | 0.888 |
| PANC-1 | 56.13 | 0.900 | 7.82 | 0.978 | 112.8 | 0.770 | 75.7 | 0.718 |

### References to Part 1 of Examples

[1] R. L. Siegel, K. D. Miller, A. Jemal, CA. Cancer J. Clin. 2018, 68, 7.
[2] “Pancreatic cancer statistics|Cancer Research UK,” can be found under https://www.cancerresearchuk.org/health-professional/cancer-statistics/statistics-by-cancer-type/pancreatic-cancer #heading-One, n.d.
[3] T. Kamisawa, L. D. Wood, T. Itoi, K. Takaori, Lancet 2016, 388, 73.
[4] J. Kleeff, M. Korc, M. Apte, C. La Vecchia, C. D. Johnson, A. V. Biankin, R. E. Neale, M. Tempero, D. A. Tuveson, R. H. Hruban, et al., Nat. Rev. Dis. Prim. 2016, 2, 16022.
[5] T. Su, B. Yang, T. Gao, T. Liu, J. Li, Ther. Adv. Med. Oncol. 2020, 12, DOI 10.1177/1758835920915978.
[6] T. Murakami, Y. Hiroshima, R. Matsuyama, Y. Homma, R. M. Hoffman, I. Endo, Ann. Gastroenterol. Surg. 2019, 3, 130.
[7] H. xiang Zhan, B. Zhou, Y. gang Cheng, J. wei Xu, L. Wang, G. yong Zhang, S. yuan Hu, Cancer Lett. 2017, 392, 83.
[8] M. Germain, F. Caputo, S. Metcalfe, G. Tosi, K. Spring, A. K. O. Åslund, A. Pottier, R. Schiffelers, A. Ceccaldi, R. Schmid, J. Control. Release 2020, 326, 164.
[9] M. R. C. Marques, Q. Choo, M. Ashtikar, T. C. Rocha, S. Bremer-Hoffmann, M. G. Wacker, Adv. Drug Deliv. Rev. 2019, 151-152, 23.
[10] A. C. Anselmo, S. Mitragotri, Bioeng. Transl. Med. 2019, 4, DOI 10.1002/btm2.10143.
[11] H. Meng, M. Wang, H. Liu, X. Liu, A. Situ, B. Wu, Z. Ji, C. H. Chang, A. E. Nel, ACS Nano 2015, 9, 3540.
[12] F. C. Passero, D. Grapsa, K. N. Syrigos, M. W. Saif, Expert Rev. Anticancer Ther. 2016, 16, 697.
[13] M. Au, T. I. Emeto, J. Power, V. N. Vangaveti, H. C. Lai, Biomedicines 2016, 4, DOI 10.3390/biomedicines4030020.
[14] L. Zhu, C. Staley, D. Kooby, B. El-Rays, H. Mao, L. Yang, Cancer Lett. 2017, 388, 139.
[15] J. I. Hare, T. Lammers, M. B. Ashford, S. Puri, G. Storm, S. T. Barry, Adv. Drug Deliv. Rev. 2017, 108, 25.
[16] K. Park, J. Control. Release 2019, 305, 221.
[17] K. Park, J. Control. Release 2014, 190, 3.
[18] D. Sun, S. Zhou, W. Gao, ACS Nano 2020, 14, 12281.
[19] S. Wilhelm, A. J. Tavares, Q. Dai, S. Ohta, J. Audet, H. F. Dvorak, W. C. W. Chan, Nat. Rev. Mater. 2016, 1, 1.
[20] Y. H. Bae, K. Park, J. Control. Release 2011, 153, 198.
[21] G. Hannon, J. Lysaght, N. J. Liptrott, A. Prina-Mello, Adv. Sci. 2019, 6, 1900133.
[22] I. Mottas, A. Milosevic, A. Petri-Fink, B. Rothen-Rutishauser, C. Bourquin, Nanoscale 2017, 9, 2492.
[23] M. A. Dobrovolskaia, D. R. Germolec, J. L. Weaver, Nat. Nanotechnol. 2009 47 2009, 4, 411.
[24] M. Elsabahy, K. L. Wooley, Chem. Soc. Rev. 2013, 42, 5552.
[25] M. Durymanov, C. Kroll, A. Permyakova, J. Reineke, Mol. Pharm. 2019, 16, 1074.
[26] H. Lee, H. Fonge, B. Hoang, R. M. Reilly, C. Allen, Mol. Pharm. 2010, 7, 1195.
[27] M. Danaei, M. Dehghankhold, S. Ataei, F. Hasanzadeh Davarani, R. Javanmard, A. Dokhani, S. Khorasani, M. R. Mozafari, Pharmaceutics 2018, 10, 57.
[28] J. Wang, W. Mao, L. L. Lock, J. Tang, M. Sui, W. Sun, H. Cui, D. Xu, Y. Shen, ACS Nano 2015, 9, 7195.
[29] Z. Amoozgar, Y. Yeo, Wiley Interdiscip. Rev. Nanomedicine Nanobiotechnology 2012, 4, 219.
[30] S. Mishra, P. Webster, M. E. Davis, Eur. J. Cell Biol. 2004, 83, 97.
[31] J. J. F. Verhoef, J. F. Carpenter, T. J. Anchordoquy, H. Schellekens, Drug Discov. Today 2014, 19, 1945.
[32] R. P. Garay, R. El-Gewely, J. K. Armstrong, G. Garratty, P. Richette, Expert Opin. Drug Deliv. 2012, 9, 1319.
[33] A. Pitto-Barry, N. P. E. Barry, Polym. Chem. 2014, 5, 3291.
[34] E. V. Batrakova, A. V. Kabanov, J. Control. Release 2008, 130, 98.
[35] J. G. Dancy, A. S. Wadajkar, C. S. Schneider, J. R. H. Mauban, O. G. Goloubeva, G. F. Woodworth, J. A. Winkles, A. J. Kim, J. Control. Release 2016, 238, 139.
[36] E. A. Nance, G. F. Woodworth, K. A. Sailor, T. Y. Shih, Q. Xu, G. Swaminathan, D. Xiang, C. Eberhart, J. Hanes, Sci. Transl. Med. 2012, 4, 149ra119.
[37] R. P. Mohanty, X. Liu, J. Y. Kim, X. Peng, S. Bhandari, J. Leal, D. Arasappan, D. C. Wylie, T. Dong, D. Ghosh, Nanoscale 2019, 11, 17664.
[38] J. W. Valle, A. Armstrong, C. Newman, V. Alakhov, G. Pietrzynski, J. Brewer, S. Campbell, P. Corrie, E. K. Rowinsky, M. Ranson, Invest. New Drugs 2011, 29, 1029.
[39] A. V. Kabanov, E. V. Batrakova, V. Y. Alakhov, Adv. Drug Deliv. Rev. 2002, 54, 759.
[40] B. Y. Guan, L. Yu, X. W. Lou, J. Am. Chem. Soc. 2016, 138, 11306.
[41] F. Chen, Y. Xing, Z. Wang, X. Zheng, J. Zhang, K. Cai, Langmuir 2016, 32, 12119.
[42] C. O. Franck, L. Fanslau, A. Bistrovic Popov, P. Tyagi, L. Fruk, Angew. Chemie Int. Ed. 2021, anie. 202010282.
[43] S. K. Nitta, K. Numata, Int. J. Mol. Sci. 2013, 14, 1629.
[44] W. Cao, X. Zhou, N. C. McCallum, Z. Hu, Q. Z. Ni, U. Kapoor, C. M. Heil, K. S. Cay, T. Zand, A. J. Mantanona, et al., J. Am. Chem. Soc. 2021, 143, 2622.
[45] S. Sunoqrot, N. N. Mahmoud, L. H. Ibrahim, S. Al-Dabash, H. Raschke, R. Hergenroder, ACS Biomater. Sci. Eng. 2020, 6, 4424.
[46] K. Y. Ju, Y. Lee, S. Lee, S. B. Park, J. K. Lee, Biomacromolecules 2011, 12, 625.
[47] Y. Liu, K. Ai, J. Liu, M. Deng, Y. He, L. Lu, Adv. Mater. 2013, 25, 1353.
[48] Y. Yue, F. Li, Y. Li, Y. Wang, X. Guo, Z. Cheng, N. Li, X. Ma, G. Nie, X. Zhao, ACS Nano 2021, acsnano.1c05618.
[49] I. Zmerli, N. Ibrahim, P. Cressey, S. Denis, A. Makky, Mol. Pharm. 2021, 18, 3623.
[50] H. G. Derami, P. Gupta, K.-C. Weng, A. Seth, R. Gupta, J. R. Silva, B. Raman, S. Singamaneni, Adv. Mater. 2021, 33, 2008809.
[51] L. P. Ferreira, V. M. Gaspar, M. V. Monteiro, B. Freitas, N. J. O. Silva, J. F. Mano, J. Control. Release 2021, 331, 85.
[52] L. Zhang, P. Yang, R. Guo, J. Sun, R. Xie, W. Yang, Int. J. Nanomedicine 2019, 14, 8647.
[53] R. Liu, F. Xu, L. Wang, M. Liu, X. Cao, X. Shi, R. Guo, Nanomater. 2021, Vol. 11, Page 394 2021, 11, 394.
[54] X. Z, Z. W, C. L, W. L, L. N, L. J, J. Q, Z. Y, Z. X, ACS Appl. Mater. Interfaces 2021, acsami.1c10341.
[55] Q. Jiang, M. Pan, J. Hu, J. Sun, L. Fan, Z. Zou, J. Wei, X. Yang, X. Liu, Chem. Sci. 2021, 12, 148.
[56] M. Li, W. Jiang, Z. Chen, S. Suryaprakash, S. Lv, Z. Tang, X. Chen, K. W. Leong, Lab Chip 2017, 17, 635.
[57] Y. Liu, S. Fu, L. Lin, Y. Cao, X. Xie, H. Yu, M. Chen, H. Li, Int. J. Nanomedicine 2017, 12, 2635.
[58] S. Harvey, D. Yuen, W. Ng, J. Szelwicka, L. Hueske, L. Veith, M. Raabe, I. Lieberwirth, G. Fytas, K. Wunderlich, et al., Biointerphases 2018, 13, 6.
[59] Leander Crocker, Philipp Koehler, Patrick Bernhard, Antonina Kerbs, Tijmen Euser, Ljiljana Fruk, Nanoscale Horizons 2019, 4, 1318.
[60] Qin Yue, Minghong Wang, Zhenkun Sun, Chun Wang, Can Wang, Yonghui Deng, Dongyuan Zhao, J. Mater. Chem. B 2013, 1, 6085.
[61] E. Herlinger, R. F. Jameson, W. Linert, J. Chem. Soc. Perkin Trans. 2 1995, 259.
[62] X. Jiang, Y. Wang, M. Li, Sci. Reports 2014 412014, 4, 1.
[63] J. Yan, L. Yang, M.-F. Lin, J. Ma, X. Lu, P. S. Lee, Small 2013, 9, 596.
[64] V. Francia, K. Yang, S. Deville, C. Reker-Smit, I. Nelissen, A. Salvati, ACS Nano 2019, 13, 11107.
[65] C. D. Walkey, J. B. Olsen, H. Guo, A. Emili, W. C. W. Chan, J. Am. Chem. Soc. 2012, 134, 2139.
[66] Y. Wei, L. Gao, L. Wang, L. Shi, E. Wei, B. Zhou, L. Zhou, B. Ge, Drug Deliv 2017, 24, 681.
[67] S. H, X. G, Y. C, W. Y, Z. A, H. N, L. J, J. Biomater. Sci. Polym. Ed. 2017, 28, 2066.
[68] F. Minami, N. Sasaki, Y. Shichi, F. Gomi, M. Michishita, K. Ohkusu-Tsukada, M. Toyoda, K. Takahashi, T. Ishiwata, Sci. Rep. 2021, 11, 6775.
[69] J. Peng, B.-F. Sun, C.-Y. Chen, J.-Y. Zhou, Y.-S. Chen, H. Chen, L. Liu, D. Huang, J. Jiang, G.-S. Cui, et al., Cell Res. 2019, 29, 725.
[70] E. L. Deer, J. González-Hernández, J. D. Coursen, J. E. Shea, J. Ngatia, C. L. Scaife, M. A. Firpo, S. J. Mulvihill, Pancreas 2010, 39, 425.
[71] J. Cicenas, K. Kvederaviciute, I. Meskinyte, E. Meskinyte-Kausiliene, A. Skeberdyte, J. Cicenas, Jr., Cancers (Basel). 2017, 9, DOI 10.3390/CANCERS9050042.
[72] A. Lesniak, A. Salvati, M. J. Santos-Martinez, M. W. Radomski, K. A. Dawson, C. Aberg, J. Am. Chem. Soc. 2013, 135, 1438.
[73] J. Mosquera, I. Garcia, L. M. Liz-Marzán, Acc. Chem. Res. 2018, 51, 2305.
[74] S. Behzadi, V. Serpooshan, W. Tao, M. A. Hamaly, M. Y. Alkawareek, E. C. Dreaden, D. Brown, A. M. Alkilany, O. C. Farokhzad, M. Mahmoudi, Chem. Soc. Rev. 2017, 46, 4218.
[75] H. R. Shin, M. Kwak, T. G. Lee, J. Y. Lee, Nanoscale 2020, 12, 15743.
[76] O. Lunov, T. Syrovets, C. Loos, J. Beil, M. Delacher, K. Tron, G. U. Nienhaus, A. Musyanovych, V. Mailänder, K. Landfester, et al., ACS Nano 2011, 5, 1657.
[77] T. dos Santos, J. Varela, I. Lynch, A. Salvati, K. A. Dawson, Small 2011, 7, 3341.
[78] James Lazarovits, Y. Yang Chen, E. A. Sykes, W. C. W. Chan, Chem. Commun. 2015, 51, 2756.
[79] A. B. Engin, A. W. Hayes, https://doi.org/10.1177/2397847318755579 2018, 2, 239784731875557.
[80] I. Gaz̆ová, L. Lefevre, S. J. Bush, S. Clohisey, E. Arner, M. de Hoon, J. Severin, L. van Duin, R. Andersson, A. Lengeling, et al., Front. Cell Dev. Biol. 2020, 0, 498.
[81] G. Kleveta, K. Borzpcka, M. Zdioruk, M. Czerkies, H. Kuberczyk, N. Sybirna, A. Sobota, K. Kwiatkowska, J. Cell. Biochem. 2012, 113, 80.
[82] A. A. Mohammad, Oncol. Rev. 2018, 12, 98.
[83] M. Capello, M. Lee, H. Wang, I. Babel, M. H. Katz, J. B. Fleming, A. Maitra, H. Wang, W. Tian, A. Taguchi, et al., JNCI J. Natl. Cancer Inst. 2015, 107, 132.
[84] V. Charasson, R. Bellott, D. Meynard, M. Longy, P. Gorry, J. Robert, Clin. Pharmacol. Ther. 2004, 76, 528.
[85] P. Tobin, S. Clarke, J. P. Seale, S. Lee, M. Solomon, S. Aulds, M. Crawford, J. Gallagher, T. Eyers, L. Rivory, Br. J. Clin. Pharmacol. 2006, 62, 122.
[86] Y. Kawato, M. Aonuma, Y. Hirota, H. Kuga, K. Sato, Cancer Res. 1991, 51.
[87] T. Hamaguchi, T. Doi, T. Eguchi-Nakajima, K. Kato, Y. Yamada, Y. Shimada, N. Fuse, A. Ohtsu, S. Matsumoto, M. Takanashi, et al., Clin. Cancer Res. 2010, 16, 5058.
[88] L. Wang, X. Liu, Q. Zhou, M. Sui, Z. Lu, Z. Zhou, J. Tang, Y. Miao, M. Zheng, W. Wang, et al., Biomaterials 2017, 144, 105.
[89] V. Bala, S. Rao, B. J. Boyd, C. A. Prestidge, J. Control. Release 2013, 172, 48.
[90] X. Wang, J. Zhang, Y. Wang, C. Wang, J. Xiao, Q. Zhang, Y. Cheng, Biomaterials 2016, 81, 114.
[91] Z. Wang, Y. Duan, Y. Duan, J. Control. Release 2018, 290, 56.
[92] T. Xuan, J. A. Zhang, I. Ahmad, J. Pharm. Biomed. Anal. 2006, 41, 582.
[93] “Home page—Cancerrxgene—Genomics of Drug Sensitivity in Cancer,” can be found under https://www.cancerrxgene.org/, n.d.
[94] M. Hazekawa, T. Nishinakagawa, T. Kawakubo-Yasukochi, M. Nakashima, Exp. Ther. Med. 2019, 18, 3197.
[95] N. S. Ng, M. J. Wu, S. J. Myers, J. R. Aldrich-Wright, J. Inorg. Biochem. 2018, 179, 97.
[96] I. Arany, R. L. Safirstein, Semin. Nephrol. 2003, 23, 460.
[97] G. H. Zhu, M. Azharuddin, R. Islam, H. Rahmoune, S. Deb, U. Kanji, J. Das, J. Osterrieth, P. Aulakh, H. Ibrahim-Hashi, et al., ACS Appl. Mater. Interfaces 2021, 13, 23410.
[98] S. Shen, Y. Wu, Y. L. Liu, D. Wu, Int. J. Nanomedicine 2017, 12, 4085.
[99] Z. Zeng, S. Mizukami, K. Kikuchi, Anal. Chem. 2012, 84, 9089.
[100] W. Yang, J. Soares, P. Greninger, E. J. Edelman, H. Lightfoot, S. Forbes, N. Bindal, D. Beare, J. A. Smith, I. R. Thompson, et al., Nucleic Acids Res. 2013, 41, D955.

### Part 2

1. F127@PDA Post-Functionalization with Rhodamine-TEG-NH₂ (Alternative Dye to Fluorescein) and Small Peptides Containing Cysteine (Thiol) Residues (TAMRA-Labelled-NLS Peptide and ATRA-Functionalized MMP Cleavable Peptide)

### Description

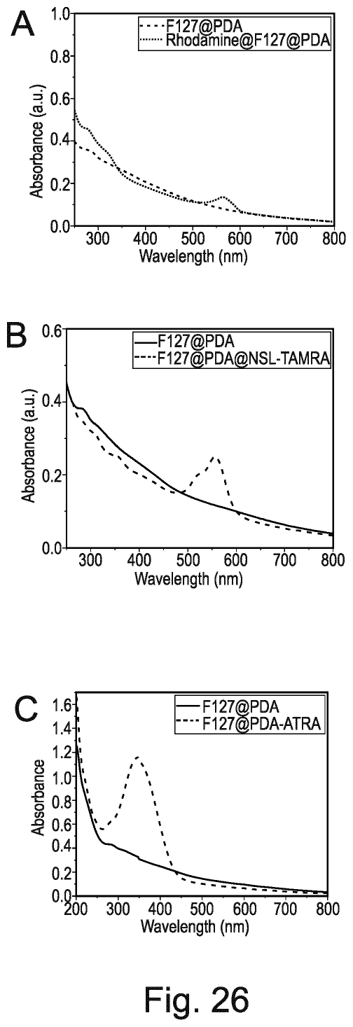

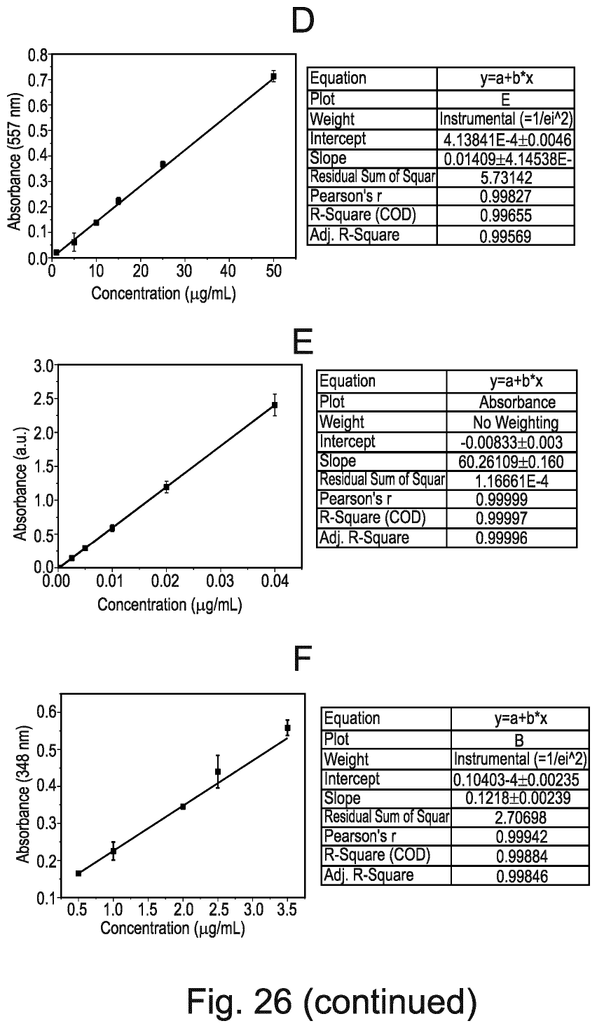

Post-functionalization of F127@PDA NPs was conducted via Michael addition with amino-functionalized Rhodamine-TEG-NH₂ (4) and thiol/cysteine containing small peptides, such as nuclear localization sequence (NLS) SV40 labelled with TAMRA (5) and all-trans retinoic acid (ATRA) functionalized MMP cleavable peptide sequence (6) (Scheme 1). The successful modification was validated through detection of the characteristic rhodamine, TAMRA or ATRA peak in the absorbance spectra (FIG. 26). The loading content (w/w) of the functionalized materials was determined by UV-Vis and was found to be 4% for Rhodamine-TEG-NH₂, 3% for TAMRA-NLS and 6% for ATRA-MMP linkers. A slight increase in hydrodynamic sizes was observed after functionalization of the NPs, and the introduction of positively charged NLS ligand 5 was also confirmed by the shift in zeta potential after functionalization (Table 1).

TABLE 1
Hydrodynamic diameter (Z-average), polydispersity index (PDI) and zeta potential
(ζ) of F127@PDA NPs before and after functionalization with ligands 4-6.

| | Z-average | | ζ |
|---|---|---|---|
| | (nm) | PDI | (mV) |
| F127@PDA | 106.2 ± 0.8 | 0.088 ± 0.009 | −14.6 ± 1.2 |
| TAMRA- | 117.4 ± 1.9 | 0.016 ± 0.008 | 23.7 ± 2.5 |
| NLS@F127@PDA | | | |
| F127@PDA | 89.7 ± 1.1 | 0.059 ± 0.020 | −17.3 ± 0.8 |
| ATRA-MMP@F127@PDA | 125.3 ± 0.6 | 0.111 ± 0.012 | −19.2 ± 2.1 |
| F127@PDA | 122.2 ± 1.9 | 0.008 ± 0.016 | −22.4 ± 1.4 |
| Rhodamine@F127@PDA | 134.12 ± 3.2 | 0.186 ± 0.032 | —^{a} |

^{a}not determined

### Methods

Ligand 2 (TAMRA NLS SV40, TAMRA-CONH—PKKKRKVC—COOH) (SEQ. ID 15) and MMP cleavable peptide sequence (NH₂-QGAIGLPGC—COOH) (SEQ. ID 16) were purchased from BioServUK and all-trans retinoic acid was purchased from ChemCruz Biotechnology (US).

### General Procedure for the Post-Functionalization of F127@PDA NPs Via Michael Addition

F127@PDA NPs (5 mg) was dissolved in 10 mM Tris buffer (5 mL) and the corresponding ligand 4-6 (10 mg, 2 wt eq.) dissolved in 0.5 mL ethanol was added dropwise. The mixture was protected from direct sunlight and stirred over night at room temperature. Excess ligand was removed washing with Milli-Q water using Vivaspin 20 (Satorius, UK) centrifugal concentrators (10 kDa MWCO) for 15 min at 4000 rpm until the supernatant was colorless, followed by 3 days dialysis against water with 12-14 kDa MWCO dialysis bags.

### Synthesis of Rhodamine-TEG-NH₂

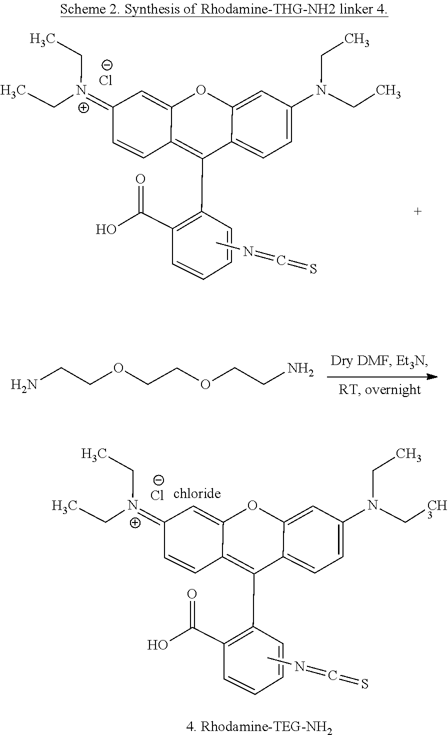

### Synthesis of Ligand 1

2,2′-(ethylenedioxy)bis(ethylamine) (110 mg, 0.930 mmol) was dissolved in anhydrous DMF (10 mL) in an oven dried 2-neck flask under argon and triethylamine (56 mg, 76 μL, 0.560 mmol) was added. The solution was cooled with ice bath and solution of Rhodamine B isothiocyanate (mixed isomers) (100 mg, 0.186 mmol) in DMF (10 mL) was added to the solution dropwise over a period of 30 min. After complete addition, the ice bath was removed and the reaction mixture was stirred overnight at room temperature. The solvent was removed under reduced pressure to give dark purple residue which were used without further purification. ¹H NMR (400 MHz, CDCl₃, mixture of isomers): δ (ppm) 1.15 (t, J=7.1 Hz, 6H), 1.27 (t, J=7.1 Hz, 6H), 1.80 (m, 4H) 3.09 (br, 2H), 3.26-3.37 (m, 8H), 3.45-373 (m, 28H), 6.28 (d, J=1.7 Hz, 1H), 6.38 (dd, J=2.4 Hz, 8.8 Hz, 2H), 6.41 (d, J=2.4 Hz, 1H), 6.65 (br, 2H), 6.70-7-75 (m, 3H), 6.83 (d, J=9.3 Hz 2H), 6.96 (d, J=8.3 Hz, 1H), 7.31 (d, J=9.3 Hz, 2H), 7.71 (d, J=8.3 Hz, 1H), 7.80 (br, 1H), 8.22 (br, 1H), 8.81 (br, 2H). ¹³C NMR (100 MHz, CDCl₃, mixture of isomers): δ (ppm) 12.5, 12.6, 25.6, 28.4, 39.6, 40.2, 44.6, 45.7, 66.9, 67.9, 70.0, 70.2, 70.3, 95.7, 95.8, 97.2, 107.5, 107.7, 108.1, 108.6, 113.2, 113.9, 115.9, 117.9, 122.7, 126.9, 129.2, 129.4, 149.5, 149.9, 152.3, 153.6, 154.8, 156.1, 157.8, 162.7, 180.9. HRMS (ESI) for C₃₅H₄₆ClN₅O₅S [M+H]⁺: 684.21439

### Synthesis of ATRA-MMP Cleavable Peptide Linker

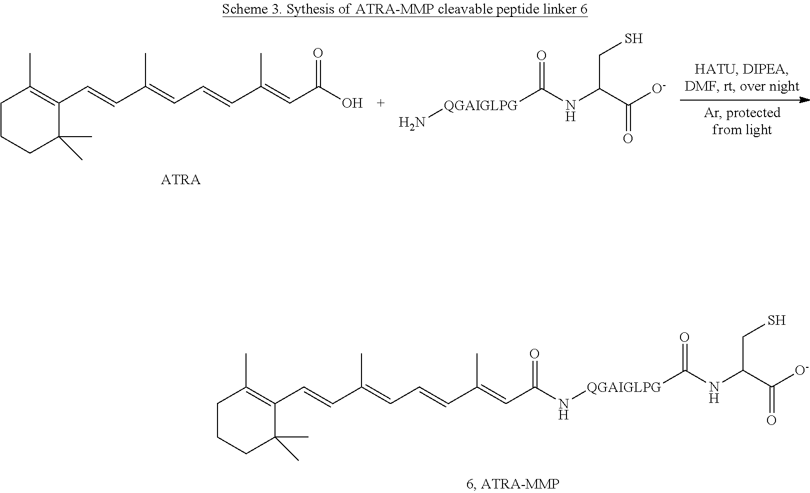

### Synthesis of Ligand 3

NH₂-QGAIGLPGC—COOH (SEQ. ID 17) peptide (25 mg, 0.03 mmol), ATRA (8.4 mg, 0.03 mmol), 1-[bis(dimethylamino)methylene]-1H-1,2,3-triazolo[4,5-b]pyridinium-3-oxide hexafluorophosphate (HATU; 14.14 mg, 0.037 mmol) and N,N-diisopropylethylamine (DIPEA; 0.01 mL, 0.062 mmol) were dissolved in dry DMF (5 mL). The reaction mixture was stirred overnight under inert atmosphere protected from direct sunlight. The solvent was removed by rotary evaporation and the resulting product purified using a flash column with DCM:MeOH (10:1) to yield yellow powder (30 mg, yield: 61%). HR-MS (ESI): m/z [M⁺] calculated for C₅₅H₈₇N₁₀O₁₂S: 1111.6226; found: 1111.6241.

### Non-Covalent Loading of Other Drugs Through Adsorption Paclitaxel (PTX) and Gemcitabine (Gem)

### Description

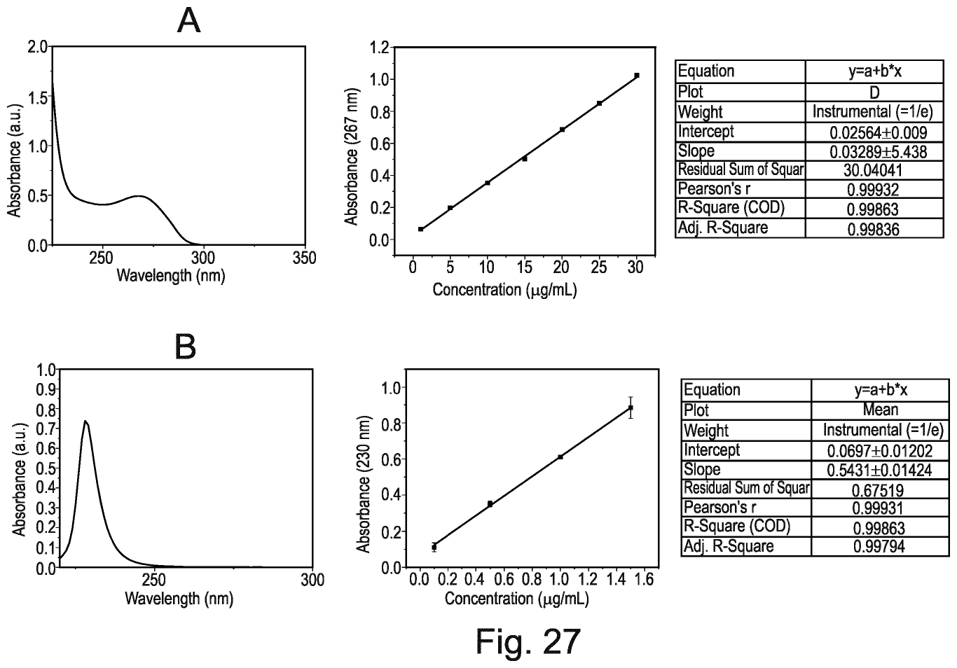

In addition to SN38, the possibility to encapsulate other standard of care drugs for pancreatic cancer (PTX and Gem) within F127@PDA NPs was evaluated (Scheme 4). Physical adsorption was achieved by mixing a suspension of PTX or Gem and F127@PDA NPs, a loading content of 14.3±4.7% (w/w) and 11.5±5.7% (w/w) was obtained from UV-Vis spectroscopy, respectively (FIG. 27). To optimize the loading procedure a semi-automated method using T-junction microfluidics mixer was used. The loading process using the microfluidic mixer showed similar loading content to the conventional method, but the reaction time was significantly shorter and the stability of the drug loaded NPs greater using the semi-automated method.

### Methods

Gemcitabine was purchased from Acros Organics and Paclitaxel from TCI.

### General Procedure for Drug Loading

A suspension of 10 mg F127@PDA NPs (1 mg/mL) and 2.5 mg of the corresponding drug (0.25 wt eq.) in DMSO:H₂O=1:10 (10 mL) were sonicated for 30 min, followed by stirring at room temperature for 72 hours. To remove the free drug, the reaction mixture was first centrifuged at 2000 rpm for 5 min. The supernatant was collected and washed with Milli-Q water using Vivaspin 20 (Satorius, UK) centrifugal concentrators (10 kDa MWCO) for 15 min at 4000 rpm. The obtained particles were diluted in Milli-Q water, frozen in liquid nitrogen and lyophilized to yield a dark brown powder. The loading content of Gem and PTX within the NPs was determined using UV-Vis. The absorbance of the free drug in the supernatant was measured and the loading content was calculated according to the following equation:

$$Loading content ( weight [ wt ] \% ) = \frac{Mass of drug in nanocarrier}{Initial mass of nanocarrier} \times 100$$

### General Procedure for the Drug Loading Using a Microfluidics Mixer

The T-junction Chip with Header was purchased from Dolomite (United Kingdom, Royston). The chip is made from glass with a hydrophobic coating, and it has a channel depth and width of 100 and 110 μm, respectively. The channel length after the junction is 278 mm. Linear Connector 4-way, PTFE Tubing with the outer and inner diameters of 1.6 and 0.5 mm, a 2-way In-line Valve, Female to Female Luer Lock, and End Fittings and Ferrules were also purchased from Dolomite. Linear Connector 4-way is used to connect the T-junction Chip to the PTFE Tubing, while Female to Female Luer Lock and End Fittings and Ferrules are used for Luer Lock syringes. The syringes are connected to two NE-300 Just Infusion™ Syringe Pumps ordered from New Era Pump System Inc (United States, New York). Note that 3 and 5 ml Luer Lock syringes are used in this experiment. Filters with a pore size of 0.2 μm are also employed to remove impurities in the inlet streams. A F127@PDA NP solution in EtOH (4 mg/mL, 5 mL) was injected to the main channel at a 10 μL/min flow rate and the corresponding drug solution in EtOH (2 mg/mL, 2.5 mL) into the side channel at a 5 μL/min flow rate. The solutions were mixed for 6 h. Ethanol was removed by rotary evaporation and the resulting mixture resuspended in 5 mL Milli-Q water followed by the same washing procedure as with the conventional loading method.

One-pot synthesis of ATRA@F127@PDA NPs. Covalent attachment of ATRA-modified with dopamine (during synthesis process, 3 constituent synthesis)

### Description

### ATRA@F127@PDA

NPs were prepared in a one-pot reaction trough co-polymerization of Pluronic-dopamine (F127DA) and ATRA-dopamine (ATRADA) monomers with dopamine (DA) in Tris-buffer (Scheme 5). ATRADA monomer was prepared in a two-step synthesis as shown in Scheme 6. Using this one-pot synthesis spherical particles were obtained with a hydrodynamic diameter of 98.96±1.3 nm, polydispersity index of 0.135±0.007 and zeta potential of −25.3±0.52. Based on the UV-Vis spectra a loading content of 9% wt ATRA in 1 mg of NPs was obtained.

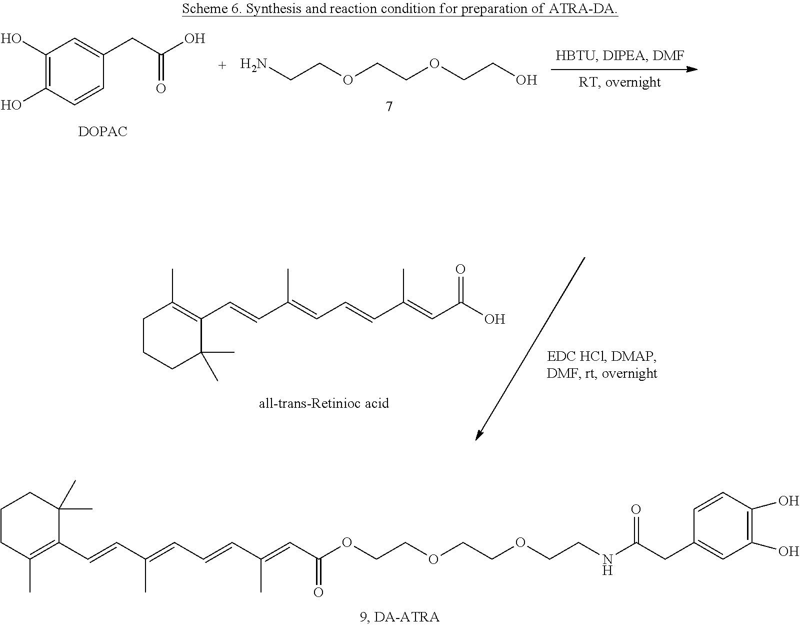 [SMILES: O=C(O)Cc1ccc(O)c(O)c1 ; NCCOCCOCCO ; CC1=C(/C=C/C(C)=C/C=C/C(C)=C/C(=O)O)C(C)(C)CCC1 ; CC1=C(/C=C/C(C)=C/C=C/C(C)=C/C(=O)OCCOCCOCCNC(=O)Cc2ccc(O)c(O)c2)C(C)(C)CCC1]

### Synthesis of 2-(3,4-dihydroxyphenyl)-N-(2-(2-(2-hydroxyethoxy)ethoxy)ethyl)acetamide (8)

Compound 7 was prepared starting from triethylene glycol in a three-step procedure according to a reported method.^{[1]} To a stirred solution of 2-(3,4-dihydroxyphenyl)acetic acid (DOPAC 1.0 g, 5.95 mmol, 1 equiv.) and HBTU (2.70 g, 7.14 mmol, 1.2 equiv.) in 30 mL DMF was added DIPEA (1.53 g, 2.05 mL, 11.9 mmol, 2 equiv). The reaction mixture was cooled to 0° C. with ice bath and a solution of compound 7 (1.06 g, 7.14 mmol, 1.2 equiv.) in 10 mL DMF was added dropwise over a period of 30 min. The ice bath was removed and the reaction mixture was stirred at room temperature overnight. The solvent was removed and the residue was dissolved in 50 mL CHCl₃ and washed with water (2×20 mL), brine (20 mL) and dried over anhydrous sodium sulphate. The combined organic layer was concentrated to give yellow oil which was purified by silica gel chromatography eluting with CH₂Cl₂ to CH₂Cl₂/MeOH (10:1) to afford compound 8 as pale-yellow liquid (yield: 77%). ¹H NMR (400 MHz, CDCl₃): δ (ppm) 3.41 (t, J=5.2 Hz, 2H), 3.44 (br, 2H), 3.49 (s, 2H), 3.51 (t, J=5.2 Hz, 2H), 6.54-6.58 (m, 2H), 6.60-6.64 (m, 2H), 3.78 (t, J=4.6 Hz, 2H), 6.12 (br, 2H), 6.63 (dd, J=2.0 Hz, 8.0 Hz, 1H), 6.75 (d, J=2.0 Hz, 1H), 3.81 (d, J=8.0 Hz, 1H), 8.01 (br, 1H); ¹³C NMR (100 MHz, CDCl₃): δ (ppm) 39.1, 42.7, 61.7, 70.0, 70.2, 70.5, 72.3, 115.1, 116, 121.9, 127.4, 143.9, 144.4, 172.6. HRMS (FAB) for C₁₄H₂₁NO₆ [M+H]⁺: 300.31735.

### 2-{2-{2-(2-(3,4-dihydroxyphenyl)acetamido)ethoxy]ethoxy}ethyl (2E,4E,6E,8E)-3,7-dimethyl-9-(2,6,6-trimethylcyclohex-1-en-1-yl)nona-2,4,6,8-tetraenoate (ATRA-DA, 9)

ATRA (250 mg, 0.83 mmol), 8 (299.3 mg, 0.83 mmol), N-ethyl-N′-(dimethylaminopropyl)carbodiimide hydrocholoride (EDC HCl; 159.1 mg, 0.83 mmol) and 4-dimethylaminopyridine (DMAP, 101.4 mg, 0.83 mmol) were dissolved in dry dichloromethane (20 mL). The reaction mixture was stirred overnight under inert atmosphere protected from direct sunlight. The solvent was removed by rotary evaporation and the resulting product purified using silica gel column chromatography using CH₂Cl₂:MeOH (10:1) to yield orange powder (349.6 mg, 72.5%). ¹H NMR (400 MHz, CDCl₃): δ (ppm) 1.03 (s, 6H). 1.45-1.51 (m, 2H), 1.57-1.66 (m, 2H), 1.72 (S, 3H), 1.98-3.06 (m, 5H), 2.40 (s, 3H), 3.38-3.45 (M, 2H), 3.46-3.66 (m, 8H), 3.68-3.77 (m, 2H), 4.15-4.29 (m, 2H), 6.04 (s, 1H), 6.12-6.26 (m, 4H), 6.34 (s, 1H), 6.78 (dd, J=8.2, 1.8 Hz, 1H), 6.93 (d, J=1.7 Hz, 1H), 7.03 (d, J=8.1 Hz, 1H). HR-MS (ESI): m/z [M⁺] calculated for C₃₄H₄₈NO₇: 582.3431; found: 581.3436.

### One-Pot Synthesis of ATRA@F127@PDA NPs (Almost the Same as F127@PDA NPs)

Trizma-base (22.5 mg) was dissolved in 2.5 mL Milli Q water and added to a mixture of ethanol (10.5 mL) and Milli-Q water (19.5 mL) and stirred for 30 min at room temperature. Dopamine hydrochloride (13.7 mg, 0.036 mmol) dissolved in 1 mL Milli Q water, F127DA (31.9 mg, 0.0024 mmol) dissolved in 1 mL ethanol and ATRADA (21.04 mg, 0.036 mmol) dissolved in 1 mL ethanol were mixed and sonicated before being added dropwise to the reaction mixture. The mixture was left to stir over night at room temperature resulting in a dark brown solution. The reaction mixture was washed with Milli-Q water using Vivaspin 20 (Satorius, UK) centrifugal concentrators (30 kDa MWCO) for 15 min at 4000 rpm until the supernatant was colorless. The obtained particles were diluted in Milli-Q water, frozen in liquid nitrogen and lyophilized to yield a dark brown powder.

### Synthesis of Pluronic-Pyrazoline-Polydopamine NPs (F127Py@PDA)—Covalent Attachment of a Fluorescent Group Between Dopamine and Pluronic, Giving a Modified F127DA Monomer Unite

### Description

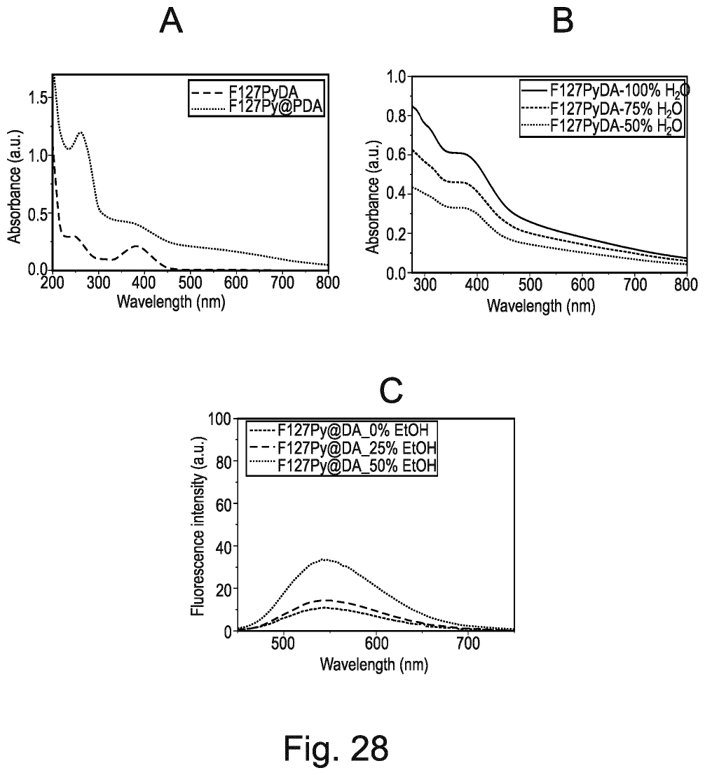

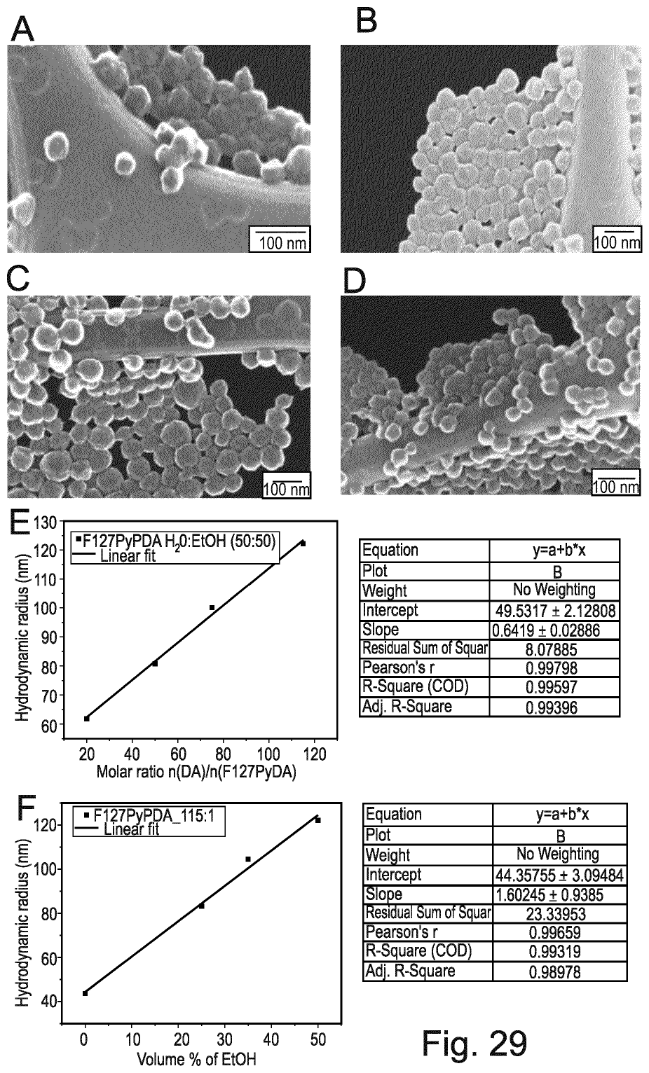

Fluorescent Pluronic-polydopamine (F127Py@PDA) nanoparticles were obtained by oxidative co-polymerization of the prepared Pluronic-pyrazoline-dopamine (F127-Py-DA) monomer and dopamine (DA) (Scheme 7). The fluorescent F127-Py-DA monomer was first synthesized according to Scheme 8. Briefly, maleimide-terminated Pluronic F127 (F127-Mal) was prepared according to a modified procedure given in literature^{[2]} in two consecutive steps; chlorination of maleimidobutiric acid to maleimidobutiric acyl chloride, which reacted with Pluronic F127. The nitrile imine-mediated tetrazol-ene cycloaddition (NITEC) was utilized to form the fluorescent pyrazoline product (F127-Py-DA). The photoreaction of tetrazole 4-(2-(4-methoxyphenyl)-2H-tetrazol-5-yl)benzoic acid and F127-Mal was monitored using fluorescence spectra and the characteristic broad emission spectra of pyrazoline with λₑₘ=560 at the excitation wavelength of λₑₓ=400 nm was noted (FIG. 28). Similar to the non-fluorescent F127@PDA NPs the pyrazoline linked F127Py@PDA show size-tunable properties trough adjustment of the ethanol ration or monomer ration in the reaction mixture (FIG. 29 and Table 2). Additionally, their ability to load drugs was demonstrated trough adsorption of SN-38 using the previously described conventional loading method and a loading content of 7.2% was observed.

### 3.2. Methods

4-(2-(4-methoxyphenyl)-2H-tetrazol-5-yl)benzoic acid^{[3]} and 4-(2,5-dioxo-2,5-dihydro-1H-pyrrol-1-yl)butanoic acid^{[4,5]} was prepared according to literature procedure.

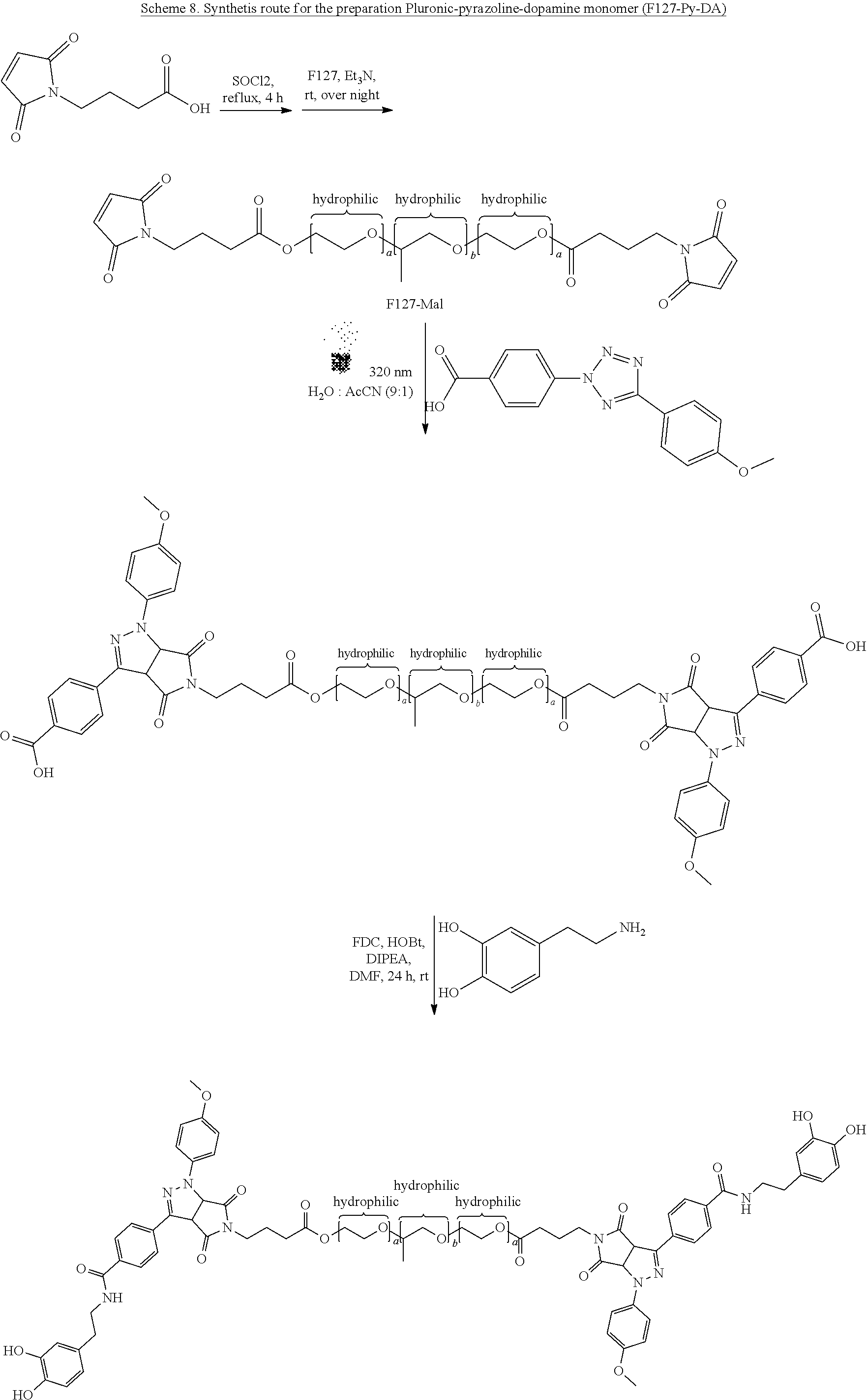

### Synthesis of Pluronic-Maleimide (F127-Mal)

First 4-maleimidobutiric acid (500 mg, 2.73 mmol) and thionyl chloride (3.9 mL, 53.8 mmol) were added into a heat-dried flask under a dry nitrogen atmosphere with dry benzene (20 mL).^{[6]} The reaction was maintained at 60° C. for 3 h. The excess thionyl chloride was removed under vacuum and the resulting yellow powder was used without further purification. F127 (2.5 g, 0.19 mmol) was added into a flask and heated to 120° C. for 4 h to remove water. After cool-down, 10 mL of anhydrous dichloromethane was added to dissolve the dried F127. The obtained 4-maleimidobutiric acyl chloride was dissolved in 10 mL of anhydrous dichloromethane and introduced into the F127 solution, followed by addition of triethylamine (107.8 μL, 0.78 mmol). The reaction mixture was stirred overnight under argon. The solvent was concentrated by rotary evaporation and the resulting product was subsequently dissolved in methanol:water (50:50), dialyzed against methanol:water (50:50) for 1 day, and then against water for another 2 days. The final product was obtained in the form of a white power after lyophilization of the dialyzed solution (1.3 g, yield: 56.7%). ¹H NMR (400 MHz, CDCl₃): δ (ppm) 1.09-1.17 (m, 195H), 1.90-1.96 (m, 4H), 2.33-2.38 (m, 4H), 3.35-3.44 (m, 67H), 3.48-3.60 (m, 136H), 3.61-3.71 (m, 830H), 4.21-4.24 (m, 4H), 6.71 (s, 4H).

### Synthesis of Pluronic-pyrazoline (F127-Py)

F127-Mal (750 mg, 0.058 mmol) and 4-(2-(4-methoxyphenyl)-2H-tetrazol-5-yl)benzoic acid^{[3]} (102.5 mg, 0.35 mmol) were dissolved in 75 mL acetonitrile, sonicated and stirred in a custom-built photoreactor^{[7]} under UV irradiation (320 nm, 36 W, Arimed B6, Cosmedico GmbH, Germany) for 6-8 h and monitored by fluorescence spectroscopy. The solvent was removed by rotary evaporation and the resulting product was subsequently dissolved in methanol:water (50:50), dialyzed against methanol:water (50:50) for 2 days, and then against water for another 2 days. The final product was obtained in the form of a yellow powder after lyophilization of the dialyzed solution (500 mg, yield: 63.2%). ¹H NMR (400 MHz, CDCl₃): δ (ppm) 0.99-1.26 (m, 195H), 1.86-1.95 (m, 4H), 2.26-2.34 (m, 4H), 3.27-3.44 (m, 70H), 3.45-3.59 (m, 133H), 3.57-3.72 (m, 813H), 3.89 (s, 6H), 4.39-4.34 (m, 4H), 4.91 (d, J=11.0 Hz, 2H), 5.14 (d, J=11.1 Hz, 2H), 7.91 (d, J=9.1 Hz, 4H), 7.98 (d, J=8.5 Hz, 4H), 8.10 (d, J=9.1 Hz, 4H), 8.30 (d, J=8.5 Hz, 4H).

### Synthesis of Pluronic-pyrazoline-dopamine (F127-Py-DA)

F127-Py (960 mg, 0.07 mmol) was dissolved in DMF (15 mL) followed by addition of NHS (20.5 mg, 0.18 mmol), DMAP (0.9 mg, 0.01 mmol), DCC (41.3 mg, 0.20 mmol) and dopamine hydrochloride (53.9 mg, 0.28 mmol). The reaction mixture was stirred protected from direct light and under inert atmosphere for 24 hours. The solvent was removed by rotary evaporation and the resulting product was subsequently dissolved in methanol:water (50:50), dialyzed against methanol:water (50:50) for 2 days, and then against water for another 2 days. The final product was obtained in the form of a yellow powder after lyophilization of the dialyzed solution (500 mg, yield: 51.8%). ¹H NMR (400 MHz, CDCl₃): δ (ppm) 1.019-1.13 (m, 195H), 1.81-1.95 (m, 4H), 2.26-2.34 (m, 4H), 2.55-2.62 (m, 4H), 3.29-3.42 (m, 70H), 3.46-3.56 (m, 140H), 3.56-3.70 (m, 836H), 3.88 (s, 6H), 4.90 (d, J=11.0 Hz, 2H), 5.15 (d, J=11.0 Hz, 2H), 6.49 (dd, J=8.0, 1.6 Hz, 2H), 6.63 (d, J=1.6 Hz, 2H), 6.70 (d, J=8.0 Hz, 2H), 6.90 (d, J=9.1 Hz, 4H), 7.50 (d, J=9.1 Hz, 4H), 7.87 (d, J=8.4 Hz, 4H), 8.04 (d, J=8.4 Hz, 4H).

### Synthesis of F127Py@PDA NPs (Similar to F127@PDA NPs, the Difference is the Monomer)

Trizma-base (22.5 mg) was dissolved in 2.5 mL Milli Q water and added to a mixture of ethanol and Milli-Q water (30 mL) and stirred for 30 min at room temperature. Dopamine hydrochloride (dissolved in 1 mL Milli Q water) and F127PyDA (dissolved in 1 mL ethanol) were mixed and sonicated before being added dropwise to the reaction mixture. The mixture was left to stir over night at room temperature resulting in a dark brown solution. The reaction mixture was washed with Milli-Q water using Vivaspin 20 (Satorius, UK) centrifugal concentrators (30 kDa MWCO) for 15 min at 4000 rpm until the supernatant was colorless. The obtained particles were diluted in Milli-Q water, frozen in liquid nitrogen and lyophilized to yield a dark brown powder.

TABLE 2
DLS and zeta potential (ζ) measurements of F127Py@PDA samples.
Errors are the standard deviation of the triplicate data. Sizes from
STEM images were determined from the mean of >100 measurements of
spherical particles, with the associated error being the standard deviation.

| | | | | | Hydro- | |
|---|---|---|---|---|---|---|
| | | | | | dynamic | |
| n(DA): | m(DA) | m(F127PyDA) | V(EtOH) | V(H₂O | diameter | |
| n(F127PyDA) | [mg] | [mg] | [mL] | [mL] | [nm] | PDI |
| 20:1 | 27.1 | 97.3 | 15.0 | 15.0 | 60.6 ± 1.1 | 0.109 |
| 30:1 | 44.48 | 18.4 | 15.0 | 15.0 | 66.8 ± 1.4 | 0.054 |
| 50:1 | 27.9 | 41.7 | 15.0 | 15.0 | 81.7 ± 1.9 | 0.063 |
| 75:1 | 28.0 | 27.8 | 15.0 | 15.0 | 100.9 ± 2.1 | 0.029 |
| 115:1 | 28.3 | 13.9 | 15.0 | 15.0 | 122.2 ± 0.6 | 0.032 |
| 115:1 | 28.3 | 13.9 | 7.5 | 22.5 | 84.8 ± 1.6 | 0.186 |
| 115:1 | 28.3 | 13.9 | 0 | 30 | 37.5 ± 2.2 | 0.074 |

^{a}not determined

### References for Part 2 of Examples

[1]L. Lebeau, P. Oudet, C. Mioskowski, Helv. Chim. Acta 1991, 74, 1697.
[2]M. Li, W. Jiang, Z. Chen, S. Suryaprakash, S. Lv, Z. Tang, X. Chen, K. W. Leong, Lab Chip 2017, 17, 635.
[3]C. Rodriguez-Emmenegger, C. M. Preuss, B. Yameen, O. Pop-Georgievski, M. Bachmann, J. O. Mueller, M. Bruns, A. S. Goldmann, M. Bastmeyer, C. Barner-Kowollik, Adv. Mater. 2013, 25, 6123.
[4]A. J. Sinclair, V. Del Amo, D. Philp, Org. Biomol. Chem. 2009, 7, 3308.
[5]L. D. Lavis, T. Y. Chao, R. T. Raines, ACS Chem. Biol. 2006, 1, 252.
[6]A. Herrmann, G. Mihov, G. W. M. Vandermeulen, H. A. Klok, K. Mullen, Tetrahedron 2003, 59, 3925.
[7]L. Stolzer, A. Vigovskaya, C. Barner-Kowollik, L. Fruk, Chemistry 2015, 21, 14309.

### Examples—Part 3

1. EGFP-Encoding pDNA Delivery in HEK-293 Cells with Poly-L-Arginine and Poly-L-histidine functionalized F127@PDAF127PDA NPs

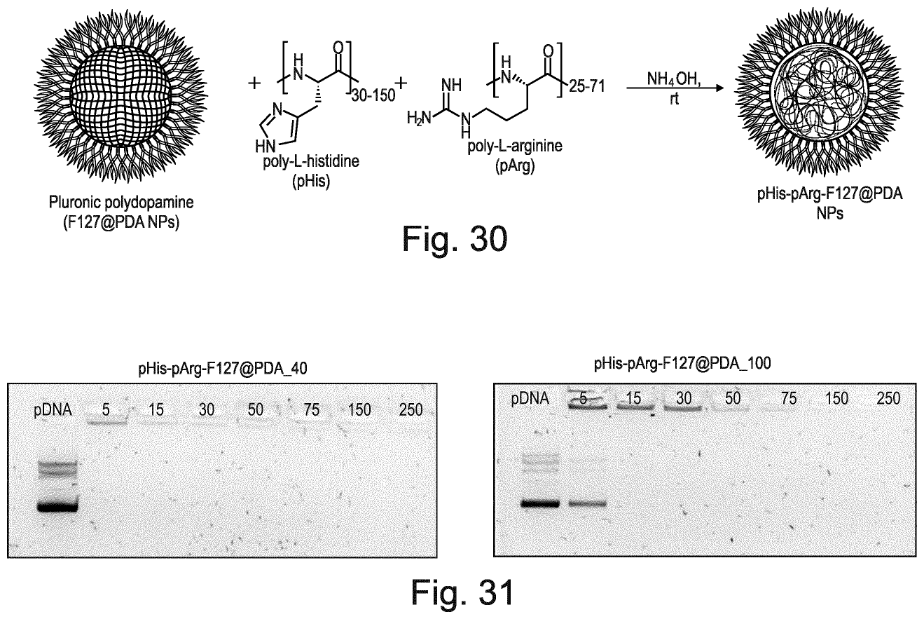

Pluronic polydopamine NPs were validate for the delivery of genetic material (EGFP encoding pDNA). Since F127@PDA NPs have a negative surface potential, they were modified with a mixture of poly-L-arginine (pArg, M_{w}=5-15 kDa) and poly-L-histidine (pHis, M_{w}=5-25 kDa) under mild basic conditions (10 mM NH₄OH) for 24 h, resulting in the formation of pHis-pArg-F127@PDAF127PDA_40 and pHis-pArg-F127@PDAF127PDA_100 (FIG. 30). The resulting NPs showed an increase in hydrodynamic size and positive zeta-potential allowing for the immobilisation of pDNA (Table 1). First the ability of the NPs to immobilise pDNA was determined with agarose gel electrophoresis (FIG. 31). The pDNA complexes with the pArg-pHis modified F127@PDAF127PDA NPs were prepared in different weight ratios of NP to pDNA. pHis-pArg-F127@PDAF127PDA_40 NPs were able to fully bind the pDNA bellow weigh ratio 5 (WR-5), while pHis-pArg-F127@PDAF127PDA_100 NPs this was achieved bellow weight ratio 15.

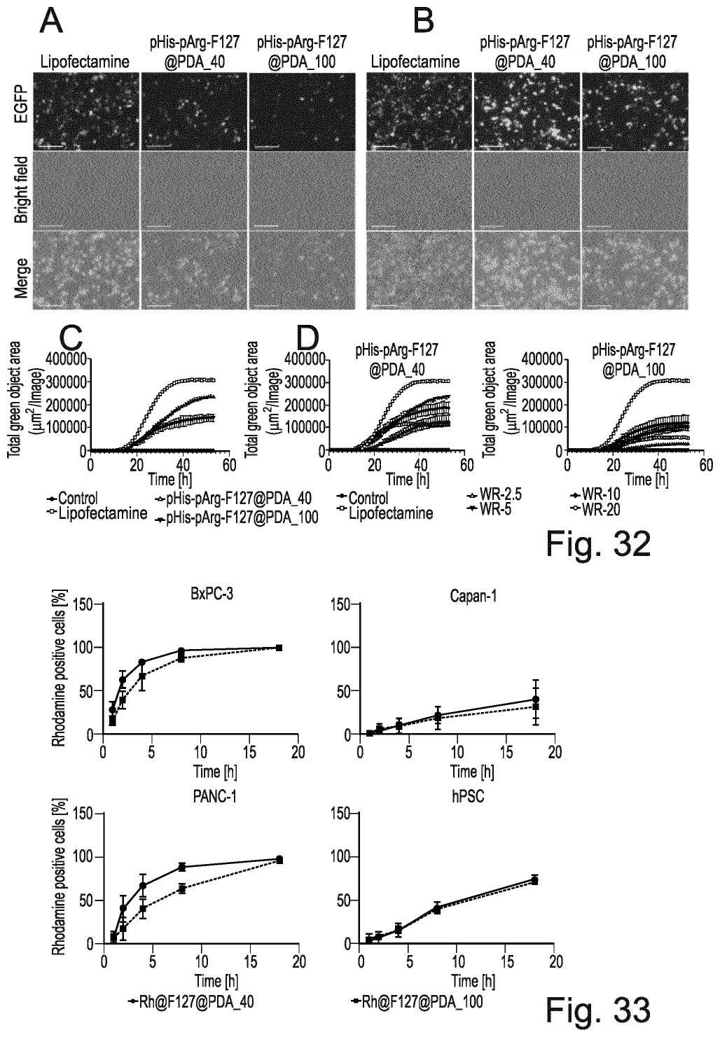

Following successful immobilisation of the EGFP-encoding pDNA, their transfection efficacy was evaluated in human embryonic kidney cells (HEK-293). Live-cell imaging (IncuCyte) was conducted to monitor the EGFP expression over 48 h. Different weight ratios of NPs to pDNA (WR-2.5, WR-5, WR-10 and WR-20) were evaluated (FIG. 32). Lipofectamine was used as a positive control. Both 40 and 100 nm pHis-pArg functionalized F127@PDAF127PDA NPs resulted in EGFP transfection, with a significant increase with longer (48 h) treatment. The 40 nm NPs resulted in greater transfection efficacy compared to the 100 nm NPs. An increased efficacy was also observed with increased ratio of NP to pDNA until WR-5 for 40 nm NPs and WR-10 for 100 nm NPs, after which a decrease in the EGFP expression was observed. The 40 nm NPs resulted in a 51.3% transfection efficacy compared to the Lipofectamine positive control after 24 h and 76.8% efficacy after 48 h, while the 100 nm NPs showed 40.4% transfection efficacy after 24 h and 46.2% efficacy after 48 h.

TABLE 1
Hydrodynamic diameter (Z-average), polydispersity index (PDI) and zeta potential
(ζ) of F127@PDAF127PDA NPs before and after pHis-pArg functionalization.

| | Z-average | | ζ |
|---|---|---|---|
| | (nm) | PDI | (mV) |
| F127@PDAF127PDA_40 | 63.82 ± 3.7 | 0.069 ± 0.024 | −21.5 ± 2.1 |
| pHis-pArg- | 98.26 ± 2.81 | 0.165 ± 0.061 | +44.7 ± 0.6 |
| F127@PDAF127PDA_40 | | | |
| F127@PDAF127PDA_100 | 117.4 ± 2.2 | 0.016 ± 0.018 | −18.9 ± 1.7 |
| pHis-pArg- | 181.2 ± 3.9 | 0.259 ± 0.29 | +41.5 ± 0.9 |
| F127@PDAF127PDA_100 | | | |

### Experimental Procedures

### Poly-L-Histidine and Poly-L-Arginine Functionalization of F127@PDAF127PDA NPs

The Pluronic-PDA NPs (5 mg) were dissolved in 10 mM NH₄OH (5 mL). Poly-L-arginine (pArg, M_{w}=5-15 kDa, Sigma) was dissolved in Mili-Q water (3 mg in 0.5 mL) and poly-L-histidine (pHis, M_{w}=5-25 kDa, Sigma) was resuspended in DMSO (7 mg in 0.5 mL) and added dropwise to the NP solution. The mixture was stirred over night and the NPs were washed in several centrifugation steps and stored at 4° C. as a 5 mg/mL solution. DLS and zeta potential measurements were recorded using a Zetasizer Nano ZS instrument (Malvern Panalytical, UK).

### Gel Electrophoresis

To evaluate the pDNA immobilisation to the NPs gel electrophoresis with 1% agarose gels in 1× Tris-acetate-EDTA (TAE) buffer in a Bio-Rad Sub-Cell electrophoresis system were conducted. SYBR Safe stain (4 μL) were added to the hot agarose solution (60 mL) prior to casting. For immobilisation studies, various volumes (0.3, 0.9, 1.8, 3.0, 4.5, 9.0 and 15 μL) of 5.00 mg/mL NP stock solutions were prepared and diluted with Milli-Q water to a total volume of 15 μL. The samples were incubated with 300 ng of pDNA for 15 min. Prior to loading, 2 μL of 10× Orange-G loading dye in glycerol was added to each sample. The gel was electrophoresed at 80 V for 40 min and imaged in a Syngene G:BOX Gel Documentation System.

### IncuCyte Transfection Experiment

Human embryonic kidney cells (HEK-293) were purchased from American Type Culture Collection (ATCC). The cells were grown in DMEM (Sigma, UK) supplemented containing 10% FBS and 0.5% pen-strep and cultured in a humidified environment at 37° C. with 5% CO₂. The cells were seeded in black 96-well plates (Corning, #3904) at a concentration of 18000 cells/well in 100 μl of complete growth medium and incubated at 37° C., 5% CO₂ for 24 h. Varying amounts of 5.00 mg/mL NPs stock solution (0.625, 1.25, 2.5 and 5 μL) were diluted with Milli-Q water to a final volume of 5 μL and incubated with 250 ng pDNA. After 15 min incubation DMEM (10% FBS) 50 μL was added to each sample and to the 96-well plates. The concentrations correspond to a NP to pDNA weight ratio WR-2WR2.5, WR-5, WR-10WR5, WR10 and WR-20WR20. The plates were then inserted into the IncuCyte®S3 Live Cell Analysis System (Sartorius) for real-time imaging. Treated plates were imaged every hour for 48 h under cell culture conditions with a 20× objective using the green and brightfield channel. The mean fluorescence intensity was taken from 4 random fields of view per well and calculated with the IncuCyte S3 v2017A software.

### 2. Time-Dependent Uptake of Rhodamine@F127@PDAF127PDA NPs in 2D Pancreatic Cancer Cells

Rhodamine@F127@PDA (Rh@F127@PDA)F127PDA NPs were used to study their time-dependent uptake in different pancreatic cancer cells (BxPC-3, Capan-1 and PANC-1), as well as human pancreatic stellate cells (hPSC). Flow cytometry was used to determine the percentage of Rhodamine-positive cells for Rh@F127@PDAF127PDA_40 and Rh@F127@PDAF127PDA_100 compared to untreated cells (FIG. 33). As shown in FIG. 33, both 40 and 100 nm NPs showed above 95% uptake after 18 h in BxPC-3 and PANC-1 cells, while 50% Capan-1 cells and 70% hPSC were Rhodamine positive after 18 h. The 100 nm NPs showed slower uptake for BxPC-3 and PANC-1 cells, while there were no significant differences in the uptake kinetic of the two studied sizes in Capan-1 and hPSC cells.

### Experimental Procedure

The cells were seeded in 6-well plates at a density of 2×10⁵ cells per well and cultured for 24 h. The cells were treated for different time points (1, 2, 4, 8 and 18 h) with Rh@RhodamineF127@PDAF127PDA NPs 40 and 100 nm in size at a concentration of 30 μgmL⁻¹. After the treatment, cells were washed with PBS, detached with 0.25 mL TrypLE (Thermo Fischer, UK) and resuspended with FACS buffer (PBS with 4% FBS). The cell suspensions were centrifuged for 15 min at 300 g. 1 mL of FACS buffer (PBS with 4% FBS) was added to the cells and containing 10 μgmL⁻¹ DAPI. The cells were kept at 4° C. until flow cytometry analysis. Flow cytometry was carried out on a CyAn ADP flow cytometer (Agilent) using 355 and 488 lasers. 100 000 events were acquired for each sample. FlowJo software (version 10.2) was used for data analysis. Briefly, the live single-cell population was gated in a plot of FSC vs. SSC after excluding cell debris and doublets a histogram from the PE channel for the single-cell population was obtained and analysed.

### 3. Pulsed Efficacy Studies of Gemcitabine and SN38 Loaded F127(a)PDAF127PDA NPs in 2D Pancreatic Cancer Cells

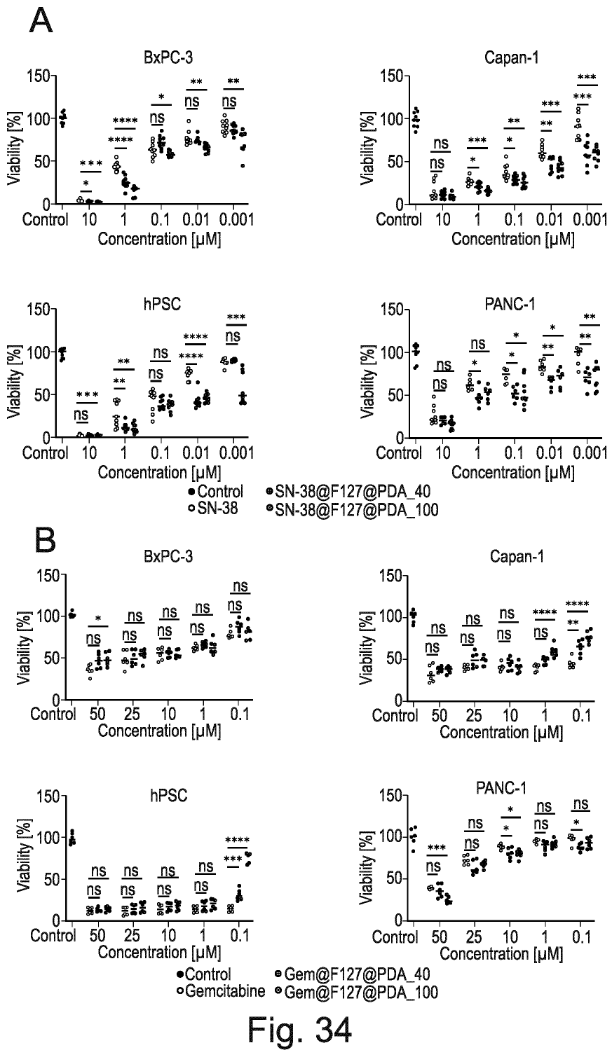

In order to validate whether prolonged release of the drug can be achieved with F127@PDAF127PDA NPs, pulsed release studies were conducted in vitro with BxPC-3, Capan-1, PANC-1 and hPSC cells. The cells were treated with Gemcitabine and SN38 formulations in F127@PDAF127PDA NPs for 18 h. After the treatment cells were washed and their recovery was measured after 72 h using MTS endpoint assay. Both NP uptake and drug sensitivity play an important role in the efficacy of the formulations. As noted in FIG. 34A both 40 and 100 nm SN38@F127@PDAF127PDA formulation show a significant difference in efficacy compared to the free SN38 drug for most of the tested concentrations. Indicating that the formulations show prolonged release of SN38. However, the 40 and 100 nm Gem@F127@PDAF127PDA formulations, show no significant differences or even lower efficacy compared to free Gemcitabine (FIG. 34B).

### Experimental Procedure

Cells were seeded into 96-well plates at concentration of 4×10³ cells per well, in 100 μL of complete growth medium and incubated at 37° C., 5% CO₂ for 24 h. After overnight incubation, the cells were treated with SN38 or Gemcitabine (Gem) and the same concentration of the drug in the SN38@F127@PDAF127PDA or Gem@F127@PDAF127PDA formulation. After treatment for 18 h, the cells were washed with 1×PBS two times and fresh media was added (100 μL). After 72 h the cell viability was determined using MTS assay as described previously.

### 4. Validation of SN38 Loaded F127(a)PDAF127PDA NPs in 3D Pancreatic Spheroids

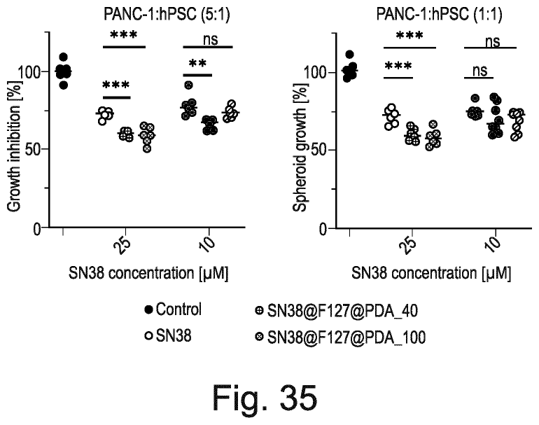

In vitro efficacy of SN38-loaded (SN38@F127PDA) F127@PDA NPs were validate in multicellular pancreatic cancer (PANC-1) and human pancreatic stellate cells (hPSC) spheroids. Two different models with a ratio of PANC-1:hPSC (5:1 and 1:1) were used to study the drug efficacy. The spheroids were treated with the drug formulations for 18 h pulse, after which they were washed, and the recovery was monitored for 96 h (FIG. 35). The efficacy of the drug loaded NPs and the free drugs were determined by the growth inhibition of the spheroids measured with IncuCyte live-cell imaging and the growth inhibition was normalized to untreated spheres. A statistically significant difference was only observed for treatment with 25 μM of SN38 formulation within the NPs, while lower concentrations did not result in any significant differences between the formulations.

### Experimental Procedure for the Drug Screening

Ultra-low attachment 96-well plates were used to seed the spheroids. Cells were seeded at a total density of 5×10³ cells per well containing PANC-1:hPSC at a 5:1 and 1:1 ratio was added to each well in 100 μL of complete growth media. To each well 100 μL of 5% Matrigel (#354234, Corning) in DMEM containing 10% FBS and 0.5% PenStrep was added so that the final Matrigel concentration in each well was 2.5%. The plates were centrifuged at 2000 rpm for 15 min. The plates were incubated at 37° C., 5% CO₂ for 5 days for the spheroids to form. The spheroids were treated for 18 h with SN38 and the same concentration of SN38@F127@PDAF127PDA NPs (25, 10, 1 and 0.1 μM). The plates were inserted into the IncuCyte®S3 Live Cell Analysis System (Sartorius) for real-time imaging. After 18 h the cells were washed with 1×PBS two times and 100 μL of complete growth media was added to each well. The plates were imaged every 3 h for 96 h under cell culture conditions with 4× objective using the brightfield channel. The largest brightfield object area was used to determine the size of the spheroids. The growth inhibition was calculated according to the following equation:

$$Growth inhibition ( \% ) = 100 \times [ ( Spheroid size after 96 h recovery / Spheroid size before treatment ) / \\ ( Spheroid size of untreated control after 96 h recovery / \\ Spheroid size of untreated control before treatment )$$

### Numbered Paragraphs

The following numbered paragraphs define particular aspects and embodiments of the invention.
1. Polydopamine co-polymer nanoparticles comprising polydopamine having a co-polymer of poly(ethylene oxide) and poly(propylene oxide) covalently bound thereto.
2. Polydopamine co-polymer nanoparticles according to paragraph 1, wherein the polydopamine co-polymer nanoparticles have a particle size of less than or equal to 140 nm.
3. Polydopamine co-polymer nanoparticles according to paragraph 1 or paragraph 2, wherein the polydopamine co-polymer nanoparticles have a particle size of 30 to 140 nm.
4. Polydopamine co-polymer nanoparticles according to any one of the preceding paragraphs, wherein the polydopamine co-polymer nanoparticles have a particle size of 40 to 100 nm.
5. Polydopamine co-polymer nanoparticles according to any one of the preceding paragraphs, wherein the polydopamine co-polymer nanoparticles have a particle size of 40 to 60 nm.
6. Polydopamine co-polymer nanoparticles according to any one of the preceding paragraphs, wherein the co-polymer of poly(ethylene oxide) and poly(propylene oxide) is a block co-polymer.
7. Polydopamine co-polymer nanoparticles according to any one of the preceding paragraphs, wherein the co-polymer of poly(ethylene oxide) and poly(propylene oxide) is a tri-block co-polymer having a central block of poly(propylene oxide) flanked on each side by blocks of poly(ethylene oxide).
8. Polydopamine co-polymer nanoparticles according to any one of the preceding paragraphs, wherein the co-polymer has the formula:

-[poly(ethylene oxide)]-[poly(propylene oxide)]-[poly(ethylene oxide)]-

or

—X₁-[poly(ethylene oxide)]-[poly(propylene oxide)]-[poly(ethylene oxide)]-X₂—

wherein: X₁ and X₂ are each independently:
a linker group that connects the co-polymer to the polydopamine; or
a detectable moiety (e.g. a fluorophore) that connects the co-polymer to the polydopamine.
   9. Polydopamine co-polymer nanoparticles according to any one of the preceding paragraphs, wherein the co-polymer has the formula:

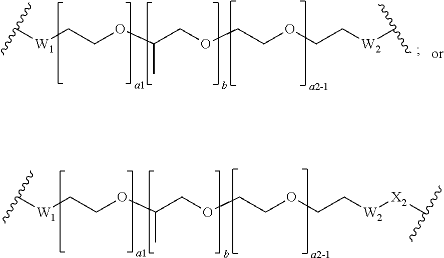

wherein:
W₁ and W₂ are selected from O or NH; or
W₁ and W₂ are selected from:

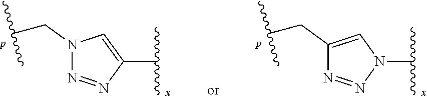 [SMILES: *Cn1cc(*)nn1 ; *Cc1cn(*)nn1]

wherein
p indicates the bond to the co-polymer;
x indicates the bond to the X₁ or X₂ group, if present, or the polydopamine;
a1 is an integer between 50 and 150;
a2 is an integer between 50 and 150;
b is an integer between 20 and 80; and
X₁ and X₂ are as defined in paragraph 8.
   10. Polydopamine co-polymer nanoparticles according to paragraph 9, wherein:
W₁ and W₂ are as defined in paragraph 9;
a1 is an integer between 75 and 125;
a2 is an integer between 75 and 125; and
b is an integer between 25 and 75.
   11. Polydopamine co-polymer nanoparticles according to paragraph 9 or paragraph 10, wherein:
W₁ and W₂ are as defined in paragraph 9;
a1 is an integer between 90 and 110;
a2 is an integer between 90 and 110; and
b is an integer between 45 and 65.
   12. Polydopamine co-polymer nanoparticles according to paragraph 9, 10 or 11, wherein:
W₁ and W₂ are as defined in paragraph 9;
a1 and a2 are the same.
   13. Polydopamine co-polymer nanoparticles according to any one of paragraphs 9 to 12, wherein:
W₁ and W₂ are as defined in paragraph 9;
a1 is 101;
a2 is 101; and
b is 56 (i.e. Pluronic F127).
   14. Polydopamine co-polymer nanoparticles according to any one of paragraphs 9 to 13, wherein:
W₁ and W₂ are selected from O or NH; and
X₁ and X₂ are each independently:
   (i) a linker of the formula:

—C(═O)—[CH₂]ₙ—C(═O)—
wherein:
n is an integer from 1 to 10; or
(ii) a fluorophore.
   15. Polydopamine co-polymer nanoparticles according to any one of paragraphs 9 to 13, wherein either:
A) W₁ and W₂ are selected from O or NH; and
   X₁ and X₂ are each independently:
      (i) a linker of the formula:

—C(═O)—[CH₂]ₙ—C(═O)—
wherein n is an integer from 1 to 10; or
(ii) a fluorophore of the formula:

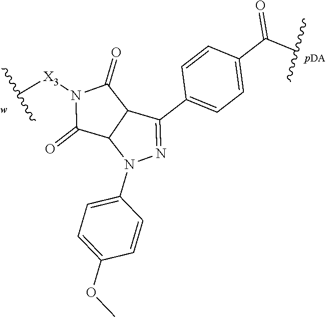

wherein:
   w indicates the bond to the W₁ or W₂ group;
   pDA indicates the bond to the polydopamine;
   X₃ is selected from:
   (i) —C(═O)—[CH₂]ₙ₁—. wherein n1 is an integer from 2 to 10;
   (ii) —[CH₂]ₙ₂—, wherein n2 is an integer from 2 to 10;
   (iii) —C(═O)—CH₂CH₂—[OCH₂CH₂]ₙ₃—, n3 is an integer from 2 to 10;
   (iii) a fluorophore of the formula:

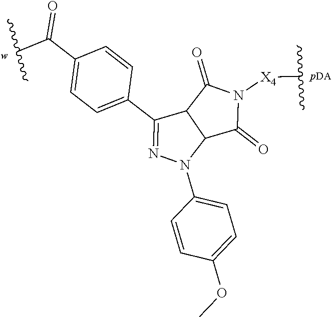

wherein:
   w indicates the bond to the W₁ or W₂ group;
   pDA indicates the bond to the polydopamine;
   X₄ is selected from:
   (i) —[CH₂]ₙ₄—C(═O)—, wherein n4 is an integer from 2 to 10;
   (ii) —[CH₂]ₙ₅—, wherein n5 is an integer from 2 to 10;
   (iii) —[CH₂CH₂O]ₙ₆—CH₂CH₂—C(═O)—, wherein n6 is an integer from 2 to 10; or
B) W₁ and W₂ are each a group of the formula:

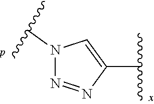 [SMILES: *c1cn(*)nn1]

wherein
p indicates the bond to the co-polymer;
x indicates the bond to the X₁ or X₂ group;
X₁ and X₂ are each selected from:
(i) —CH₂—
(ii) —CH₂CH₂—[OCH₂CH₂]ₙ₇—, wherein n7 is an integer from 2 to 10;
(iii) —CH₂—C(═O)—
(iv) —CH₂CH₂—[OCH₂CH₂]ₙ₈—NH—C(═O)—, wherein n8 is an integer from 2 to 10;
(v) —CH₂CH₂—[OCH₂CH₂]ₙ₉—O—C(═O)—, wherein n9 is an integer from 2 to 10; and
e) W₁ and W₂ are each a group of the formula:

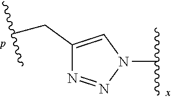 [SMILES: *Cc1cn(*)nn1]

wherein
   p indicates the bond to the co-polymer;
   x indicates the bond to the X₁ or X₂ group;
   X₁ and X₂ are each selected from:
   (i) —CH₂CH₂—[OCH₂CH₂]ₙ10, wherein n10 is an integer from 2 to 10;
   (ii) —CH₂CH₂—[OCH₂CH₂]ₙ₁₁, wherein n11 is an integer from 2 to 10;
   (iii) —[CH₂CH₂O]ₙ₁₂—C(═O)—CH₂CH₂—C(═O), wherein n12 is an integer from 2 to 10; and
   (vi) —[CH₂CH₂O]ₙ₁₃—CH₂CH₂—NH—C(═O)—, wherein n13 is an integer from 2 to 10;
   (vii) —CH₂CH₂—[OCH₂CH₂]ₙ₁₄—O—C(═O), wherein n14 is an integer from 2 to 10;
   (viii) —[CH₂CH₂O]ₙ₁₅—C(═O)—CH₂CH₂—C(═O), wherein n15 is an integer from 2 to 10;
      16. Polydopamine co-polymer nanoparticles according to any one of paragraphs 9 to 15, wherein W₁ and W₂ are selected from O or NH; and
X₁ and X₂ are each independently:
   (i) a linker of the formula:

—C(═O)—[C H₂]ₙ—C(═O)—
wherein n is an integer from 2 to 8; or
(ii) a fluorophore of the formula:

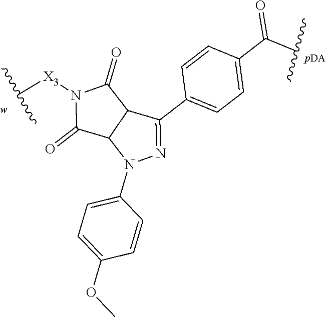

wherein:
   w indicates the bond to the W₁ or W₂ group;
   pDA indicates the bond to the polydopamine;
   X₃ is selected from:
      (i) —C(═O)—[CH₂]ₙ₁—. wherein n1 is an integer from 2 to 8;
      (ii) —[CH₂]ₙ₂—, wherein n2 is an integer from 2 to 8;
      (iii) —C(═O)—CH₂CH₂—[OCH₂CH₂]ₙ₃—, n3 is an integer from 2 to 8;
(iii) a fluorophore of the formula:

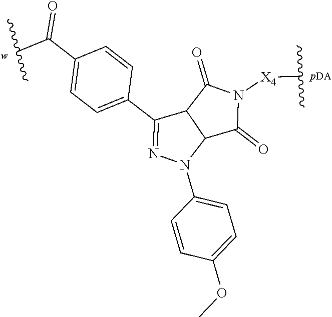

wherein:
   w indicates the bond to the W₁ or W₂ group;
   pDA indicates the bond to the polydopamine;
   X₄ is selected from:
   (i) —[CH₂]ₙ₄—C(═O). wherein n4 is an integer from 2 to 8;
   (ii) —[CH₂]ₙ₅—, wherein n5 is an integer from 2 to 8;
   (iii) —CH₂CH₂—[CH₂CH₂O]ₙ₆—C(═O)—, wherein n6 is an integer from 2 to 8.
      17. Polydopamine co-polymer nanoparticles according to any one of paragraphs 9 to 16, wherein W₁ and W₂ are selected from O or NH; and
X₁ and X₂ are each independently:
   (i) a linker of the formula:

—C(═O)—[C H₂]ₙ—C(═O)—
wherein n is an integer from 2 to 8; or
(ii) a fluorophore of the formula:

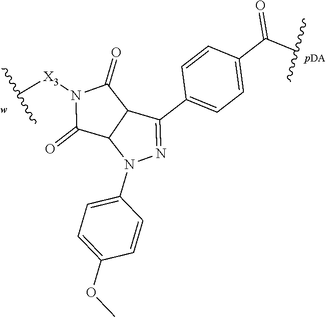

wherein:
   w indicates the bond to the W₁ or W₂ group;
   pDA indicates the bond to the polydopamine;
   X₃ is —C(═O)—[CH₂]ₙ₁—. wherein n1 is an integer from 2 to 8.
      18. Polydopamine co-polymer nanoparticles according to any one of paragraphs 9 to 17, wherein W₁ and W₂ are selected from O or NH, and X₁ and X₂ are each independently a linker of the formula:

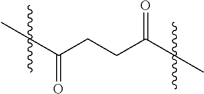 [SMILES: *C(=O)CCC(*)=O]

or a fluorophore of the formula:

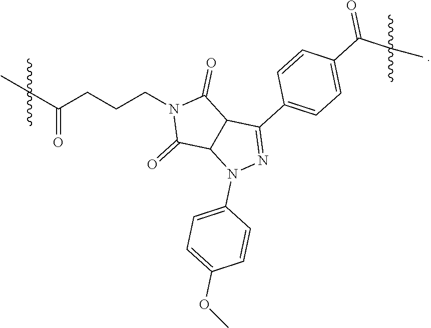 [SMILES: *C(=O)CCCN1C(=O)C2C(c3ccc(C(*)=O)cc3)=NN(c3ccc(OC)cc3)C2C1=O]

19. Polydopamine co-polymer nanoparticles according to any one of paragraphs 9 to 18, wherein W₁ and W₂ are each O.
20. Polydopamine co-polymer nanoparticles according to any one of the preceding paragraphs, wherein the polydopamine co-polymer nanoparticles further comprise a functional moiety covalently attached or adsorbed to the nanoparticle.
21. Polydopamine co-polymer nanoparticles according to paragraph 20, wherein the functional moiety covalently attached or adsorbed to the nanoparticle is a moiety selected from the group consisting of a pharmacologically active agent (e.g. a drug or biologic), a targeting ligand (e.g. a receptor ligand, antibody or nanobody), or an imaging agent (e.g. a detectable moiety, such as a fluorophore, magnetic particles and/or radionuclides).
22. A pharmaceutical composition comprising polydopamine co-polymer nanoparticles according to any one of paragraphs 1 to 19, a pharmacologically active agent (e.g. a drug or biologic) and a pharmaceutically acceptable excipient.
23. A pharmaceutical composition comprising polydopamine co-polymer nanoparticles according to paragraph 22 and a pharmacologically active agent (e.g. a drug or biologic) or an imaging agent (e.g. a detectable moiety, such as a fluorophore, magnetic particles and/or radionuclides), dispersed in an aqueous vehicle.
24. A process for preparing polydopamine co-polymer nanoparticles according to any one of paragraphs 1 to 20, the process comprising polymerising a catecholamine (e.g. dopamine) or DOPAC monomer with a monomer of a catecholamine (e.g. dopamine) or DOPAC, that is covalently bound to a co-polymer of poly(ethylene oxide) and poly(propylene oxide) to form polydopamine co-polymer nanoparticles having a co-polymer of poly(ethylene oxide) and poly(propylene oxide) covalently bound thereto.
25. A process for preparing polydopamine co-polymer nanoparticles according to paragraph 24, wherein, in the monomer of a catecholamine (e.g. dopamine) or DOPAC that is covalently bound to a co-polymer of poly(ethylene oxide) and poly(propylene oxide), the co-polymer of poly(ethylene oxide) and poly(propylene oxide) is a tri-block co-polymer having a central block of poly(propylene oxide) flanked on each side by blocks of poly(ethylene oxide).
26. A process for preparing polydopamine co-polymer nanoparticles according to paragraph 24 or paragraph 25, wherein, in the monomer of a catecholamine (e.g. dopamine) or DOPAC that is covalently bound to a co-polymer of poly(ethylene oxide) and poly(propylene oxide), the co-polymer of poly(ethylene oxide) and poly(propylene oxide) has the formula:

-[poly(ethylene oxide)]-[poly(propylene oxide)]-[poly(ethylene oxide)]-

or

—X₁-[poly(ethylene oxide)]-[poly(propylene oxide)]-[poly(ethylene oxide)]-X₂—
wherein:
X₁ and X₂ are each independently:
   a linker group that connects the co-polymer to the polydopamine; or
   a detectable moiety (e.g. a fluorophore) that connects the co-polymer to the polydopamine.
      27. A process for preparing polydopamine co-polymer nanoparticles according to any one of paragraphs 24 to 26, wherein the monomer of a catecholamine (e.g. dopamine) or DOPAC that is covalently bound to a co-polymer of poly(ethylene oxide) and poly(propylene oxide) has the formula:

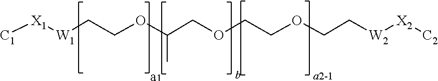

wherein:
W₁ and W₂ are selected from O or NH; or
W₁ and W₂ are selected from:

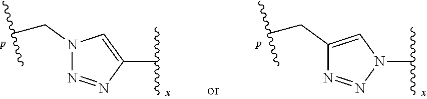 [SMILES: *Cn1cc(*)nn1 ; *Cc1cn(*)nn1]

wherein:
p indicates the bond to the co-polymer;
x indicates the bond to the X₁ or X₂ group,
a1 is an integer between 50 and 150;
a2 is an integer between 50 and 150;
b is an integer between 20 and 80; and
X₁ and X₂ are each independently:
   a linker group; or
   a detectable moiety (e.g. a fluorophore); and
C₁ and C₂ are a catecholamine or DOPAC.
   28. A process for preparing polydopamine co-polymer nanoparticles according to paragraph 27, wherein:
A) W₁ and W₂ are selected from O or NH; and either
   i) X₁ and X₂ are each a group of the formula:

—C(═O)—[C H₂]ₙ—C(═O)—
wherein n is an integer from 1 to 10; or
ii) X₁ and X₂ are each a group of the formula:

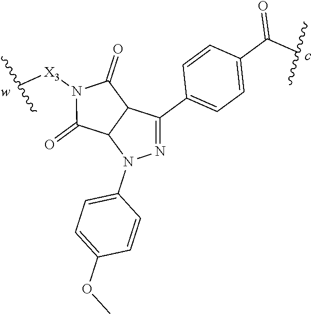

wherein:
w indicates the bond to the W₁ or W₂ group;
c indicates the bond to the C₁ or C₂ group;
X₃ is selected from:
   (i) —C(═O)—[CH₂]ₙ₁—. wherein n1 is an integer from 2 to 10;
   (ii) —[CH₂]ₙ₂—, wherein n2 is an integer from 2 to 10;
   (iii) —C(═O)—CH₂CH₂—[OCH₂CH₂]ₙ₃—, n3 is an integer from 2 to 10;
and
C₁ and C₂ are a group of a formula selected from:

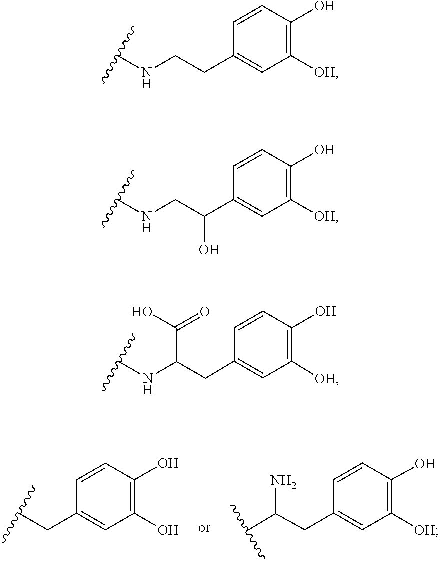 [SMILES: *NCCc1ccc(O)c(O)c1 ; *NCC(O)c1ccc(O)c(O)c1 ; *NC(Cc1ccc(O)c(O)c1)C(=O)O ; *Cc1ccc(O)c(O)c1 ; *C(N)Cc1ccc(O)c(O)c1]

B) W₁ and W₂ are selected from O or NH; and
   X₁ and X₂ are each a group of the formula:

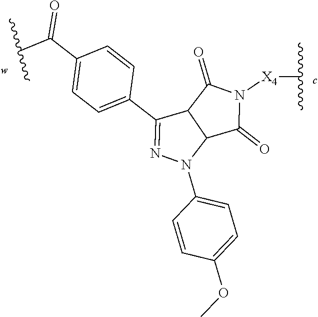

w indicates the bond to the W₁ or W₂ group;
c indicates the bond to the C₁ or C₂ group;
either
i) X₄ is selected from:
   (i) —[CH₂]ₙ₄—C(═O). wherein n4 is an integer from 2 to 10;
   (ii) —[CH₂]ₙ₅—, wherein n5 is an integer from 2 to 10;
   (iii) —[CH₂CH₂O]ₙ₆—CH₂CH₂—C(═O)—, wherein n6 is an integer from 2 to 10; and
   C₁ and C₂ are each a group selected from:

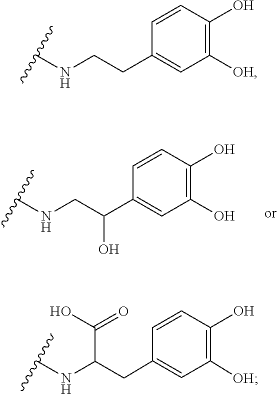 [SMILES: *NCCc1ccc(O)c(O)c1 ; *NCC(O)c1ccc(O)c(O)c1 ; *NC(Cc1ccc(O)c(O)c1)C(=O)O]

or
   ii) X₄ is selected from:
      (i) —[CH₂]ₙ₄—C(═O). wherein n4 is an integer from 2 to 10;
      (ii) —[CH₂]ₙ₅—C(═O), wherein n5 is an integer from 2 to 10;
      (iii) —[CH₂CH₂O]ₙ₆—CH₂CH₂—C(═O)—, wherein n6 is an integer from 2 to 10; and
   C₁ and C₂ are each a group of the formula:

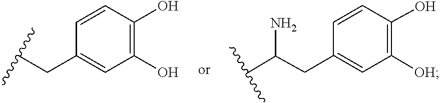 [SMILES: *Cc1ccc(O)c(O)c1 ; *C(N)Cc1ccc(O)c(O)c1]

C) W₁ and W₂ are each a group of the formula:

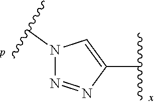 [SMILES: *c1cn(*)nn1]

wherein
p indicates the bond to the co-polymer;
x indicates the bond to the X₁ or X₂ group;
wherein either
i) X₁ and X₂ are each selected from:
(i) —CH₂—
(ii) —CH₂CH₂—[OCH₂CH₂]ₙ₇—, wherein n7 is an integer from 2 to 10; and
C₁ and C₂ are a group of a formula selected from:

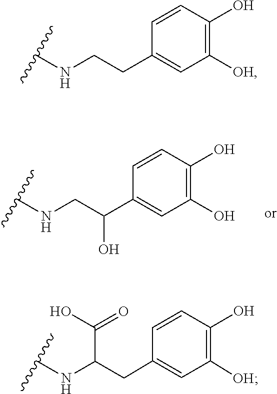 [SMILES: *NCCc1ccc(O)c(O)c1 ; *NCC(O)c1ccc(O)c(O)c1 ; *NC(Cc1ccc(O)c(O)c1)C(=O)O]

or
ii) X₁ and X₂ are each selected from:
   (i) —CH₂—C(═O)—;
   (ii) —CH₂CH₂—[OCH₂CH₂]ₙ₈NH—C(═O)—, wherein n8 is an integer from 2 to 10;
   (iii) —CH₂CH₂—[OCH₂CH₂]ₙ₉—O—C(═O)—, wherein n9 is an integer from 2 to 10; and
   C₁ and C₂ are a group of the formula:

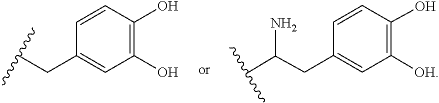 [SMILES: *Cc1ccc(O)c(O)c1 ; *C(N)Cc1ccc(O)c(O)c1]

D) W₁ and W₂ are each a group of the formula:

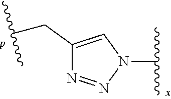 [SMILES: *Cc1cn(*)nn1]

wherein
   p indicates the bond to the co-polymer;
   x indicates the bond to the X₁ or X₂ group;
wherein either
i) X₁ and X₂ are each selected from:
   (i) —CH₂CH₂—[OCH₂CH₂]ₙ₁₀, wherein n10 is an integer from 2 to 10;
   (ii) —CH₂CH₂—[OCH₂CH₂]ₙ₁₁ wherein n11 is an integer from 2 to 10;
   (iii) —[CH₂CH₂O]ₙ₁₂—C(═O)—CH₂CH₂—C(═O), wherein n12 is an integer from 2 to 10; and
   C₁ and C₂ are a group of the formula:

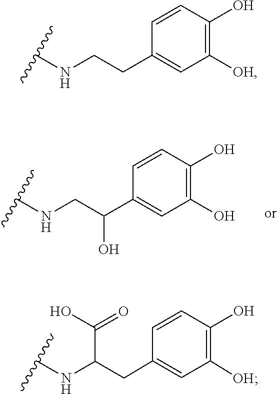 [SMILES: *NCCc1ccc(O)c(O)c1 ; *NCC(O)c1ccc(O)c(O)c1 ; *NC(Cc1ccc(O)c(O)c1)C(=O)O]

ii) X₁ and X₂ are each a group selected from:
   (i) —[CH₂CH₂O]ₙ₁₃—CH₂CH₂—NH—C(═O)—, wherein n13 is an integer from 2 to 10;
   (ii) —CH₂CH₂—[OCH₂CH₂]ₙ₁₄—O—C(═O), wherein n14 is an integer from 2 to 10;
   (iii) —[CH₂CH₂O]ₙ₁₅—C(═O)—CH₂CH₂—C(═O), wherein n15 is an integer from 2 to 10;
   and
   C₁ and C₂ are a group of the formula:

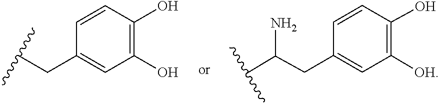 [SMILES: *Cc1ccc(O)c(O)c1 ; *C(N)Cc1ccc(O)c(O)c1]

29. A process for preparing polydopamine co-polymer nanoparticles according to paragraph 28, wherein W₁ and W₂ are selected from O or NH; and
either
i) X₁ and X₂ are each a group of the formula:

—C(═O)—[C H₂]ₙ—C(═O)—
wherein n is an integer from 1 to 10; or
ii) X₁ and X₂ are each a group of the formula:

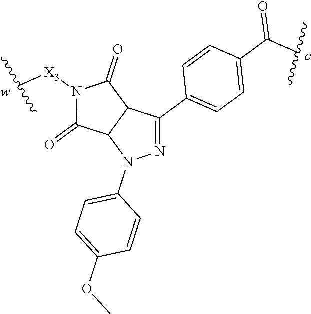

wherein:
w indicates the bond to the W₁ or W₂ group;
c indicates the bond to the C₁ or C₂ group; and
X₃ is selected from:
   (i) —C(═O)—[CH₂]ₙ₁—. wherein n1 is an integer from 2 to 10;
   (ii) —[CH₂]ₙ₂—, wherein n2 is an integer from 2 to 10; and
   (iii) —C(═O)—CH₂CH₂—[OCH₂CH₂]ₙ₃—, n3 is an integer from 2 to 10;
and C₁ and C₂ are each a group:

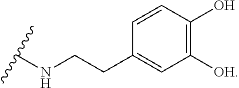 [SMILES: *NCCc1ccc(O)c(O)c1]

30. A process for preparing polydopamine co-polymer nanoparticles according paragraph 29, wherein W₁ and W₂ are selected from O or NH; and
X₁ and X₂ are each independently:
a linker of the formula:

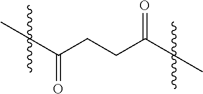 [SMILES: *C(=O)CCC(*)=O]

or a fluorophore of the formula:

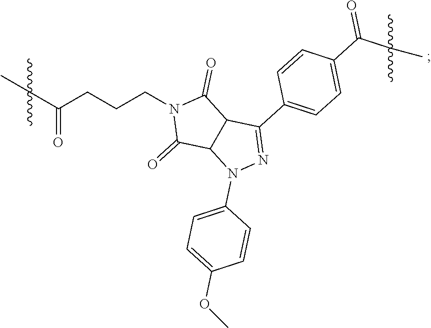 [SMILES: *C(=O)CCCN1C(=O)C2C(c3ccc(C(*)=O)cc3)=NN(c3ccc(OC)cc3)C2C1=O]

and C₁ and C₂ are each a group of the formula:

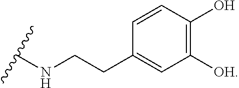 [SMILES: *NCCc1ccc(O)c(O)c1]

31. A process for preparing polydopamine co-polymer nanoparticles according to any one of paragraphs 27 to 30, wherein:
(i) a1 is an integer between 75 and 125;
   a2 is an integer between 75 and 125; and
   b is an integer between 25 and 75; or
(ii) a1 is an integer between 90 and 110;
   a2 is an integer between 90 and 110; and
   b is an integer between 45 and 65.
      32. A process for preparing polydopamine co-polymer nanoparticles according to any one of paragraphs 27 to 31, wherein a1 and a2 are the same.
      33. A process for preparing polydopamine co-polymer nanoparticles according to any one of paragraphs 27 to 32, wherein
a1 is 101;
a2 is 101; and
b is 56 (i.e. Pluronic F127).
   34. A process for preparing polydopamine co-polymer nanoparticles according to any one of paragraphs 24 to 33, wherein the polymerisation of the monomers is conducted in a solvent comprising a mixture of ethanol and water.
   35. A process for preparing polydopamine co-polymer nanoparticles according to any one of paragraphs 24 to 34, wherein, the solvent is selected from:
(i) water comprising 0 to 60% v/v ethanol;
(ii) water comprising 5 to 45% v/v ethanol;
(iii) water comprising 5 to 40% v/v ethanol;
(iv) water comprising 10 to 35% v/v ethanol; or
(v) water comprising 10 to 20% v/v ethanol.
   36. A process for preparing polydopamine co-polymer nanoparticles according to any one of paragraphs 24 to 35, wherein the molar ratio of catecholamine (e.g. dopamine) or DOPAC monomer to monomers of catecholamine (e.g. dopamine) or DOPAC that are covalently bound to a co-polymer of poly(ethylene oxide) and poly(propylene oxide) is selected from:
(i) 5:1 to 150:1;
(ii) 5:1 to 100:1;
(iii) 5:1 to 80:1;
(iv) 5:1 to 75:1;
(v) 5:1 to 50:1;
(vi) 10:1 to 40:1;
(vii) 10:1 to 30:1;
(viii) 15:1 to 25:1; or
(ix) 20:1.
   37. A process for preparing polydopamine co-polymer nanoparticles according to any one of paragraphs 24 to 36, wherein the polymerisation reaction is conducted in the presence of a base (e.g. trizma base).
   38. A process for preparing polydopamine co-polymer nanoparticles according to any one of paragraphs 24 to 37, wherein the polymerisation reaction is conducted at a temperature of between 5 to 35° C., optionally between 15 and 25° C. or 20 and 25° C.
   39. A process for preparing polydopamine co-polymer nanoparticles according to any one of paragraphs 24 to 38, wherein the process further comprises a step of collecting the polydopamine co-polymer nanoparticles formed by the process, optionally by centrifugation, filtration and/or dialysis.
   40. A process for preparing polydopamine co-polymer nanoparticles according to paragraph 39, wherein the process further comprises a step of washing the collected nanoparticles, optionally by resuspending the collected nanoparticles in a suitable vehicle and recollecting the particles by centrifugation.
   41. A process for preparing polydopamine co-polymer nanoparticles according to any one of paragraphs 24 to 40, wherein the process further comprises:
a) adding an additional monomer of dopamine covalently attached to functional moiety to the mixture of the catecholamine (e.g. dopamine) or DOPAC monomer and the monomer of a catecholamine (e.g. dopamine) or DOPAC that is covalently bound to a co-polymer of poly(ethylene oxide) and poly(propylene oxide) and polymerising the monomers to form polydopamine co-polymer nanoparticles having a co-polymer of poly(ethylene oxide) and poly(propylene oxide) and a functional moiety covalently bound thereto; or
b) covalently attaching a functional moiety to the polydopamine co-polymer nanoparticles formed by the process defined in any one paragraphs 20 to 35; and/or
c) adsorbing a functional moiety to the polydopamine co-polymer nanoparticles formed by the process defined in any one paragraphs 20 to 35.
   42. A process for preparing polydopamine co-polymer nanoparticles according paragraph 41, wherein the functional moiety covalently attached or adsorbed to the nanoparticle is a moiety selected from the group consisting of a pharmacologically active agent (e.g. a drug or biologic), a targeting ligand (e.g. a receptor ligand, antibody or nanobody), or an imaging agent (e.g. a detectable moiety, such as a fluorophore, magnetic particles and/or radionuclides).
   43. Polydopamine co-polymer nanoparticles obtainable by, obtained by or directly obtained by a process according to any one of paragraphs 24 to 42.
   44. Polydopamine co-polymer nanoparticles according to any one of paragraphs 1 to 21, or 43, or a pharmaceutical composition according to paragraph 22 or paragraph 23, wherein the polydopamine co-polymer nanoparticles are loaded with a pharmacologically active agent.
   45. Polydopamine co-polymer nanoparticles according to paragraph 44, for use in therapy.
   46. Polydopamine co-polymer nanoparticles according to paragraph 44, wherein the pharmacologically active agent is an anticancer agent and the nanoparticles are for use in the treatment of a cancer.
   47. Polydopamine co-polymer nanoparticles according to paragraph 44, wherein the pharmacologically active agent is an anticancer agent and the polydopamine co-polymer nanoparticles are for use in the treatment of a solid tumour cancers, optionally selected from pancreatic cancer, mesothelioma, bladder cancer, breast cancer, cervical cancer, colon & rectal cancer, endometrial cancer, kidney cancer, lip & oral cancer, liver cancer, melanoma, non-small cell lung cancer, nonmelanoma skin cancer, oral cancer, ovarian cancer, prostate cancer, sarcoma, small cell lung cancer, and thyroid cancer.
   48. Polydopamine co-polymer nanoparticles according to any one of paragraphs 1 to 21, or 43, or a pharmaceutical composition according to paragraph 22 or paragraph 23, wherein the polydopamine co-polymer nanoparticles are loaded with an imaging agent, optionally a fluorophore, magnetic particles, a radionuclide or a photoacoustic imaging agent (e.g. cyanine dyes such indocyanine green (ICG)).
   49. Polydopamine co-polymer nanoparticles or a pharmaceutical composition according to paragraph 43, wherein the polydopamine co-polymer nanoparticles are loaded with a fluorophore.
   50. Polydopamine co-polymer nanoparticles according to any one of paragraphs 1 to 21, 47, 48 or 49, or a pharmaceutical composition according to paragraph 22 or paragraph 23, for use as:
(i) a photoacoustic imaging agent;
(ii) a photothermal therapeutic agent;
(iii) a magnetic resonance imaging (MRI) agent (i,e, an MRI contrast agent); or
(iv) a positron emission tomography (PET) imaging agent.

1. Polydopamine co-polymer nanoparticles comprising polydopamine having a co-polymer of poly(ethylene oxide) and poly(propylene oxide) covalently bound thereto.

2. Polydopamine co-polymer nanoparticles according to claim 1, wherein the polydopamine co-polymer nanoparticles have a particle size of less than or equal to 140 nm; optionally wherein the polydopamine co-polymer nanoparticles have a particle size of 30 to 140 nm.

3. Polydopamine co-polymer nanoparticles according to claim 1 or claim 2, wherein the co-polymer of poly(ethylene oxide) and poly(propylene oxide) is a block co-polymer; optionally wherein the co-polymer of poly(ethylene oxide) and poly(propylene oxide) is a tri-block co-polymer having a central block of poly(propylene oxide) flanked on each side by blocks of poly(ethylene oxide).

4. Polydopamine co-polymer nanoparticles according to any one of the preceding claims, wherein the co-polymer has the formula:
-[poly(ethylene oxide)]-[poly(propylene oxide)]-[poly(ethylene oxide)]-
or
—X₁-[poly(ethylene oxide)]-[poly(propylene oxide)]-[poly(ethylene oxide)]-X₂—
wherein:
X₁ and X₂ are each independently:
a linker group that connects the co-polymer to the polydopamine; or
a detectable moiety (e.g. a fluorophore) that connects the co-polymer to the polydopamine.

5. Polydopamine co-polymer nanoparticles according to any one of the preceding claims, wherein the co-polymer has the formula: 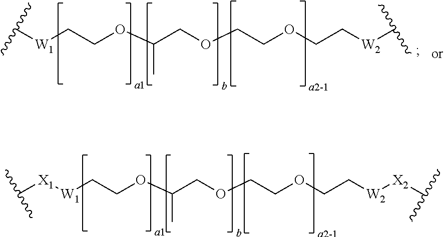
wherein:
W₁ and W₂ are selected from O or NH; or
W₁ and W₂ are selected from: 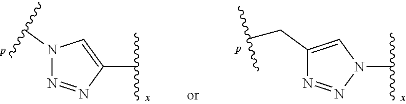 [SMILES: *c1cn(*)nn1 ; *Cc1cn(*)nn1]
wherein
p indicates the bond to the co-polymer;
x indicates the bond to the X₁ or X₂ group, if present, or the polydopamine;
a1 is an integer between 50 and 150;
a2 is an integer between 50 and 150;
b is an integer between 20 and 80; and
X₁ and X₂ are as defined in claim 4.

6. Polydopamine co-polymer nanoparticles according to claim 5, wherein:
W₁ and W₂ are as defined in claim 9;
a1 is an integer between 75 and 125;
a2 is an integer between 75 and 125; and
b is an integer between 25 and 75;
optionally wherein:
a1 is an integer between 90 and 110;
a2 is an integer between 90 and 110; and
b is an integer between 45 and 65; and/or
a1 and a2 are the same;
further optionally wherein:
a1 is 101;
a2 is 101; and
b is 56 (i.e. Pluronic F127).

7. Polydopamine co-polymer nanoparticles according to claim 5 or 6 wherein:
W₁ and W₂ are selected from O or NH; and
X₁ and X₂ are each independently:
(i) a linker of the formula:
—C(═O)—[CH₂]ₙ—C(═O)—
wherein:
n is an integer from 1 to 10; or
(ii) a fluorophore.

8. Polydopamine co-polymer nanoparticles according to any one of claims 5 to 7, wherein either:
A) W₁ and W₂ are selected from O or NH; and
X₁ and X₂ are each independently:
(i) a linker of the formula:
—C(═O)—[CH₂]ₙ—C(═O)—
wherein n is an integer from 1 to 10; or
(ii) a fluorophore of the formula: 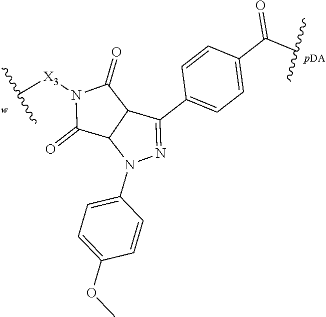
wherein:
w indicates the bond to the W₁ or W₂ group;
pDA indicates the bond to the polydopamine;
X₃ is selected from:
(i) —C(═O)—[CH₂]ₙ₁—. wherein n1 is an integer from 2 to 10;
(ii) —[CH₂]ₙ₂—, wherein n2 is an integer from 2 to 10;
(iii) —C(═O)—CH₂CH₂—[OCH₂CH₂]ₙ₃—, n3 is an integer from 2 to 10;
(iii) a fluorophore of the formula: 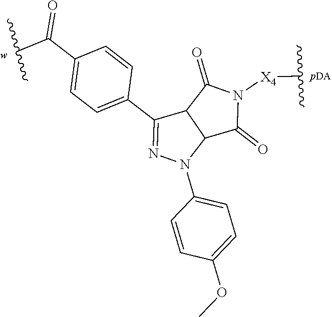
wherein:
w indicates the bond to the W₁ or W₂ group;
pDA indicates the bond to the polydopamine;
X₄ is selected from:
(i) —[CH₂]ₙ₄—C(═O)—, wherein n4 is an integer from 2 to 10;
(ii) —[CH₂]ₙ₅—, wherein n5 is an integer from 2 to 10;
(iii) —[CH₂CH₂O]ₙ₆—CH₂CH₂—C(═O)—, wherein n6 is an integer from 2 to 10; or
B) W₁ and W₂ are each a group of the formula: 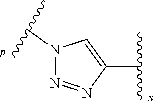 [SMILES: *c1cn(*)nn1]
wherein
ρ indicates the bond to the co-polymer;
x indicates the bond to the X₁ or X₂ group;
X₁ and X₂ are each selected from:
(i) —CH₂—
(ii) —CH₂CH₂—[OCH₂CH₂]ₙ₇—, wherein n7 is an integer from 2 to 10;
(iii) —CH₂—C(═O)—
(iv) —CH₂CH₂—[OCH₂CH₂]ₙ₈—NH—C(═O)—, wherein n8 is an integer from 2 to 10;
(v) —CH₂CH₂—[OCH₂CH₂]ₙ₉—O—C(═O)—, wherein n9 is an integer from 2 to 10; and
e) W₁ and W₂ are each a group of the formula: 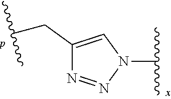 [SMILES: *Cc1cn(*)nn1]
wherein
p indicates the bond to the co-polymer;
x indicates the bond to the X₁ or X₂ group;
X₁ and X₂ are each selected from:
(i) —CH₂CH₂—[OCH₂CH₂]ₙ₁₀, wherein n10 is an integer from 2 to 10;
(ii) —CH₂CH₂—[OCH₂CH₂]ₙ₁₁, wherein n11 is an integer from 2 to 10;
(iii) —[CH₂CH₂O]ₙ₁₂—C(═O)—CH₂CH₂—C(═O), wherein n12 is an integer from 2 to 10; and
(vi) —[CH₂CH₂O]ₙ₁₃—CH₂CH₂—NH—C(═O)—, wherein n13 is an integer from 2 to 10;
(vii) —CH₂CH₂—[OCH₂CH₂]ₙ₁₄—O—C(═O), wherein n14 is an integer from 2 to 10;
(viii) —[CH₂CH₂O]ₙ₁₅—C(═O)—CH₂CH₂—C(═O), wherein n15 is an integer from 2 to 10.

9. Polydopamine co-polymer nanoparticles according to any one of claims 5 to 8, wherein W₁ and W₂ are selected from O or NH; and
X₁ and X₂ are each independently:
(i) a linker of the formula:
—C(═O)—[C H₂]ₙ—C(═O)—
wherein n is an integer from 2 to 8; or
(ii) a fluorophore of the formula: 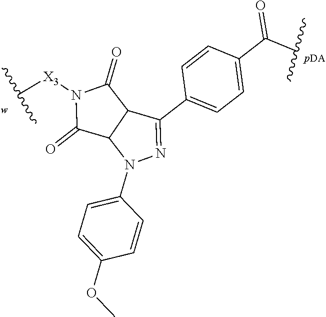
wherein:
w indicates the bond to the W₁ or W₂ group;
pDA indicates the bond to the polydopamine;
X₃ is selected from:
(i) —C(═O)—[CH₂]ₙ₁—. wherein n1 is an integer from 2 to 8;
(ii) —[CH₂]ₙ₂—, wherein n2 is an integer from 2 to 8;
(iii) —C(═O)—CH₂CH₂—[OCH₂CH₂]ₙ₃—, n3 is an integer from 2 to 8;
(iii) a fluorophore of the formula: 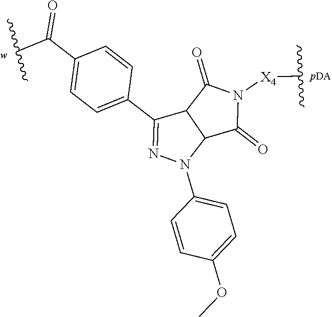
wherein:
w indicates the bond to the W₁ or W₂ group;
pDA indicates the bond to the polydopamine;
X₄ is selected from:
(i) —[CH₂]ₙ₄—C(═O). wherein n4 is an integer from 2 to 8;
(ii) —[CH₂]ₙ₅—, wherein n5 is an integer from 2 to 8;
(iii) —CH₂CH₂—[CH₂CH₂O]ₙ₆—C(═O)—, wherein n6 is an integer from 2 to 8;
optionally wherein X₁ and X₂ are each independently:
(i) a linker of the formula:
—C(═O)—[C H₂]ₙ—C(═O)—
wherein n is an integer from 2 to 8; or
(ii) a fluorophore of the formula: 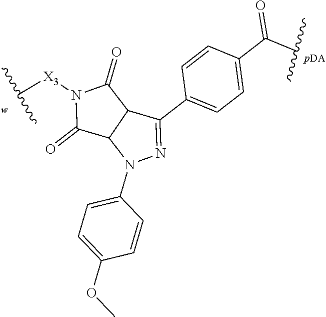
wherein:
w indicates the bond to the W₁ or W₂ group;
pDA indicates the bond to the polydopamine;
X₃ is —C(═O)—[CH₂]ₙ₁—. wherein n1 is an integer from 2 to 8;
further optionally wherein W₁ and W₂ are selected from O or NH, and X₁ and X₂ are each independently a linker of the formula: 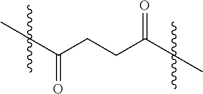 [SMILES: *C(=O)CCC(*)=O]
or a fluorophore of the formula:

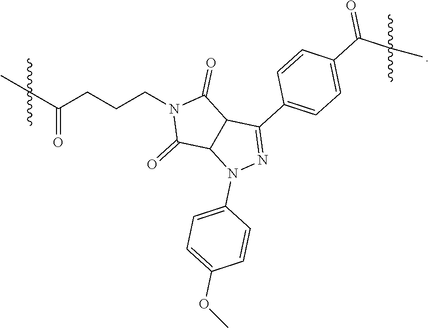 [SMILES: *C(=O)CCCN1C(=O)C2C(c3ccc(C(*)=O)cc3)=NN(c3ccc(OC)cc3)C2C1=O]

10. Polydopamine co-polymer nanoparticles according to any one of claims 5 to 9, wherein W₁ and W₂ are each O.

11. Polydopamine co-polymer nanoparticles according to any one of the preceding claims, wherein the polydopamine co-polymer nanoparticles further comprise one or more functional moieties covalently attached or adsorbed to the nanoparticle; optionally wherein the one or more functional moieties covalently attached or adsorbed to the nanoparticle is a moiety selected from the group consisting of a pharmacologically active agent (e.g. a drug or biologic), a targeting ligand (e.g. a receptor ligand, antibody or nanobody), or an imaging agent (e.g. a detectable moiety, such as a fluorophore, magnetic particles and/or radionuclides).

12. A pharmaceutical composition comprising polydopamine co-polymer nanoparticles according to any one of claims 1 to 11, a pharmacologically active agent (e.g. a drug or biologic)) or an imaging agent (e.g. a detectable moiety, such as a fluorophore, magnetic particles and/or radionuclides, and a pharmaceutically acceptable excipient;
optionally wherein the pharmacologically active agent (e.g. a drug or biologic) or imaging agent (e.g. a detectable moiety, such as a fluorophore, magnetic particles and/or radionuclides) is dispersed in an aqueous vehicle.

13. A process for preparing polydopamine co-polymer nanoparticles according to any one of claims 1 to 11, the process comprising polymerising a catecholamine (e.g. dopamine) or DOPAC monomer with a monomer of a catecholamine (e.g. dopamine) or DOPAC, that is covalently bound to a co-polymer of poly(ethylene oxide) and poly(propylene oxide) to form polydopamine co-polymer nanoparticles having a co-polymer of poly(ethylene oxide) and poly(propylene oxide) covalently bound thereto;
optionally wherein, in the monomer of a catecholamine (e.g. dopamine) or DOPAC, that is covalently bound to a co-polymer of poly(ethylene oxide) and poly(propylene oxide), the co-polymer of poly(ethylene oxide) and poly(propylene oxide) is a tri-block co-polymer having a central block of poly(propylene oxide) flanked on each side by blocks of poly(ethylene oxide);
further optionally wherein, in the monomer of a catecholamine (e.g. dopamine) or DOPAC that is covalently bound to a co-polymer of poly(ethylene oxide) and poly(propylene oxide), the co-polymer of poly(ethylene oxide) and poly(propylene oxide) has the formula:
-[poly(ethylene oxide)]-[poly(propylene oxide)]-[poly(ethylene oxide)]-
or
—X₁-[poly(ethylene oxide)]-[poly(propylene oxide)]-[poly(ethylene oxide)]-X₂—
wherein:
X₁ and X₂ are each independently:
a linker group that connects the co-polymer to the polydopamine; or
a detectable moiety (e.g. a fluorophore) that connects the co-polymer to the polydopamine.

14. A process for preparing polydopamine co-polymer nanoparticles according to claim 13, wherein the monomer of a catecholamine (e.g. dopamine) or DOPAC that is covalently bound to a co-polymer of poly(ethylene oxide) and poly(propylene oxide) has the formula: 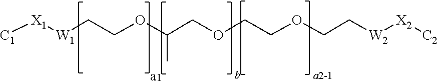
wherein:
W₁ and W₂ are selected from O or NH; or
W₁ and W₂ are selected from: 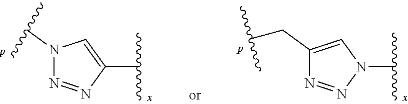 [SMILES: *c1cn(*)nn1 ; *Cc1cn(*)nn1]
wherein:
p indicates the bond to the co-polymer;
x indicates the bond to the X₁ or X₂ group;
a1 is an integer between 50 and 150;
a2 is an integer between 50 and 150;
b is an integer between 20 and 80; and
X₁ and X₂ are each independently:
a linker group; or
a detectable moiety (e.g. a fluorophore); and
C₁ and C₂ are a catecholamine or DOPAC.
optionally wherein:
A) W₁ and W₂ are selected from O or NH; and
either
i) X₁ and X₂ are each a group of the formula:
—C(═O)—[C H₂]ₙ—C(═O)—
wherein n is an integer from 1 to 10; or
ii) X₁ and X₂ are each a group of the formula: 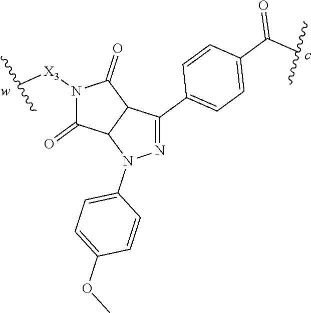
wherein:
w indicates the bond to the W₁ or W₂ group;
c indicates the bond to the C₁ or C₂ group;
X₃ is selected from:
(i) —C(═O)—[CH₂]ₙ₁—. wherein n1 is an integer from 2 to 10;
(ii) —[CH₂]ₙ₂—, wherein n2 is an integer from 2 to 10;
(iii) —C(═O)—CH₂CH₂—[OCH₂CH₂]ₙ₃—, n3 is an integer from 2 to 10; and
C₁ and C₂ are a group of a formula selected from: 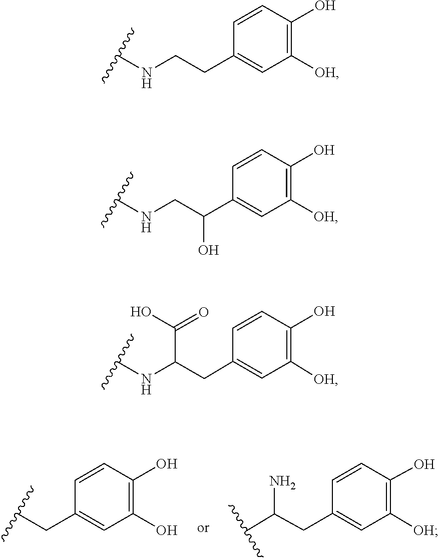 [SMILES: *NCCc1ccc(O)c(O)c1 ; *NCC(O)c1ccc(O)c(O)c1 ; *NC(Cc1ccc(O)c(O)c1)C(=O)O ; *Cc1ccc(O)c(O)c1 ; *C(N)Cc1ccc(O)c(O)c1]
B) W₁ and W₂ are selected from O or NH; and
X₁ and X₂ are each a group of the formula: 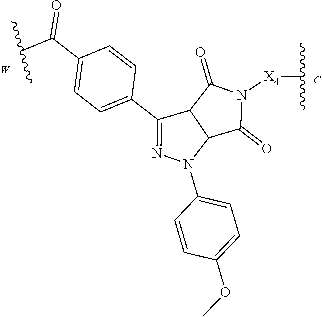
w indicates the bond to the W₁ or W₂ group:
c indicates the bond to the C₁ or C₂ group;
either
i) X₄ is selected from:
(i) —[CH₂]ₙ₄—C(═O). wherein n4 is an integer from 2 to 10;
(ii) —[CH₂]ₙ₅—, wherein n5 is an integer from 2 to 10;
(iii) —[CH₂CH₂O]ₙ₆—CH₂CH₂—C(═O)—, wherein n6 is an integer from 2 to 10; and
C₁ and C₂ are each a group selected from: 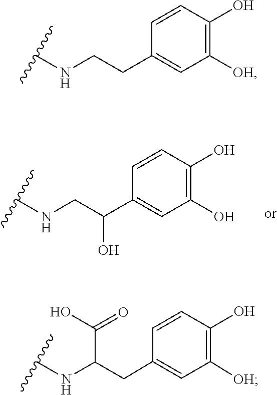 [SMILES: *NCCc1ccc(O)c(O)c1 ; *NCC(O)c1ccc(O)c(O)c1 ; *NC(Cc1ccc(O)c(O)c1)C(=O)O]
or
ii) X₄ is selected from:
(i) —[CH₂]ₙ₄—C(═O). wherein n4 is an integer from 2 to 10;
(ii) —[CH₂]ₙ₅—C(═O), wherein n5 is an integer from 2 to 10;
(iii) —[CH₂CH₂O]ₙ₆—CH₂CH₂—C(═O)—, wherein n6 is an integer from 2 to 10; and
C₁ and C₂ are each a group of the formula: 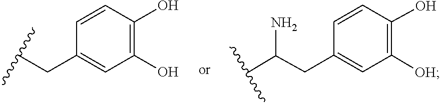 [SMILES: *Cc1ccc(O)c(O)c1 ; *C(N)Cc1ccc(O)c(O)c1]
C) W₁ and W₂ are each a group of the formula: 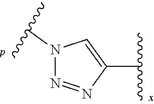 [SMILES: *c1cn(*)nn1]
wherein
p indicates the bond to the co-polymer;
x indicates the bond to the X₁ or X₂ group;
wherein either
i) X₁ and X₂ are each selected from:
(i) —CH₂—
(ii) —CH₂CH₂—[OCH₂CH₂]ₙ₇—, wherein n7 is an integer from 2 to 10; and
C₁ and C₂ are a group of a formula selected from: 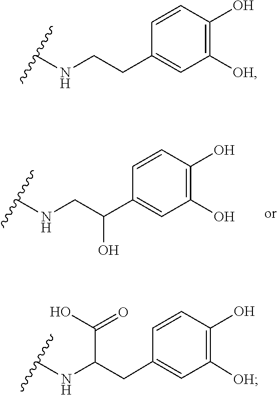 [SMILES: *NCCc1ccc(O)c(O)c1 ; *NCC(O)c1ccc(O)c(O)c1 ; *NC(Cc1ccc(O)c(O)c1)C(=O)O]
or
ii) X₁ and X₂ are each selected from:
(i) —CH₂—C(═O)—;
(ii) —CH₂CH₂—[OCH₂CH₂]ₙ₈NH—C(═O)—, wherein n8 is an integer from 2 to 10;
(iii) —CH₂CH₂—[OCH₂CH₂]ₙ₉—O—C(═O)—, wherein n9 is an integer from 2 to 10; and
C₁ and C₂ are a group of the formula: 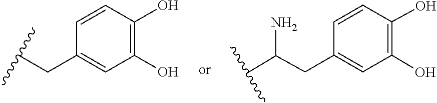 [SMILES: *Cc1ccc(O)c(O)c1 ; *C(N)Cc1ccc(O)c(O)c1]
D) W₁ and W₂ are each a group of the formula: 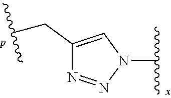 [SMILES: *Cc1cn(*)nn1]
wherein
p indicates the bond to the co-polymer;
x indicates the bond to the X₁ or X₂ group;
wherein either
i) X₁ and X₂ are each selected from:
(i) —CH₂CH₂—[OCH₂CH₂]ₙ10, wherein n10 is an integer from 2 to 10;
(ii) —CH₂CH₂—[OCH₂CH₂]ₙ₁₁ wherein n11 is an integer from 2 to 10;
(iii) —[CH₂CH₂O]ₙ₁₂—C(═O)—CH₂CH₂—C(═O), wherein n12 is an integer from 2 to 10; and
C₁ and C₂ are a group of the formula: 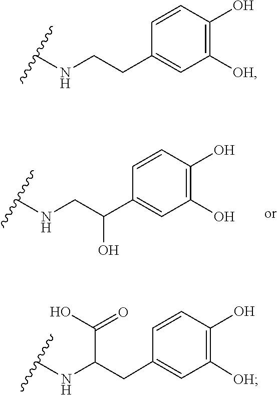 [SMILES: *NCCc1ccc(O)c(O)c1 ; *NCC(O)c1ccc(O)c(O)c1 ; *NC(Cc1ccc(O)c(O)c1)C(=O)O]
ii) X₁ and X₂ are each a group selected from:
(i) —[CH₂CH₂O]ₙ₁₃—CH₂CH₂—NH—C(═O)—, wherein n13 is an integer from 2 to 10;
(ii) —CH₂CH₂—[OCH₂CH₂]ₙ₁₄—O—C(═O), wherein n14 is an integer from 2 to 10;
(iii) —[CH₂CH₂O]ₙ₁₅—C(═O)—CH₂CH₂—C(═O), wherein n15 is an integer from 2 to 10;
and
C₁ and C₂ are a group of the formula:

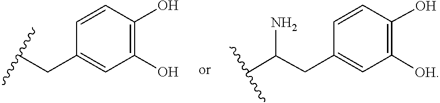 [SMILES: *Cc1ccc(O)c(O)c1 ; *C(N)Cc1ccc(O)c(O)c1]

15. A process for preparing polydopamine co-polymer nanoparticles according to claim 14, wherein W₁ and W₂ are selected from O or NH; and
either
i) X₁ and X₂ are each a group of the formula:
—C(═O)—[C H₂]ₙ—C(═O)—
wherein n is an integer from 1 to 10; or
ii) X₁ and X₂ are each a group of the formula: 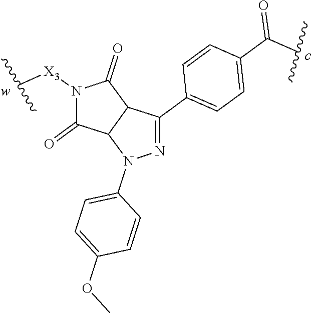
wherein:
w indicates the bond to the W₁ or W₂ group;
c indicates the bond to the C₁ or C₂ group; and
X₃ is selected from:
(i) —C(═O)—[CH₂]ₙ₁—. wherein n1 is an integer from 2 to 10;
(ii) —[CH₂]ₙ₂—, wherein n2 is an integer from 2 to 10; and
(iii) —C(═O)—CH₂CH₂—[OCH₂CH₂]ₙ₃—, n3 is an integer from 2 to 10;
and C₁ and C₂ are each a group: 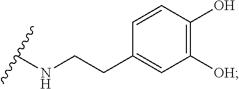 [SMILES: *NCCc1ccc(O)c(O)c1]
optionally wherein X₁ and X₂ are each independently:
a linker of the formula: 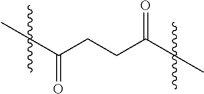 [SMILES: *C(=O)CCC(*)=O]
or a fluorophore of the formula: 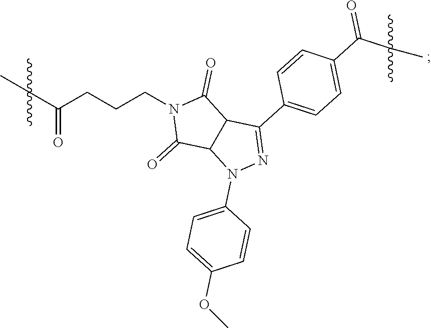 [SMILES: *C(=O)CCCN1C(=O)C2C(c3ccc(C(*)=O)cc3)=NN(c3ccc(OC)cc3)C2C1=O]
and C₁ and C₂ are each a group of the formula:

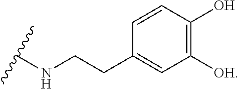 [SMILES: *NCCc1ccc(O)c(O)c1]

16. A process for preparing polydopamine co-polymer nanoparticles according to any one of claims 13 to 15, wherein:
a1 is an integer between 75 and 125;
a2 is an integer between 75 and 125; and
b is an integer between 25 and 75;
optionally wherein:
a1 is an integer between 90 and 110;
a2 is an integer between 90 and 110; and
b is an integer between 45 and 65;
and/or
a1 and a2 are the same;
further optionally wherein
a1 is 101;
a2 is 101; and
b is 56 (i.e. Pluronic F127).

17. A process for preparing polydopamine co-polymer nanoparticles according to any one of claims 13 to 16, wherein the polymerisation of the monomers is conducted in a solvent comprising a mixture of ethanol and water;
optionally wherein the solvent is selected from:
(i) water comprising 0 to 60% v/v ethanol;
(ii) water comprising 5 to 45% v/v ethanol;
(iii) water comprising 5 to 40% v/v ethanol;
(iv) water comprising 10 to 35% v/v ethanol; or
(v) water comprising 10 to 20% v/v ethanol.

18. A process for preparing polydopamine co-polymer nanoparticles according to any one of claims 13 to 17, wherein the molar ratio of catecholamine (e.g. dopamine) or DOPAC monomer to monomers of catecholamine (e.g. dopamine) or DOPAC that are covalently bound to a co-polymer of poly(ethylene oxide) and poly(propylene oxide) is selected from:
(i) 5:1 to 150:1;
(ii) 5:1 to 100:1;
(iii) 5:1 to 80:1:
(iv) 5:1 to 75:1;
(v) 5:1 to 50:1;
(vi) 10:1 to 40:1;
(vii) 10:1 to 30:1;
(viii) 15:1 to 25:1; or
(ix) 20:1.

19. A process for preparing polydopamine co-polymer nanoparticles according to any one of claims 13 to 18, wherein the polymerisation reaction is conducted in the presence of a base (e.g. trizma base); and/or
the polymerisation reaction is conducted at a temperature of between 5 to 35° C., optionally between 15 and 25° C. or 20 and 25° C.; and/or
the process further comprises a step of collecting the polydopamine co-polymer nanoparticles formed by the process, optionally by centrifugation, filtration and/or dialysis; optionally wherein the process further comprises a step of washing the collected nanoparticles, optionally by resuspending the collected nanoparticles in a suitable vehicle and recollecting the particles by centrifugation.

20. A process for preparing polydopamine co-polymer nanoparticles according to any one of claims 13 to 19, wherein the process further comprises:
d) adding an additional monomer of dopamine covalently attached to functional moiety to the mixture of the catecholamine (e.g. dopamine) or DOPAC monomer and the monomer of a catecholamine (e.g. dopamine) or DOPAC that is covalently bound to a co-polymer of poly(ethylene oxide) and poly(propylene oxide) and polymerising the monomers to form polydopamine co-polymer nanoparticles having a co-polymer of poly(ethylene oxide) and poly(propylene oxide) and a functional moiety covalently bound thereto; or
e) covalently attaching a functional moiety to the polydopamine co-polymer nanoparticles formed by the process defined in any one claims 13 to 19; and/or
f) adsorbing a functional moiety to the polydopamine co-polymer nanoparticles formed by the process defined in any one claims 13 to 19;
optionally wherein the functional moiety covalently attached or adsorbed to the nanoparticle is a moiety selected from the group consisting of a pharmacologically active agent (e.g. a drug or biologic), a targeting ligand (e.g. a receptor ligand, antibody or nanobody), or an imaging agent (e.g. a detectable moiety, such as a fluorophore, magnetic particles and/or radionuclides).

21. Polydopamine co-polymer nanoparticles obtainable by, obtained by or directly obtained by a process according to any one of claims 13 to 20.

22. Polydopamine co-polymer nanoparticles according to any one of claims 1 to 11, or 21, or a pharmaceutical composition according to claim 12, wherein the polydopamine co-polymer nanoparticles are loaded with one or more of:
a) a pharmacologically active agent; optionally an anticancer agent or a nucleic acid; or
b) an imaging agent; optionally a fluorophore, magnetic particles, a radionuclide or a photoacoustic imaging agent (e.g. cyanine dyes such indocyanine green (ICG)).

23. Polydopamine co-polymer nanoparticles according to claim 22, for use in therapy.

24. Polydopamine co-polymer nanoparticles according to claim 22, wherein the polydopamine co-polymer nanoparticles are loaded with an anticancer agent and the nanoparticles are for use in the treatment of a cancer; optionally solid tumour cancers, optionally selected from pancreatic cancer, mesothelioma, bladder cancer, breast cancer, cervical cancer, colon & rectal cancer, endometrial cancer, kidney cancer, lip & oral cancer, liver cancer, melanoma, non-small cell lung cancer, nonmelanoma skin cancer, oral cancer, ovarian cancer, prostate cancer, sarcoma, small cell lung cancer, and thyroid cancer.

25. Polydopamine co-polymer nanoparticles according to claim 22, wherein the polydopamine co-polymer nanoparticles are loaded with an imaging agent, (e.g. a fluorophore, magnetic particles, a radionuclide or a photoacoustic imaging agent (e.g. cyanine dyes such indocyanine green) and are for use as:
(i) a photoacoustic imaging agent;
(ii) a photothermal therapeutic agent;
(iii) a magnetic resonance imaging (MRI) agent (i,e, an MRI contrast agent); or
(iv) a positron emission tomography (PET) imaging agent.

26. Polydopamine co-polymer nanoparticles according to any one of claims 1 to 11, or 21, or a pharmaceutical composition according to claim 12, wherein the polydopamine co-polymer nanoparticles are loaded with a nucleic acid;
optionally wherein the nucleic acid is selected from DNA or RNA;
further optionally wherein the polydopamine co-polymer nanoparticles are further functionalised with:
a poly(amino acid) which is positively charged at pH 7; or
ferulic acid.

27. Polydopamine co-polymer nanoparticles according to claim 26, for use in the delivery of a nucleic acid in therapy.

28. The use of polydopamine co-polymer nanoparticles according to claim 26, in:
a) the transfection of cells in vitro or in vivo;
b) the transfection of plant cells; or
c) the transfection of human or animal cells.

29. Polydopamine co-polymer nanoparticles according to any one of claims 1 to 11, or 21, or a pharmaceutical composition according to claim 12, wherein the polydopamine co-polymer nanoparticles are loaded with one or more of:
a) a drug-peptide conjugate, such as an ATRA conjugated peptide;
b) a tumour penetrating peptide such as an RGD peptide; and
c) a dye, such as Rhodamine-TEG-NH₂ or Fluorescein;
and an additional pharmacologically active agent such as a drug.


SEQUENCE LISTING (17 sequences)

SEQ ID NO: 1 - protein, 9 aa, synthetic construct
  REGION 1..9: note = Peptide
GPQGIFGQK

SEQ ID NO: 2 - protein, 8 aa, synthetic construct
  REGION 1..8: note = Peptide
GPVGLIGK

SEQ ID NO: 3 - protein, 8 aa, synthetic construct
  REGION 1..8: note = Peptide
GPLGIAGQ

SEQ ID NO: 4 - protein, 6 aa, synthetic construct
  REGION 1..6: note = Peptide
PLGLAG

SEQ ID NO: 5 - protein, 8 aa, synthetic construct
  REGION 1..8: note = Peptide
CPLGLAGG

SEQ ID NO: 6 - protein, 9 aa, synthetic construct
  REGION 1..9: note = Peptide
GPLGVRGDG

SEQ ID NO: 7 - protein, 10 aa, synthetic construct
  REGION 1..10: note = Peptide
VPLSLYSGCG

SEQ ID NO: 8 - protein, 20 aa, synthetic construct
  REGION 1..20: note = Peptide
GERGPPGPQGARGFZGTPGL

SEQ ID NO: 9 - protein, 12 aa, synthetic construct
  REGION 1..12: note = Peptide
CGPQGIWGQGCR

SEQ ID NO: 10 - protein, 6 aa, synthetic construct
  REGION 1..6: note = Peptide
PVGLIG

SEQ ID NO: 11 - protein, 7 aa, synthetic construct
  REGION 1..7: note = Peptide
RSWMGLP

SEQ ID NO: 12 - protein, 9 aa, synthetic construct
  REGION 1..9: note = Peptide
GPLGIAGQC

SEQ ID NO: 13 - protein, 6 aa, synthetic construct
  REGION 1..6: note = Peptide
PLGLAG

SEQ ID NO: 14 - protein, 16 aa, synthetic construct
  REGION 1..16: note = Peptide
AGFSGPLGMWSAGSFG

SEQ ID NO: 15 - protein, 8 aa, synthetic construct
  REGION 1..8: note = TAMRA NLS SV40
  SITE 1: note = N-terminal peptide bonded TAMRA
PKKKRKVC

SEQ ID NO: 16 - protein, 9 aa, synthetic construct
  REGION 1..9: note = MMP cleavable peptide sequence
QGAIGLPGC

SEQ ID NO: 17 - protein, 9 aa, synthetic construct
  REGION 1..9: note = ATRA-MMP cleavable peptide linker
  SITE 1: note = N-terminal peptide bonded ATRA
QGAIGLPGC